SEQUENCE LISTING (28 sequences)

SEQ ID NO: 1 - DNA, 10 bp, Artificial Sequence
  Cal_1
aaaaaaaaaa

SEQ ID NO: 2 - DNA, 10 bp, Artificial Sequence
  Cal_2
gggggggggg

SEQ ID NO: 3 - DNA, 10 bp, Artificial Sequence
  Cal_3
cccccccccc

SEQ ID NO: 4 - DNA, 10 bp, Artificial Sequence
  Cal_4
tttttttttt

SEQ ID NO: 5 - DNA, 10 bp, Artificial Sequence
  Cal_5
aaagaaaaca

SEQ ID NO: 6 - DNA, 10 bp, Artificial Sequence
  Cal_6
gggtgggagg

SEQ ID NO: 7 - DNA, 10 bp, Artificial Sequence
  Cal_7
cctcccaccc

SEQ ID NO: 8 - DNA, 10 bp, Artificial Sequence
  Cal_8
tgttttcttt

SEQ ID NO: 9 - DNA, 10 bp, Artificial Sequence
  Cal_9
agaatagaat

SEQ ID NO: 10 - DNA, 10 bp, Artificial Sequence
  Cal_10
cggaggagcg

SEQ ID NO: 11 - DNA, 10 bp, Artificial Sequence
  Cal_11
cgctccgcct

SEQ ID NO: 12 - DNA, 10 bp, Artificial Sequence
  Cal_12
cttctattat

SEQ ID NO: 13 - DNA, 10 bp, Artificial Sequence
  Cal_13
aacgcatcca

SEQ ID NO: 14 - DNA, 10 bp, Artificial Sequence
  Cal_14
gtgcgattgt

SEQ ID NO: 15 - DNA, 10 bp, Artificial Sequence
  Gen_1
cccactttct

SEQ ID NO: 16 - DNA, 10 bp, Artificial Sequence
  Gen_2
acgaggttct

SEQ ID NO: 17 - DNA, 10 bp, Artificial Sequence
  Gen_3
gcgcagggag

SEQ ID NO: 18 - DNA, 10 bp, Artificial Sequence
  Gen_4
gatcagcgcg

SEQ ID NO: 19 - DNA, 10 bp, Artificial Sequence
  Gen_5
cccctcctct

SEQ ID NO: 20 - DNA, 10 bp, Artificial Sequence
  Gen_6
ggtggcgaac

SEQ ID NO: 21 - DNA, 10 bp, Artificial Sequence
  Gen_7
aagcgcaacg

SEQ ID NO: 22 - DNA, 10 bp, Artificial Sequence
  Gen_8
cttcgtcctc

SEQ ID NO: 23 - DNA, 10 bp, Artificial Sequence
  Gen_9
agcggctcta

SEQ ID NO: 24 - DNA, 10 bp, Artificial Sequence
  Gen_10
ggtgggtggg

SEQ ID NO: 25 - DNA, 10 bp, Artificial Sequence
  Gen_11
gaccgggagc

SEQ ID NO: 26 - DNA, 10 bp, Artificial Sequence
  Gen_12
gccaggttgt

SEQ ID NO: 27 - DNA, 10 bp, Artificial Sequence
  Gen_13
gccaatgtct

SEQ ID NO: 28 - DNA, 10 bp, Artificial Sequence
  Gen_14
aagccccagc

(19) United States
(12) Patent Application Publication
(10) Pub. No.: US 2020/0240845 A1
(43) Pub. Date: Jul. 30, 2020

(54) SINGLE-MOLECULE OPTICAL SEQUENCE IDENTIFICATION OF NUCLEIC ACIDS AND AMINO ACIDS FOR COMBINED SINGLE-CELL OMICS AND BLOCK OPTICAL CONTENT SCORING (BOCS): DNA K-MER CONTENT AND SCORING FOR RAPID GENETIC BIOMARKER IDENTIFICATION AT LOW COVERAGE

(71) Applicant: The Regents of the University of Colorado, a body corporate, Denver, CO (US)

(72) Inventors: Prashant Nagpal, Lafayette, CO (US); Gary R. Abel, Jr., Lafayette, CO (US); Lee E. Korshoj, Denver, CO (US); Ameya Gajanan Prabhune, Boulder, CO (US)

(21) Appl. No.: 16/705,112

(22) Filed: Dec. 5, 2019

Related U.S. Application Data
(60) Provisional application No. 62/775,736, filed on Dec. 5, 2018.

(51) Int. Cl. G01J 3/44; G06F 17/18; G16B 15/00; G16B 30/10; G16B 30/20

(52) U.S. Cl. CPC G01J 3/44; G06F 17/18; G16B 30/20; G16B 30/10; G16B 15/00

(57) ABSTRACT

Optical fingerprints for label-free high-throughput (epi)genomics, transcriptomics, and proteomics profiling of single cells. Vibrational spectroscopy signatures combined with a molecular identification algorithm rooted in machine learning enables identification of nucleic acids and amino acids, and their molecular variations, thereby identifying genetic variation by mapping heterogeneity and identifying low copy-number variants. Additional embodiments include the BOCS algorithm which takes measurements of DNA k-mer content from high-throughput single-molecule Raman spectroscopy measurements and maps them to gene databases for probabilistic determination of genetic biomarkers at low coverages. Starting with a log of measured k-mer content blocks (B1 . . . Bn as shown) and a genetic biomarker database (excerpts from the MEGARes antibiotic resistance database are shown), the blocks are individually aligned to each gene in the database based on content. This alignment consists of finding all match locations for the k-mer block content within a gene via translating through the gene one nucleotide at a time and looking at fragments of length k. For each block, a raw probability can be calculated for each gene based on the number of matches for the k-mer block content within the gene, length of the k-mer block, and length of the gene (calculation shown in the schematic). As more blocks are analyzed, probabilities are compounded and genes in the database are ranked. The gene(s) from which the Raman-analyzed k-mer blocks originate quickly generate the top probabilities and can often be determined in coverages <<1.0, meaning that only a small fraction of the gene blocks need to be analyzed for identification of a specific genetic biomarker.

### STATEMENT OF FEDERALLY SPONSORED RESEARCH

This invention was made with support under a grant by the W. M. Keck Foundation, and through the National Science Foundation Soft Materials (MRSEC) at the University of Colorado through NSF Award DMR 1420736, and from the National Science Foundation Graduate Research Fellowship Program under Grant Nos. DGE 1144083 and 1650115. The government has certain rights in the invention.

### TECHNICAL FIELD

The inventive technology includes compositions, devices, processes, methods, and systems are directed to rapid and accurate optical fingerprinting, identification, and sequencing of amino acid and other macromolecules. Additional inventive aspects of the invention include novel systems and methods for bioinformatics algorithms capable of using the high-throughput content k-mers for rapid, broad spectrum identification of genetic biomarkers.

### BACKGROUND OF THE INVENTION

Single-molecule sequencing and mapping of molecular variations in polynucleotides, such as DNA, RNA, and polypeptides can lead to significant improvements in precise diagnosis and treatment of a variety of diseases. First, sequencing of low-copy-number cells without amplification could prove vital for pathogen identification, prenatal care, and diagnosis of circulating tumor cells. Second, an integrated platform capable of single-molecule proteome, genome, transcriptome, and epigenome sequencing could lead to rapid and accurate disease biomarker identification. The lack of such studies at the single-cell level leads to extended controversies and an absence of clear evidence for molecular variations, sometimes at both the genetic and enzymatic levels, as a causative agent for the disease. An example of such impeded progress is the use of epigenetic markers for cancer identification. While several years of research have led to the identification of methylation as an epigenetic marker for cancer cells, it requires a separate and tedious bisulfite sequencing process, which suffers from issues such as incomplete conversion, DNA degradation, and an inability to distinguish between different 5-methylcytosine derivatives. Interconversion between 5-methylcytosine and 5-hydroxymethylcytosine and lack of a direct identification method (current techniques use antibody-based immunofluorescence and immunohistochemistry approaches, immuno-dot blots, and liquid chromatography coupled with mass spectrometry), has prevented its confirmation as a biomarker, and a better understanding of its role in stem cells and tumorigenesis. Further, identification of other new molecular markers and their role in cancer also requires protracted and indirect studies to infer their role. Even for less prevalent or “rare” diseases (affecting less than 200,000 patients each year in the U.S.), in the past 25 years, only about 50% of the 7,000 rare monogenic disease-causing genes have been identified. Together this affects millions without an accurate diagnostic method for identification and therapeutic treatment.

Unfortunately, current sequencing techniques rely on expensive and labor-intensive enzymatic amplification of samples, which introduce amplification bias and provide a statistically significant ensemble-averaged sequence, which often lacks detection of population heterogeneity and information that can be vital for medical intervention. While studies in single-cell genomics have outlined the potential of single-molecule sequencing for medicine and non-invasive clinical applications, these studies involved enzymatic amplification of DNA and subsequent sequencing using traditional sequencing tools. In order to assess the sensitivity required for non-amplified samples, a single prokaryotic cell (˜10⁻¹⁵ liter) with one copy of DNA corresponds to a concentration of (1/(6.023×10²³)/10¹⁵ mol/L) nM, with similar concentration magnitude for low copy number variants, and ˜1 μM concertation of other prevalent enzymes. Such low concentrations and large differences in magnitudes pose a challenge for any amplification or statistically significant analysis using traditional sequencing tools.

To address these challenges, several recent efforts have been directed towards developing a new single-molecule sequencing method, using easily observable molecular fingerprints and a high-throughput and inexpensive technique. Optical sequence identification has emerged as an important candidate for a next-generation inexpensive and high-throughput sequencing technology and is potentially capable of identifying molecular sequences and variations in single molecules using their vibrational signatures. This approach also creates the potential for a single platform for combined proteomics, genomics, transcriptomics, and epigenomics. As such, there exists a need for a system for the optical sequence identification of single DNA, RNA and peptide molecules using individual SERS measurements and a molecular identification algorithm rooted in machine learning.

Building on the above described sequencing methods, in the push for precision medicine, there is an increasing demand for inexpensive, non-specific assays capable of broad-spectrum diagnostics, where a single test can rapidly screen an array of biomarkers. One immediate application of such a technology is to address the growing threat of antibiotic resistance, a public health crisis that affects nearly two million people in the U.S. annually. Rapid, affordable identification of drug-resistance in clinically relevant microbial strains is vital for prescribing patients with appropriate treatment plans to reduce mortality rates and the development of further resistances. Current resistance diagnostics and profiling assays are often performed only after initial antibiotics fail. Most of these assays rely on cell culturing, PCR amplification, and microarray analyses. Not only do these tests require hours to days and significant costs, but they are specific for detecting resistances of one or a few well-characterized strains. Next-generation, whole-genome sequencing approaches to resistance screening have shown promise; however, applications of this technology to diagnostics has been limited by lack of standardization protocols and the need for data interpretation leading to long diagnosis times.

A rapid, broad-spectrum diagnostic technique would also prove invaluable in the screening of cancers and other genetic diseases. Point-of-care diagnostic devices for sensitive and specific detection of cancer biomarkers have long been a goal of the bio-sensing community. Moreover, scientists and clinicians have long struggled to identify rare, novel, and undiagnosed disorders as evident by initiatives such as the National Institutes of Health (NIH) Undiagnosed Diseases Network. For cancers and other genetic diseases, early detection is crucial for patient survival. Current and emerging diagnostics continue to rely on the identification of the protein, peptide, or gene expression biomarkers. These diagnostic devices apply an array of nano-electronic and optical techniques, but like antibiotic resistance assays, are specific for detecting merely one or a few biomarkers for which the device is constructed.

As such, there exists a need for a novel and robust algorithmic platform, that may further be coupled with BOC technology as described below, to address the above identified shortcomings in the prior art. Such algorithms may provide a single, inexpensive diagnostic test capable of rapidly identifying a wide range of genetic biomarkers.

### SUMMARY OF THE INVENTION(S)

The inventive technology described herein includes optical systems and methods for accurately discriminating between different nucleobases or amino acids within single DNA, RNA, and protein molecules. The novel method utilizes a silver-coated silicon nanopillar substrate to trap individual biomolecules in SERS hotspots, allowing high-throughput single-molecule optical reads. Using spectroscopic ‘fingerprints’ that were identified from the spectral libraries that have been collected, the present inventors developed a novel molecular identification algorithm to accurately identify DNA and RNA bases, as well as a subset of naturally occurring amino acids. The optical nature of the measurement combined with the ability to trap and isolate single molecules on the substrate allows for the potential to simultaneously collect spectra from many hotspots on the same substrate using high-resolution optical microscopy, which provides a distinct advantage over other single-molecule sequencing methods that read molecules sequentially. (Background information related to certain embodiments related to the identification of polynucleotides by the applicant's novel BOC system may be included in co-owned U.S. Provisional Application No. 62/595,551, and U.S. Non-Provisional application Ser. No. 16/211,817. Notably, the entirety of that application's specification, including figures, related to earlier iterations of its BOS systems and identification of nucleotide content in a portion of a polynucleotide is incorporated herein by reference). By combining this approach with more sophisticated machine learning identification algorithms as generally described herein, it may be possible to deconvolute the contribution of different nucleobases or amino acids within the same spectrum, enabling accurate measurement of sequence content in mixed sequences. This novel approach to high-throughput (epi)genomics, transcriptomics, and proteomics at the level of single cells is generally described below.

The inventive technology described herein includes a comprehensive and robust algorithmic platform generally referred to as block optical content scoring (BOCS), generally referred to herein as the algorithm of BOC algorithm, that facilitates rapid, broad-spectrum genetic biomarker identification from DNA k-mer content. This algorithm builds upon novel systems and methods described below demonstrating the use of single-molecule Raman spectroscopy measurements for high-throughput, label-free detection of A-G-C-T content in DNA k-mers, called block optical sequencing (BOS). This BOS method is an alternative to single-letter sequencing and has the potential to simultaneously measure DNA k-mer content from millions of fragments simultaneously, thereby converting it into useful genetic information. This approach is akin to sharing and streaming of large multimedia files across the World Wide Web using a combination of lossless and lossy data compression techniques. The present inventor's bioinformatics approach, BOCS, uses the DNA k-mer content for identification of genetic biomarkers through probabilistic mapping of the k-mer content to gene databases. Comprehensive simulations show accurate and specific recognition of antibiotic resistance genes, as well as cancer and other genetic disease genes with less than full coverage of the genes and in the presence of sequencing error. The results described here for the BOCS algorithm system pave the way for a single, inexpensive diagnostic test capable of rapidly identifying a wide range of genetic biomarkers among other applications.

### BRIEF DESCRIPTION OF THE FIGURES

FIG. 1a-d shows SERS measurements of single-molecules and peak assignment. (a) Illustration of an exemplary polynucleotide molecule trapped in a SERS hotspot between two Ag-coated Si nanopillars. (b) Representative individual Raman spectra from solutions of poly-(dC)₅ DNA at concentrations of 0, 1.0 and 10 nM adsorbed onto nanopillar substrates. (c) Histogram of the estimated occupancy (number of molecules) in SERS measurements of samples prepared by adsorption of 10.0 nM poly-(dC)₅ DNA onto the nanopillar substrates. Overlaid onto the histogram is the best-fit Poisson distribution to the data. (d) Examples of individual Raman spectra collected from poly-(dC)₁₀₀ DNA on the nanopillar substrates, showing different groups of peaks present in different spectra. The vertical bars indicate positions of characteristic cytosine peaks (green), as well as background peaks (gray).

FIG. 2a-d shows molecular identification algorithm for optical sequence identification. (a) Table of Raman peak centers and FWHM values (cm⁻¹) from Gaussian fitting to peaks of interest in the average spectra. The highlighted peaks were used by the DNA/RNA molecular identification algorithm. (b) First, a Gaussian distribution is fit to each peak within a representative average spectrum for each class and selected optimal peaks (red) are chosen to produce an optical fingerprint for that class. (c) Each unknown measurement is compared to the peak position and FWHM values for peaks in the fingerprint of the class and used to estimate the probability that the measurement belongs to that class. (d) This process is repeated for each possible class, and (e) the class with the highest probability is chosen to make a base call.

FIG. 3a-d shows metrics for optical sequence identification: Confusion matrices and sequencing trace plots from DNA/RNA base calling. Shown on the left are confusion matrices that plot the fraction of measurements predicted for each class vs. their actual class, for (a) five DNA nucleobases and (b) four RNA nucleobases. Shown on the right are representative segments of sequence trace plots resulting from applying our molecular identification algorithm to randomly generated ‘unknown’ sequences of (c) DNA and (d) RNA bases. The actual sequence is shown with an ‘X’ marked above incorrectly classified bases. The plots also show the calculated probability of each class, as well as the resulting confidence for each classification. The modified nucleobase 5-methylcytosine is represented in the DNA trace plot by C*. Full sequence trace plots are shown in FIG. 11.

FIG. 4a-b shows Raman Peak fitting and molecular identification for the four selected amino acids. (a) Table of Raman peak centers and FWHM values (cm⁻¹) from Gaussian fitting to peaks of interest in the average spectra. The highlighted peaks were used by the amino acid molecular identification algorithm. (b) Confusion matrix plotting the fraction of measurements predicted for each class vs. the actual class for the four amino acids tested.

FIG. 5 shows a schematic for the BOCS algorithm. The BOCS algorithm takes measurements of DNA k-mer content from high-throughput single-molecule Raman spectroscopy measurements and maps them to gene databases for probabilistic determination of genetic biomarkers at low coverages. Starting with a log of measured k-mer content blocks (B1 . . . Bn as shown) and a genetic biomarker database (excerpts from the MEGARes antibiotic resistance database are shown), the blocks are individually aligned to each gene in the database based on content. This alignment consists of finding all match locations for the k-mer block content within a gene via translating through the gene one nucleotide at a time and looking at fragments of length k. For each block, a raw probability can be calculated for each gene based on the number of matches for the k-mer block content within the gene, length of the k-mer block, and length of the gene (calculation shown in the schematic). As more blocks are analyzed, probabilities are compounded and genes in the database are ranked. The gene(s) from which the Raman-analyzed k-mer blocks originate quickly generate the top probabilities and can often be determined in coverages <<1.0, meaning that only a small fraction of the gene blocks need to be analyzed for identification of a specific genetic biomarker.

FIG. 6a-e shows the rapid identification of antibiotic resistance genes. 70 randomly-selected antibiotic resistance genes from the MEGARes database were each run through the BOCS simulation with 25 repeats, for a total of 1750 simulations. Results are shown for both the cases of no thresholding and entropy screening (red) and with thresholding and entropy screening (blue). (A) Histogram of the coverage at which a resistance gene is identified combined for all 70-gene simulations. Details of the average coverage and accuracy are given in the inset. Results demonstrate that most genes can be identified with 100% accuracy at merely 0.15-0.30 coverage of the gene. With thresholding and entropy screening, the average coverage decreased and led to more specific gene identifications. (B) Specificity with increasing coverage for all compiled 70-gene simulations. It is demonstrated that about 90% of the genes in the database can be eliminated at coverages as low as 0.10. With thresholding and entropy screening, more genes are eliminated at lower coverages leading to higher specificity in the identification process. (C) Histograms of the average coverage at which a resistance gene is identified for four individual genes (gene labels shown at the top). The histograms show a clear shift towards lower coverages for the thresholding and entropy screening case (data from the 25 simulations for each case are shown). (D) Specificity with increasing coverage for the four individual genes. Dots indicate the average locations at which a gene is identified. Again, significant shifts towards lower coverages are seen in the case with thresholding and entropy screening. (E) Increasing content scores with coverage for four individual genes. The selected genes are colored blue/red for cases with/without thresholding and entropy screening. The grayed lines are all other genes in the resistance gene database (3823 of the 3824 total). As coverage increases (i.e., as more blocks are analyzed), the selected genes quickly separate themselves from the others probabilistically, leading to their identification at low coverages. The separation happens sooner, and more significantly, in the case of thresholding and entropy screening.

FIG. 7a-i shows antibiotic resistance gene identification with sequencing variability. (A, B, C) The effect on accuracy, coverage for identification, and false positives as k-mer length is varied. For values of k=8, 10, and 12, all blocks are set to length k. For the ‘Variable’ mode, block lengths are sampled from a normal distribution centered around k=10, leading to a distribution of block lengths from ˜6-14. Accuracy, coverage for identification, and false positive rate are all weakly dependent upon k-mer length. For all k-mer trials, the accuracy remains >99%, coverage remains <0.40, and false positives remain <<1. (D, E, F) The effect on accuracy, coverage for identification, and false positives with errors in the blocks. Even at 20% error rates, the average accuracy remains >90%, the coverage for identification never reaches 1.0, and false positives are low. (G, H, I) The effect on accuracy, coverage for identification, and false positives as blocks from multiple genes are analyzed. Accuracy decreases linearly with an increasing number of genes in the analysis, but remains near 80% for five genes, with average coverage of around 0.60. The main hindrance with an increasing number of genes is the large false positive rate. For the k-mer length and errors analyses in parts A-F, each data point on the graphs is a result of 70 randomly-selected antibiotic resistance genes from the MEGARes database each run through the BOCS simulation with 25 repeats, for a total of 1750 simulations. For the multiple genes analysis in parts G-I, the 2-gene and 5-gene results are from 10 random 2-gene selections and 5 random 5-gene selections from the base set of 70 randomly-selected antibiotic resistance genes, each with 25 repeats.

FIG. 8a-c MRSA detection with BOCS. A BOCS simulation was set up to test the viability of detecting a generic MRSA strain on the basis of two resistance genes (a class D beta-lactamase OXA gene and a mecA gene for the penicillin-binding protein PBP2a), which are the norm for both phenotypic and non-phenotypic diagnostic methods. The simulation also included sequencing inconsistencies in the form of variable k-mer block lengths centered around k=10 and a 4% error rate within the blocks. 50 repeat simulations were run for the statistics presented. (A) Histogram of the coverage at which the resistance genes are identified in each of the 50 repeat simulations. (B) Specificity with increasing coverage. Dots indicate the average coverages at which the OXA and mecA genes were identified. The lag where specificity remains at zero during low coverages is a result of a high thresholding multiplier, which was set at 15. (C) Increasing content scores for the OXA and mecA genes with coverage. The grayed lines are all other genes in the resistance gene database (3822 of the 3824 total). As coverage increases (i.e., as more blocks are analyzed), the genes of interest quickly separate themselves from the others probabilistically, leading to MRSA detection at low coverages.

FIG. 9a-d shows BOCS applied to other genetic biomarkers. To demonstrate the versatility of the BOCS algorithm, simulations were run for identifying single genes from databases for cancer genes (COSMIC database) and other genetic diseases (custom compiled database—see the Supplementary Information for more details). For each database, 10 randomly-selected genes were run with 10 repeats, for 100 total simulations. (A, B) Histogram of the coverage at which the cancer genes are identified and the specificity with increasing coverage for the cancer genes detection. Accuracy is 100% with an average identification coverage of 0.34, and about 90% of the 29360 genes are eliminated after merely 0.10 coverage. (C, D) Histogram of the coverage at which the genetic disease genes are identified and the specificity with increasing coverage for the genetic disease genes detection. Accuracy is 100% with an average identification coverage of only 0.132, and about 95% of the 256 genes are eliminated after just 0.10 coverage.

FIG. 10a-f shows example Raman spectra from DNA & RNA nucleobases. Shown are representative examples of individual Raman spectra (a-f) collected from poly-(dN)ₓ and poly-(rN)ₓ homopolymers (N=A, G, C, T, U, or 5 mC) on the nanopillar substrates, showing different groups of peaks present in different spectra. The vertical bars indicate positions and widths of characteristic peaks (green), as well as background peaks (gray).

FIG. 11a-b shows full DNA/RNA sequence identification trace plots. Shown are the full sequence trace plots from applying the molecular identification algorithm to randomly generated ‘unknown’ sequences of (a) DNA and (b) RNA bases. The actual sequence is shown with an ‘X’ marked above incorrectly classified bases. The plots also show the calculated probability of each class, as well as the resulting confidence for each classification. The modified nucleobase 5-methylcytosine is represented in the DNA trace plot by C*.

FIG. 12a-d shows example Raman spectra from amino acids. Shown are representative examples of individual Raman spectra (a-d) collected from poly-(X)₅ polypeptides (X=His, Met, Ser, Tyr) on the nanopillar substrates, showing different groups of peaks present in different spectra. The vertical bars indicate positions and widths of characteristic peaks (green), as well as background peaks (gray).

FIG. 13a-f shows DNA surface-enhanced Raman signal from positively-charged silver nanoparticles. (a-d) Raman signatures with marked signature peaks, *, for (a) A—adenine, (b) G—guanine, (c) C—cytosine, and (d) T—thymine. All signature data was collected from homologous 10-mer length DNA fragments at 10 nM DNA concentrations. Raman signal has been baseline subtracted and normalized to the peak near ˜1090 cm⁻¹, which corresponds to the DNA phosphate backbone. (e) Matrix analysis of raw intensity measurements extracted from the Raman signatures (baseline subtracted and normalized signatures as shown in parts a-d) for A, G, C, and T. Each row represents each nucleobase, and each column represents each signature peak. (f) Significance test in the form of p-values for the nucleobase signatures. The p-values were calculated from a two-sample t-test assuming equal variance with intensity values down each column from part e. The signature peaks for each nucleobase are confirmed to be significant at p<0.05 levels.

FIG. 14 shows DNA epigenetic modifications. Comparing the Raman signal of cytosine (C) with 5-methylcytosine (5 mC), differences can be seen in the marked regions around ˜600 cm′ and ˜800 cm⁻¹, which correspond to the signature ring bending mode for C and the ring breathing mode for C/T, respectively. Data was collected from homologous 5-mer length DNA fragments of C and 5 mC at 10 nM DNA concentrations. Raman signal has been baseline subtracted and normalized to the peak near 1090 cm⁻¹, which corresponds to the DNA phosphate backbone.

FIG. 15a-f shows RNA surface-enhanced Raman signal from positively-charged silver nanoparticles. (a-d) Raman signatures with marked signature peaks, *, for (a) A—adenine, (b) G—guanine, (c) C—cytosine, and (d) U—uracil. All signature data was collected from homologous 7-mer length RNA fragments at 10 nM RNA concentrations. Raman signal has been baseline subtracted and normalized to the peak near 1090 cm⁻¹, which corresponds to the RNA phosphate backbone. (e) Matrix analysis of raw intensity measurements extracted from the Raman signatures (baseline subtracted and normalized signatures as shown in parts a-d) for A, G, C, and U. Each row represents each nucleobase, and each column represents each signature peak. For U, the signature is a combination of a dual C/U peak at ˜800 cm⁻¹ and the lack of significant C peak at ˜590 cm⁻¹; therefore, the last column is shown for the combined C/U peak. (f) Significance test in the form of p-values for the nucleobase signatures. The p-values were calculated from a two-sample t-test assuming equal variance with intensity values down each column from part e. For the U signature, p-values were generated with a χ² analysis according to Fisher's method due to the two-peak signature. The signature peaks for each nucleobase are confirmed to be significant at p<0.03 levels.

FIG. 16a-d shows comparison of the DNA and RNA Raman signals. (a-d) Similar features are observed in respective DNA/RNA signatures for each nucleobase (a) A—adenine, (b) G—guanine, (c) C—cytosine, and (d) T/U—thymine/uracil. A new feature characteristic of all RNA measurements appears near ˜430 cm⁻¹. Data was collected from homologous 10- and 7-mer length DNA and RNA fragments at 10 nM DNA and RNA concentrations. Raman signal has been baseline subtracted and normalized to the peak near ˜1090 cm⁻¹, which corresponds to the DNA and RNA phosphate backbone.

FIG. 17a-c shows amino acid surface-enhanced Raman signal from positively-charged silver nanoparticles. (a-c) Unique Raman signatures can be seen for three amino acids (a) His—histidine, (b) Met—methionine, and (c) Tyr—tyrosine. For amino acids, a magnesium sulfate aggregating agent is needed. The common Raman peak near ˜985 cm⁻¹ is from the magnesium sulfate. Data was collected from homologous 5-mer length peptide fragments at 100 nM peptide concentrations. Raman signal has been baseline subtracted.

FIG. 18 shows amino acid phosphorylation. Comparing the Raman signal of tyrosine (black line) with phosphorylated tyrosine (red line), differences can be seen in the marked region from ˜700-750 cm′. Data was collected from homologous 5- and 2-mer length peptide fragments at 100 nM peptide concentrations. Raman signal has been baseline subtracted.

FIG. 19a-e shows and overview of the proposed optical sequencing method with positively charged silver nanoparticles. (a) SERS measurements of ssDNA kmer blocks are collected from colloidal suspensions of positively charged Ag NPs with a 532 nm laser. Signal enhancement is achieved via aggregation of the Ag NPs in the presence of negatively charged DNA k-mer blocks, as evident by the red-shift in the extinction spectrum. (b) Raman signatures for the four DNA nucleobases A, G, C, and T collected from homologous 10-mer sequences. The signatures provide the most distinctive Raman mode peaks for each base, which are used to deconvolute the content of mixed sequence k-mer blocks. These “signature peaks” are marked along with the 1089 cm⁻¹ PO₂ str. peak used for normalization (A: ˜740 cm⁻¹ ring br., G: ˜690 cm-1 ring br., C: ˜600 cm⁻¹ ring bend, T: ˜460 cm⁻¹ ring bend). (c) In mixed sequence DNA blocks, the four signature peaks are present with relative intensities (normalized to the PO₂⁻ peak) corresponding to their respective content. (d) Raman signal for RNA and DNA show near-identical shifts (shown for adenine, A, in RNA and DNA), demonstrating the potential for transcriptomic analyses. (e) Subtle perturbations are seen in the Raman signal due to nucleobase chemical modifications (shown and highlighted for the cytosine, C, modification to 5-methylcytosine, 5 mC), demonstrating the potential for epigenomic studies.

FIG. 20a-d shows calibration measurements. Analyzing the correlations between varying nucleobase content within the DNA 10-mer calibration blocks from Table 5 and changes in the signature peak intensity for (a) A: ˜740 cm⁻¹ ring br., (b) G: ˜690 cm⁻¹ ring br., (c) C: ˜600 cm-1 ring bend, and (d) T: ˜460 cm⁻¹ ring bend. (Left) For each nucleobase, increasing content within a block (lighter to darker shades, labeled in the plot legend with the corresponding calibration 10-mer block from Table 5) leads to a linear increase in the intensity of the signature peak. (Right) Linear fits, with the intercept locked at zero, of the measured signature peak normalized intensity versus content within the block (data points and variance are from five technical replicates of each calibration block). These fits are used as calibrations to identify the content in an unknown mixed sequence of DNA k-mer blocks.

FIG. 21 shows content identification within gene blocks. The content of unknown mixed sequence DNA blocks (shown for the 15 10-mer gene blocks from Table 6) can be identified from the calibrations for each of the four nucleobases. Using block Gen_4 as an example, the measured normalized intensity for each of the four signature peaks (averaged from three technical replicates) is used to predict the raw content. This raw content is then normalized such that the total predicted content equates to one. The normalized content is then rounded so that each base has an integer number within the block.

FIG. 22a-b show highly accurate content identification. Actual and predicted content is compared for the 15 10-mer gene blocks from Table 6. Since optical sequencing relies on the content and not letter-by-letter sequences, one misidentification results in a double error because the contents of the incorrect nucleobase and substituted nucleobase are both affected. In the figure table, correct predictions are highlighted in green and incorrect predictions are highlighted in red. A confusion matrix analysis on the single nucleobase level shows that the majority of errors result from guanine, G, content being under identified (˜10% of G bases throughout the gene blocks). In total, the content for the 15 gene blocks was identified at an average accuracy of 93.3%.

FIG. 23a-c show MDR pathogen profiling with optical sequencing. (a) Overview of the content-scoring algorithm integration with optical sequencing measurements. Starting with a log of measured content within DNA k-mer blocks (B1 . . . Bn as shown) and a gene database (excerpts from the MEGARes antibiotic resistance database are shown), the blocks are individually aligned to each gene in the database based on the content. This alignment consists of finding all match locations for the k-mer block content within a gene via translating through the gene one nucleotide at a time and looking at fragments of length k. For each block, a content score is calculated based on the number of matches for the k-mer block and various probability factors. As more blocks are analyzed, content scores are compounded and genes in the database are ranked and eliminated. The algorithm was run for the 15 10-mer gene blocks in Table 6 from an OXA β-lactamase gene (with the predicted content at 93.3% accuracy). Note that only 12 of the 15 blocks were used, as three were eliminated with entropy screening. Two cases were studied: 1. Identifying the gene from the MEGARes antibiotic resistance database of ˜4000 resistance genes (b) and 6. Identifying the gene within the P. aeruginosa genome (c). Both cases demonstrate the robust identification of the correct OXA resistance gene from content score ranking, requiring merely a few content measurements. Additionally, >90% of genes in both databases were eliminated after a single block was analyzed by the content-scoring algorithm. The following settings were used when running the software/algorithm:penalty score: 0.1, thresholding multiplier: 0.1, entropy screening: “on” (eliminated only the blocks with permutations >25 000).

Supplementary Tables 1-16 show supplementary information tables of detailed results for the figures presented herein. This includes information on all of the individual genes used in the enabling simulations, as well as full simulation results for single-gene studies with and without entropy screening, varying k-mer lengths, and block errors; multiple-gene studies; and cancer and other genetic disease results. Supplementary information tables include:

### DETAILED DESCRIPTION OF THE INVENTION

Described herein are devices, techniques, and systems that employ multiplexed 3D plasmonic nanofocusing, optical signatures from nanometer-scale mode volumes to aid in identifying macromolecules, and in particular DNA, RNA and polypeptides. In one preferred embodiment, the inventive technology includes devices, methods, and systems for rapid and high throughput sequencing of macromolecules, such as proteins using optical methods to identify the amino acid content of a block of a polypeptide. The disclosed methods may include an inherent lossy compression of proteomic information, which can be used to rapidly identify specific target sequences, modifications, mutations, alternative splicing and the like, as well as provide protein sequence information. In one embodiment, the disclosed methods and systems combine Raman spectroscopy with other optical methods, such as FTIR to help increase the sensitivity and accuracy of fingerprinting as well as sequencing.

For example, as described herein, is the use of Raman spectroscopy and FTIR spectroscopy for label-free identification of protein amino acids, as well as RNA and DNA nucleobases. The disclosed method identifies characteristic molecular vibrations using optical spectroscopy, especially using the “fingerprinting region” for different molecules from ˜400-1400 cm′, to determine, in one embodiment, the amino acid content of a block, or portion, of a polypeptide, as well as. These block fingerprints can then be analyzed and compared with other block fingerprints to identify a specific target polypeptide or protein sequence.

In one preferred embodiment, the invention may include Described herein are devices, techniques, and systems that employ multiplexed 3D plasmonic nanofocusing, optical signatures from nanometer-scale mode volumes to aid in identifying amino acid content in peptide k-mer blocks. The content of each amino acid in a block can be used as a unique and high-throughput method for identifying sequences, mutations, and other markers as an alternative to single-letter peptide sequencing. Here, surface-enhanced Raman spectroscopy is used for label-free identification of protein amino acids, as well as DNA and other RNA nucleobases, with multiplexed 3D plasmonic nanofocusing. It is shown that the content of each amino acid in a peptide block can be used as a unique and high-throughput method for identifying sequences, mutations, and other markers as an alternative to single letter peptide sequencing. Additionally, it is shown that coupling two complementary vibrational spectroscopy techniques (infrared and Raman spectroscopy) can improve block characterization. These results can pave the way for the development of a novel, high-throughput block optical sequencing method with lossy genomic and/or proteomic data compression using k-mer identification from multiplexed optical data acquisition.

The described devices, processes, and systems are useful in label-free, high-throughput block optical sequencing (BOS) with inherent lossy compression. In many of these embodiments, k-mer blocks of peptides are read using 3D nanofocusing of light. Since the different amono acid based in peptides are biochemically distinct, their unique interactions with light photons (observable optical fingerprints) can be used to discriminate them. Surface-enhanced Raman spectroscopy (SERS) is an optical method routinely used for identification of unknown chemical and biochemical compounds from their vibrational fingerprints. In this technique, surface plasmon polaritons lead to 3D nanofocusing and enhancement of near field signal at the apex of rough features or patterned nanostructures. However, applying SERS, or the related tip-enhanced Raman spectroscopy (TERS), for reproducible single-molecule molecules, such as DNA sequence identification has proven difficult. Previous studies have used SERS/TERS measurements on DNA for label-free chemical fingerprinting; however, mixing of a large number of DNA molecules with metal nanoparticles provides an ensemble spectra and poses uncertainties in signal strengths. Furthermore, small molecules, such as polypeptides have varied enhancement due to differences in their location from the plasmonic antenna, and thus suffer from low reproducibility. Since the SERS/TERS signal falls off dramatically with distance from the plasmonic antenna, it makes signal amplitudes highly sensitive to the orientation and conformation of molecules with respect to the surface. While many of these effects are washed out in an ensemble detection, it has been shown that the SERS/TERS signal strength and reproducibility are severely affected by the packing fraction and large uncontrollable variation in molecular orientation with respect to the plasmonic nanostructure. Thus, single-molecule label-free identification of amino acids remains an important and critical challenge.

As such, in certain embodiments described herein is the use of patterned nanopyramid probes on a multiplexed substrate to reproducibly enhance “optical fingerprints” of peptide amino acids. Identifying the different molecular vibrations, bond stretches, and rocking motions in these reproducible spectra allowed differentiation of the amino acids peptide bases from their respective spectral fingerprints. In addition, the disclosed identification techniques may be improved by combining Raman with Fourier-transform infrared (FTIR) spectroscopy.

Probes for use with the disclosed methods and techniques may be fabricated using methods known to those of skill in the art to obtain a suitable shape for providing Raman scatter or FTIR absorbance information from a polypeptide. In some embodiments, the probes may be manufactured with a pyramidal shape of three or four sides, such that they end in a tip with significantly reduced surface area relative the base of the shape. In other embodiments, the shape may be other than pyramidal, for example square, conical, or cylindrical.

In many embodiments, nanopyramidal probes may be fabricated from various compositions. In some embodiments, metal pyramids are used. In one embodiment, the periodicity of the nanopyramids may be about 2 μm and in various suitable patterns. For example, as described below, a square periodic pattern may be used with 2 μm periodicity in both the x and y direction. In many embodiments, this may help enhance vibrational signal using the fingerprinting region of the mid-IR region. Probes may have characteristics that help to retain a polypeptide at the tip. In some embodiments, the composition of the material at the tip of the probe may have a charge that is opposite of the polypeptide to aid in retaining the polypeptide, for example the tip may be positively charged to attract and retain negatively charged polypeptides. In some embodiments, other surfaces of the tip may be of a material that may repel or poorly interact with a polypeptide.

Probes for use with the disclosed methods and techniques may define a surface for accepting or interrogating a polypeptide. In some embodiments, the surface of the probe may be a tip of the probe that may be blunt or sharp. A blunt tip may define a surface that can accommodate a polypeptide of 1 to about 10 nm. In many embodiments, the polypeptide being interrogated may be longer than the surface of the tip. In some embodiments, the tip may have a have a diameter of about 1 to 10 nm, or about 2-7 nm, or about 2 nm, 3 nm, 4 nm, or 5 nm. In many embodiments, the tip may be designed to interrogate a portion or block of a polypeptide that is from about 2 to about 20 nt. In other embodiments, the tip may be designed to interrogate 3 nt to about 10 nt.

A surface for use with the disclosed devices, methods, techniques, and systems may have a plurality of probes. In some embodiments, a surface may have about 1×105 to about 1×1010 probes, for example 1×106 or 1×109 probes. In many embodiments, a plurality of probes may be analyzed simultaneously or sequentially for Raman scatter and FTIR for, in one preferred embodiment amino acid content of a polypeptide positioned on the tip of the probe.

Laser light may be directed at one or more probes to interrogate a polypeptide at, on, or near a tip of the probe. Light reflected from the portion of the polypeptide at the tip may be analyzed by various spectrophotometric methods. In some embodiments, scattered light is analyzed by a Raman spectrophotometer. In some embodiments, absorbance may be analyzed by FTIR spectrophotometer. In some embodiments, one or more filters may be used to analyze light within the wavenumber range.

The polypeptide may be applied to the surface, for example the probe tip by various methods. In most embodiments, wherein the portion of the polypeptide is interrogated on a probe tip, the tip may support or be in contact with a single polypeptide. In some embodiments, the polypeptide may be combed on the surface so that it is substantially linear.

The polypeptide may be treated prior to applying it to the surface. In one embodiment the polypeptide is digested or fragmented by enzyme or chemical treatment, for example with a specific protease enzyme. In some embodiments, the fragmentation may provide a fragment size that is similar to, but generally larger, than that of the block size being analyzed. A portion, or block, of a polypeptide may be analyzed by the described method. In some embodiments, the block may comprise from about 2 to about 20 amino acids, for example 2, 3, 4, 5, 6, 7, 8, 9, 10, 11, 12, 13, 14, 15, 16, 17, 18, 19, or 20 amino acids. The number of amino acids in a block may be referred to as the “k” number. In most embodiments, a polypeptide comprises a plurality of blocks.

The disclosed methods, techniques, devices, and systems are useful in determining the amino acid composition of an interrogated block. In some embodiments, the disclosed methods may be useful in determining the relative or absolute number of each type of amino acid in a block. In many embodiments, this composition of a given block may represent a fingerprint for that block.

The disclosed methods and techniques for identification and sequencing of polypeptide may represent lossy compression. In the disclosed techniques and methods, the identity and order of amino acids within a given block is not determinable by analysis of the light from that tip. In some embodiments, fingerprints of multiple blocks at multiple tips may be combined to provide an overall sequence of a given amino acids comprised of the analyzed blocks.

As noted herein, while in certain embodiment the inventive technology has been described to the identification of polypeptides; such applications may also be applied to the identification of polynucleotides or amino acids as generally described herein.

The disclosed devices, methods, techniques, and systems may be used to sequence a plurality of polynucleotides or polypeptide by movement of the probe tip relative to the polynucleotide or polypeptide. In this embodiment, the polynucleotide or polypeptide may be applied to a surface other than a probe tip, and then a probe tip may be moved into proximity with the polynucleotide or polypeptide. When the tip is moved along the polynucleotide or polypeptide, the fingerprint will change as one nucleotide or amino acid at the end of the block is lost, and a new nucleotide or amino acid is added to the beginning of the block.

Additional embodiments of the current inventions include a single, inexpensive diagnostic test capable of rapidly identifying a wide range of genetic biomarkers would prove invaluable in precision medicine. Previous work has demonstrated the potential for high-throughput, label-free detection of A-G-C-T content in DNA k-mers, providing an alternative to single-letter sequencing while also having inherent lossy data compression and massively parallel data acquisition. Here, the present inventors apply a new bioinformatics algorithm—block optical content scoring (BOCS)—capable of using the high-throughput content k-mers for rapid, broad-spectrum identification of genetic biomarkers. BOCS uses content-based sequence alignment for probabilistic mapping of k-mer contents to gene sequences within a biomarker database, resulting in a probability ranking of genes on a content score. Enabling simulations of the BOCS algorithm reveal high accuracy for identification of single antibiotic resistance genes, even in the presence of significant sequencing errors (100% accuracy for no sequencing errors, and >90% accuracy for sequencing errors at 20%), and at well below full coverage of the genes. Simulations for detecting multiple resistance genes within a methicillin-resistant Staphylococcus aureus (MRSA) strain showed 100% accuracy at an average gene coverage of merely 0.416, when the k-mer lengths were variable and with 4% sequencing error within the k-mer blocks. Extension of BOCS to cancer and other genetic diseases met or exceeded the results for resistance genes. Combined with a high-throughput content-based sequencing technique, the BOCS algorithm potentiates a test capable of rapid diagnosis and profiling of genetic biomarkers ranging from antibiotic resistance to cancer and other genetic diseases.

The BOCS algorithm uses content-based alignment for probabilistic mapping of k-mer contents to gene sequences within a biomarker database. The algorithm applies elements from pattern recognition and machine learning to rank biomarkers based on a content score. Simulations of the BOCS algorithm showed 100% accurate and highly-specific identification of single antibiotic resistance genes at average coverages of merely 0.255±0.096. Further simulations demonstrated robust performance of the BOCS algorithm in the presence of variable k-mer lengths and high sequencing error rates. With errors as high as 20%, over 90% accuracy in gene identification was achieved at less than full gene coverages.

Additionally, BOCS has the ability to identify multiple genes when the k-mer fragments from the multiple genes are randomly mixed. When applied to a clinically relevant MDR bacterial strain, the BOCS algorithm showed 100% accuracy with a low false positive rate for detection of two resistance genes (mecA and OXA for MRSA identification) at an average coverage of 0.416±0.296, with a block error rate of 4% and variable k-mer lengths. BOCS applied to cancer and other genetic diseases also showed detection at 100% accuracy with coverages at or below the values for resistance genes. When coupled with a high-throughput content-based sequencing platform, the BOCS algorithm can provide a biomarker detection tool applicable for rapid, broad-spectrum diagnostics.

As noted above, the disclosed BOCS algorithm, methods, techniques, and systems may be implemented in a digital computer system. Such a digital computer is well-known in the art and may include one or more of a central processing unit, one or more of memory and/or storage, one or more input devices, one or more output devices, one or more communications interfaces, and a data bus. In some embodiments, the memory may be RAM, ROM, hard disk, optical drives, removable drives, etc. In some embodiments, storage may also be included in the disclosed system. In some embodiments, storage may resemble memory that may be remotely integrated into the system. The input and output devices may be, for example one or more monitors, display units, video hardware, printers, speakers, lasers, spectrophotometers, filters, collectors, cameras, etc.

### EXAMPLES

### Example 1: Single Molecule SERS Measurements on Leaning Nanopillar Substrates

Optical sequencing of amino acids and nucleotides in proteins, DNA, and RNA from individual cells requires a strong enhancement of the optical signatures in order to accurately detect and characterize the signal from single molecules. Furthermore, individual proteins or nucleic acid molecules must be spatially isolated on a substrate such that their respective signals can be resolved. To achieve reproducible and high-density SERS enhancement on an inexpensive substrate, the present inventors used ‘leaning nanopillar’ substrates that were generated by reactive ion etching of silicon wafers followed by deposition of a thin coating of silver metal. These substrates, which can be generated in wafer scale and are commercially available, trap single-molecules in nanoscale ‘hotspots’ that focus and intensify the local electromagnetic field, resulting in an easily observable optical signal enhanced by many orders of magnitude over the signals from molecules in the surrounding regions.

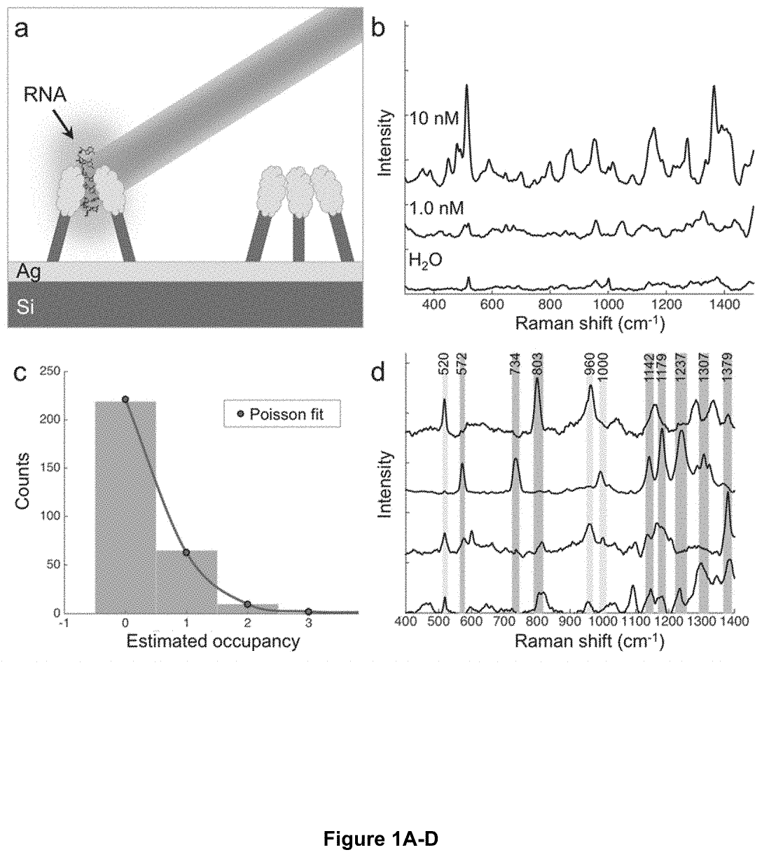

As illustrated in FIG. 1a, the molecules are adsorbed onto the substrate from a small droplet (˜0.1 μL) of a dilute (˜1-10 nM) aqueous solution, which is then allowed to evaporate completely. As the solution evaporates, the surface tension at the air/liquid/solid interface of the receding droplet causes the pillars to lean into one another (FIG. 1a), trapping molecules that are adsorbed near the tops of the pillars in hotspots with Raman enhancement factors of up to ˜10¹¹. In the case of bio-macromolecules like proteins, DNA, and RNA, there will be part, or all of the chain trapped in a given hotspot, leading to an optical signal that is a mixture of the signals from the different constituent monomers (nucleotides or amino acids). Thus, each Raman spectrum encodes the sequence content of the molecule as a convolution of spectra from the individual monomers. This can be used to determine the relative amounts of each of the monomers present in a given molecule by using the relative intensity of fingerprint peaks that have been identified for each monomer, as was shown previously for DNA on a different SERS substrate. These measurements sequence the ‘blocks’ (also known as block optical sequencing, BOS), which can be combined with computational methods to uniquely identify genes using a minimal number (˜5-10) of blocks to enable high-throughput genomic or transcriptomics profiling of individual cells.

In order to test the viability of using the leaning nanopillar substrates for identifying biomolecule sequence content from Raman spectra, the present inventors first carried out SERS measurements on short poly-(dC)₅ DNA homopolymers adsorbed from solution droplets with varying DNA concentrations. To do this, water droplets containing DNA concentrations of 0, 1.0, 10, and 100 nM were deposited onto the substrate and allowed to dry. Then several hundred Raman measurements were acquired pointwise along a grid within the droplet area, with a grid point spacing of approximately 10 Examples of resulting spectra are shown in FIG. 1b. The blank control (water only) displays a few peaks that must be assigned given their presence in all spectra. The broad band at around 230-240 cm⁻¹ has been previously observed in SERS measurements on nanostructured silver and can be attributed to Ag—O vibrational modes. The sharp peak at 520 cm⁻¹ corresponds to the well-known Raman band of the underlying crystalline silicon substrate. Likewise, the pair of bands at 960 and 1000 cm⁻¹ have been previously observed on similar substrates and are also considered to be background peaks. When the measurements were repeated with a water droplet containing 1.0 nM poly-(dC)₅ DNA, the majority of spectra resemble those collected with the blank control, indicating that no molecules were trapped within the measured areas. However, a small fraction of the spectra contain a new set of peaks that are characteristic of the cytosine nucleotide.

When the DNA concentration was increased to 10 nM, the fraction of spectra showing significant peaks from cytosine increased to ˜20%, with a few measurements even showing DNA peaks with roughly twice the intensity relative to background peaks, indicating an increase in the number of molecules trapped in SERS hotspots per unit area. Further increase of the DNA concentration to 100 nM resulted in a larger fraction of the spectra showing significant DNA peaks; however, many spectra also displayed a very high intensity relative to the background, indicating that most measurements now contained multiple DNA molecules trapped in hotspots. To identify optical fingerprints from measurements on single molecules, the present inventors carried out all further measurements using a concentration of 10 nM, as it provides a good balance between minimizing the chances of measuring multiple molecules in a given spectrum and reducing the required number of raw measurements to achieve a statistically relevant sample size.

To further confirm that the collected Raman spectra do indeed arise from SERS signals of individual molecules, the present inventors next sought to use the relative intensity of the peaks in each measurement to estimate the number of molecules trapped in hotspots, or occupancy, for that measurement. To accomplish this, the present inventors first took the scaled average of the spectra that displayed significant non-background peaks and determined the vibrational mode to which each peak corresponds using peak positions previously reported in the literature. Spectra that did not display significant non-background peaks were considered to have no molecules trapped in hotspots within the measurement area (occupancy=0) and were not included in the following analysis. Of the remaining measurements, the present inventors calculated the median absolute deviation (MAD) of peak intensity for each peak in order to find the expected peak intensity range for single occupancy, assuming that multiple occupancy is relatively rare. Then, for each peak within a given spectrum, the ‘peak occupancy’ was determined by comparing the peak intensity to the MAD for that peak. The estimated occupancy for that spectrum was then taken as the largest peak occupancy. The results were then fit to a Poisson distribution using the following equation:

$$P ( k ) = {e}^{- λ} \frac{{λ}^{k}}{k !}$$

where k is the occupancy number, λ, is the mean, and P(k) is the probability of having occupancy k in a given measurement. The resulting occupancy histogram and the Poisson fit are shown in FIG. 1c. The close agreement between the histogram and the Poisson fit supports the presence of a discrete number of molecules in each measurement, while the resulting lambda value of λ=0.28 confirms that the majority of measurements correspond to either 0 or 1 molecule trapped in a SERS hotspot.

### Example 2: Raman Fingerprinting for Nucleic Acid Identification

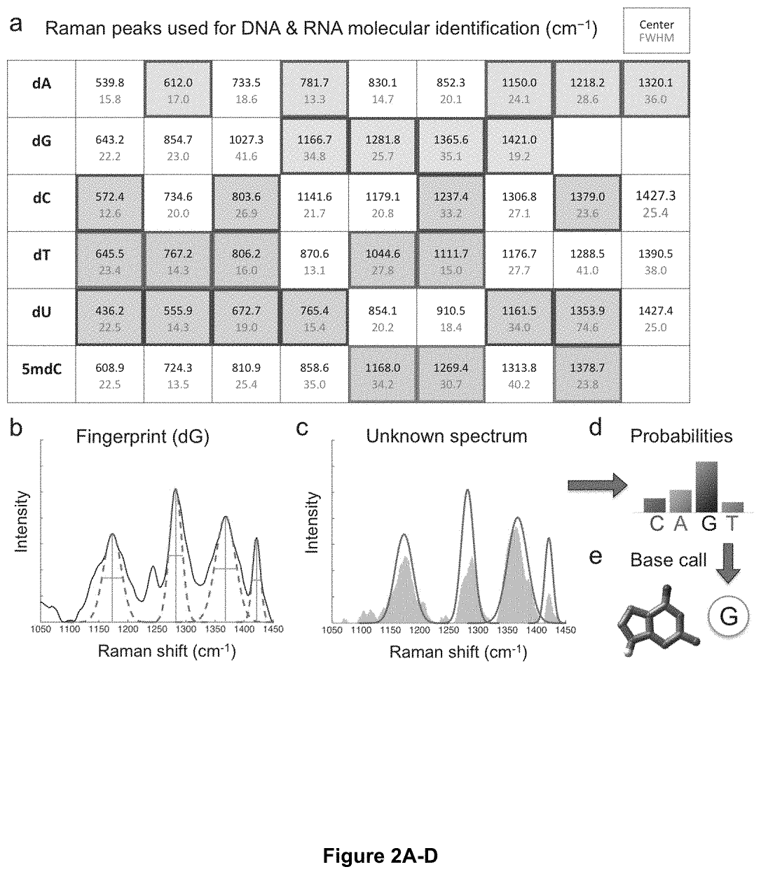

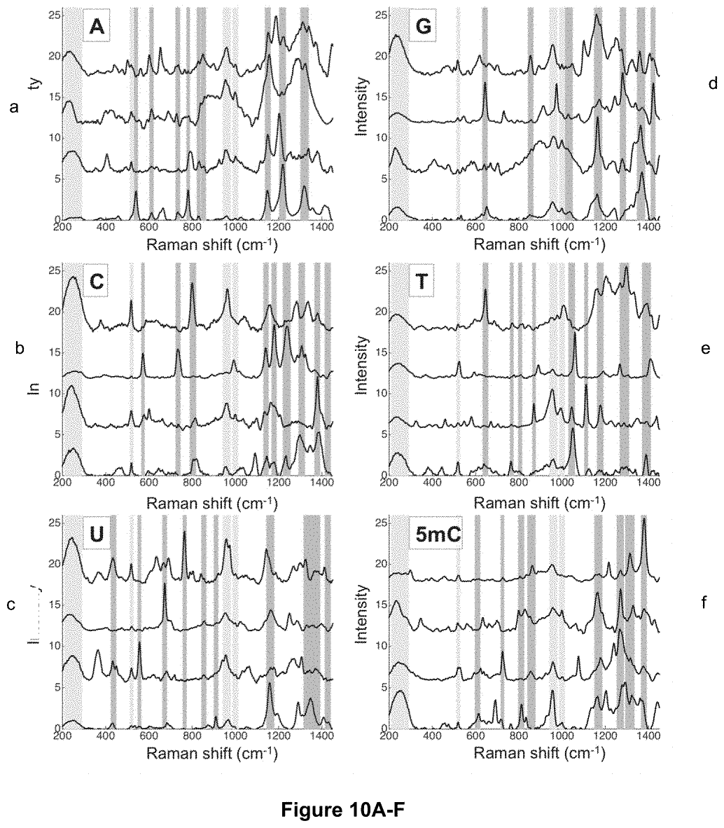

Next the present inventors sought to establish an optical fingerprint for each of the DNA and RNA nucleotides (adenine, A; guanine, G; cytosine, C; thymine, T; uracil, U; and 5-methylcytosine, 5 mC) using sets of specific Raman peaks, in order to perform sequence identification of unknown DNA and RNA oligomers. Previous work from our group showed that characteristic sets of peaks in Raman spectra of DNA homopolymers on silver nanopyramid arrays could be used to distinguish the different DNA bases with high accuracy. Specifically, the present inventors sought to extend this approach in order to identify DNA and RNA nucleotides and epigenetic modifications from SERS measurements on the nanopillar substrates. To this end, the present inventors first generated a spectral library by carrying out SERS measurements on dilute solutions of poly-(dN)ₓ and poly-(rN)ₓ homopolymers (N=A, G, C, T, 5 mC, or U), where the length of the oligomer x was 5-10 nucleotides. For each library experiment, the present inventors diluted the sample to 10 nM in water, deposited a ˜0.1 μL droplet onto the substrate, and allowed it to dry completely before collecting Raman spectra. Average spectra from the library collection are shown in FIG. 10. As with poly-dC DNA, the spectra could be divided into two categories—those showing only significant background peaks, and those with additional peaks that were not present in the control spectra. The present inventors again observed variation in the peaks present in different spectra, presumably owing to variations in the molecular conformations within the hotspots. In order to determine the characteristic peak positions and perform peak assignment, the present inventors removed spectra containing only background peaks and averaged the remaining spectra for each sample, then fit each peak present to a Gaussian distribution using the following equation:

$$I ( \tilde{v} ) = \frac{1}{σ \sqrt{2 π}} exp ( - {( \tilde{v} - μ )}^{2} / 2 {σ}^{2} )$$

where I is the intensity, \tilde{v} is the Raman shift (in cm⁻¹), μ is the mean and a is the standard deviation. From each Gaussian peak fit, the present inventors extracted the peak center position and full width at half maximum (FWHM), which were later used for classification of unknown spectra. The peak positions and FWHM values for the peaks of interest are shown in a table in FIG. 2a, and tentative peak assignments for those peaks that had been previously identified in the literature are listed in Table 1. Note that some peaks have shifted as compared to the corresponding peaks reported on other substrates, which may be due to substrate-specific interactions with the molecules.

After identifying the characteristic peaks present in the library spectra, the present inventors next adapted a molecular identification algorithm to identify unknown DNA and RNA nucleobases from their individual Raman spectra. The algorithm is based on a previously developed method of identifying DNA bases from SERS measurements, and is outlined using an example spectrum in FIG. 2. As a first step, for each target class (i.e., each distinct nucleobase) an optimized subset of the previously identified characteristic peaks was chosen in order to minimize the overlap between peaks used for different classes (FIG. 2b). For this step, any peaks for a given class that showed significant overlap with peaks from another class or did not appear consistently across spectra were removed from the optimal peak set. To make identifications on unknown measurements, each unknown spectrum was compared to the set of optimal peaks for each class. To calculate the estimated probability that an unknown spectrum belongs to class Y, the area of the spectrum within the FWHM region of each peak in class Y was integrated, then this integrated peak area was summed over all peaks in class Y and divided by the total number of peaks in that class (FIG. 2c). This process was repeated for each target class to give a list of average integrated peak area values, which serve as estimates of the probabilities that the unknown belongs to each class (FIG. 2d). The class with the highest estimated probability (or average integrated peak area value) was called as the most probable class (FIG. 2e).

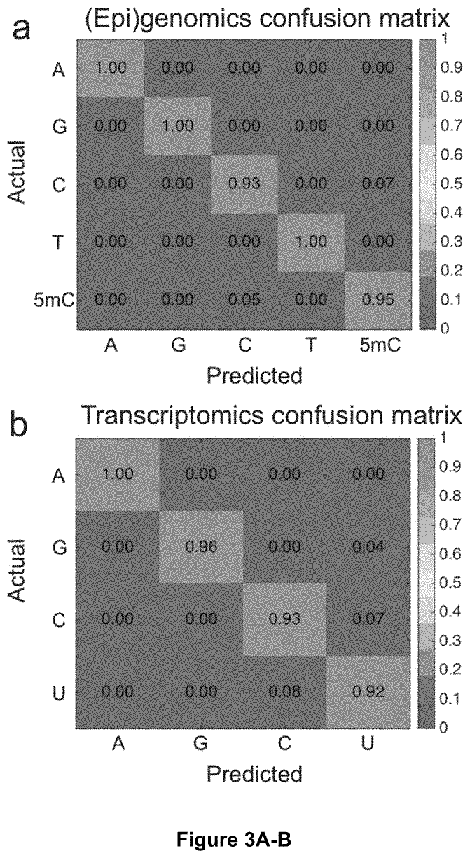

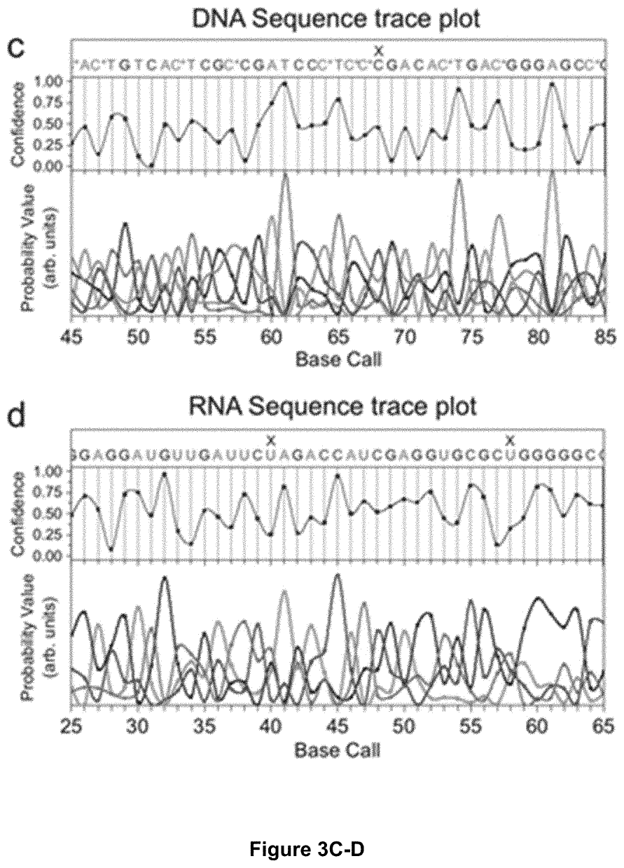

To assess the accuracy of the molecular identification algorithm, the present inventors applied the algorithm to discriminate between the DNA bases A, G, C, and T, as well as the epigenetic modification 5 mC, from a randomized library of Raman spectra collected on DNA homo-oligomers. Each ‘unknown’ spectrum was probabilistically classified as described above, and then the predicted class was compared to the actual class to generate a confusion matrix. The resulting (epi)genomics confusion matrix for DNA base calling is shown in FIG. 3a. As can be seen from the confusion matrix, the algorithm achieved a high accuracy for base calling among the five DNA bases, with an overall correct recall of 97.6%. In particular, the nucleobases cytosine and 5-methylcytosine were most likely to be confused with each other, which is not surprising given their structural similarity.

Next, the present inventors tested the viability of using the same molecular identification algorithm for discriminating between the four nucleobases present in RNA—A, G, C, and U—as would be necessary for single-molecule transcriptomics. Using the same approach of classifying each ‘unknown’ spectrum in a randomized library and comparing the predicted and actual classes, the present inventors generated a transcriptomics confusion matrix, as shown in FIG. 3b. As with DNA, base calling among homo-oligomers containing the four RNA bases was quite accurate, with an overall correct recall of 95.2%. Incorrect classifications for DNA and RNA bases were likely the result of a modest signal-to-noise ratio in many of the single-molecule spectra, which could erroneously increase the measured area within off-target peak regions. Previous SERS studies of small molecules on similar nanopillar substrates found a wide range of enhancement factors across different hotspots, which tended to average out over larger areas of the substrate. This issue could be mitigated in the future by filtering the data to include only spectra that originate from high-enhancement hotspots. Note that while these results were accomplished using single SERS measurements, it may also be possible to further increase the accuracy by collecting multiple measurements on each sample point, in analogy with increasing coverage in traditional sequencing methods.

### Example 3: Optical Sequence Identification of DNA and RNA

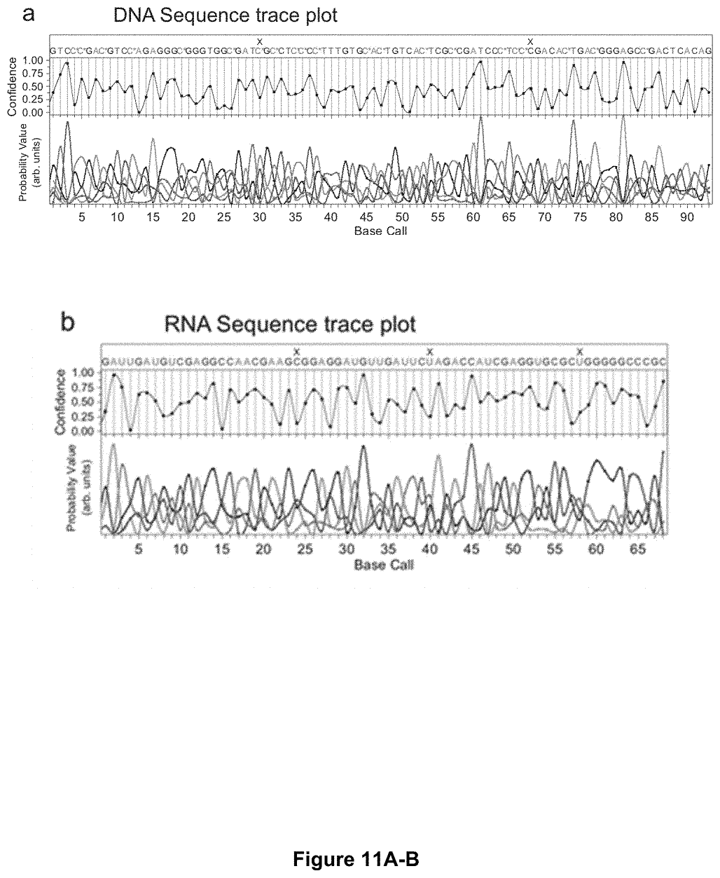

Next, the present inventors sought to test the invention's optical fingerprinting and molecular identification method in the context of single-molecule sequencing. To this end, the present inventors generated random ‘unknown’ sequences of DNA or RNA bases and pulled corresponding single measurements from our spectral library for each base. The measurements were then fed into the molecular identification algorithm to predict the sequence of the unknown, which the present inventors then compared to the actual generated sequence to produce a sequencing trace plot. Representative segments of resulting trace plots for DNA and RNA sequencing are shown in FIG. 3c, d, respectively (full trace plots shown in FIG. 11). In both cases, the algorithm was able to successfully predict the bases in the unknown sequence with a high degree of accuracy, with an error rate of <3% for DNA and <5% for RNA. The trace plots also display the calculated probability values for each of the possible nucleotides at each position of the unknown, as well as the resulting confidence, which is given by the normalized difference in probabilities between the first and second most probable classes. Although the accuracy is high, there is a large spread in the confidence of the base calls from one measurement to the next. This is again likely the result of a relatively low signal-to-noise ratio in some of the single-molecule measurements, which could be improved by using repeat measurements or filtering out very noisy data.

### Example 4: Raman Fingerprinting for Amino Acid Identification

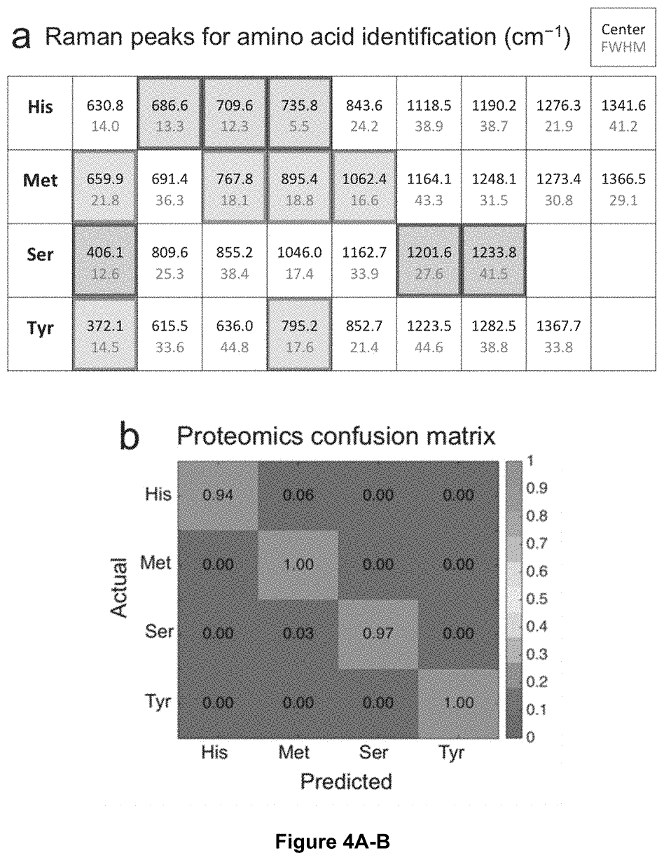

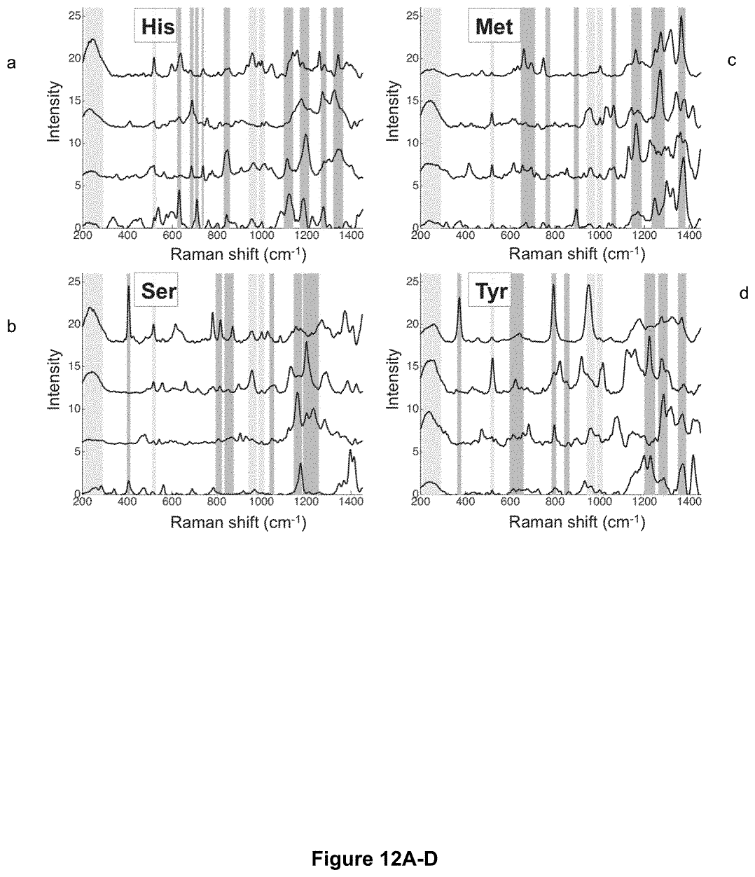

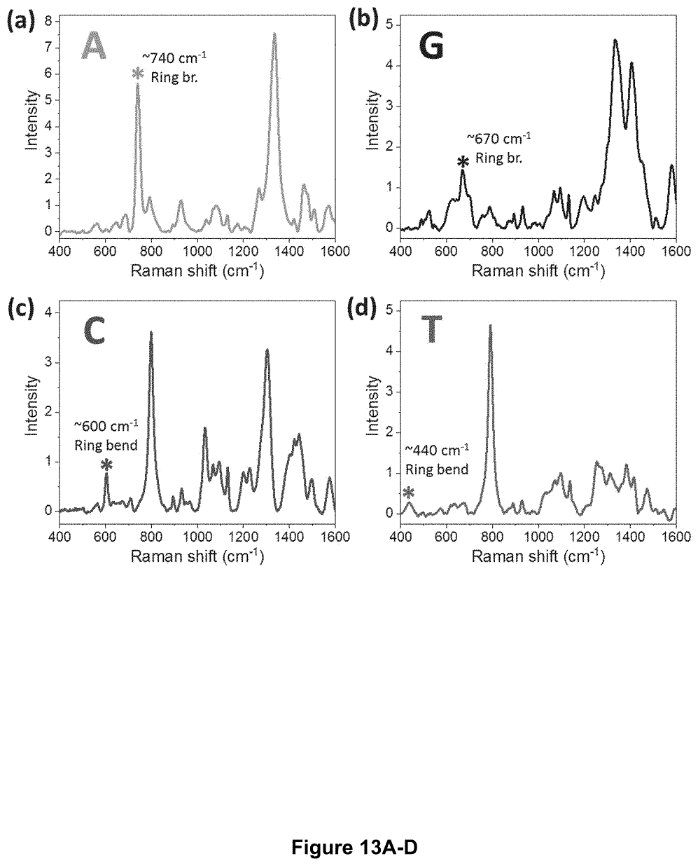

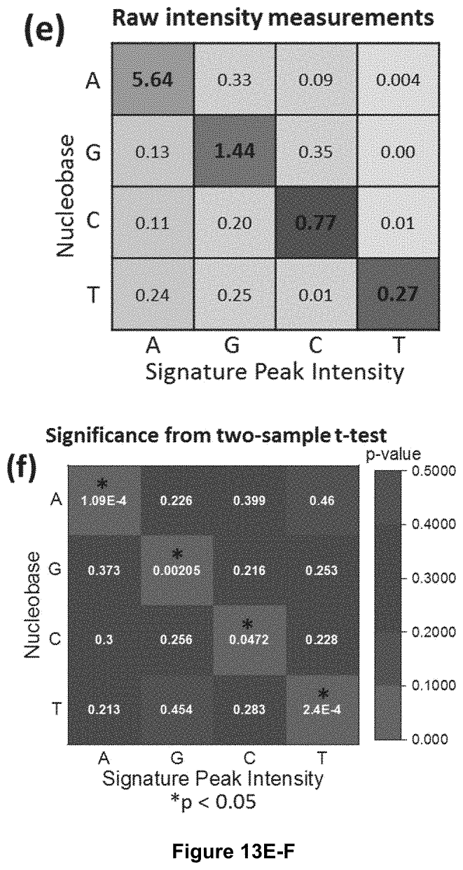

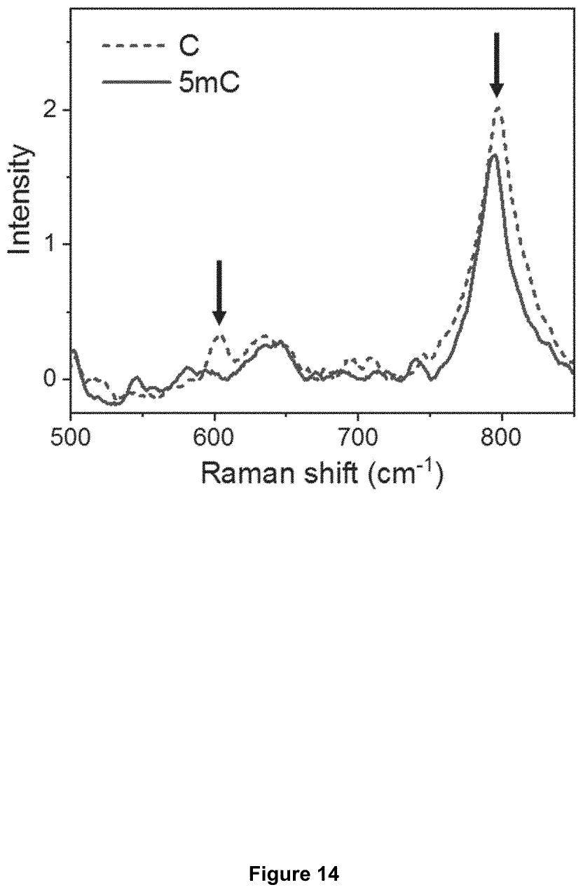

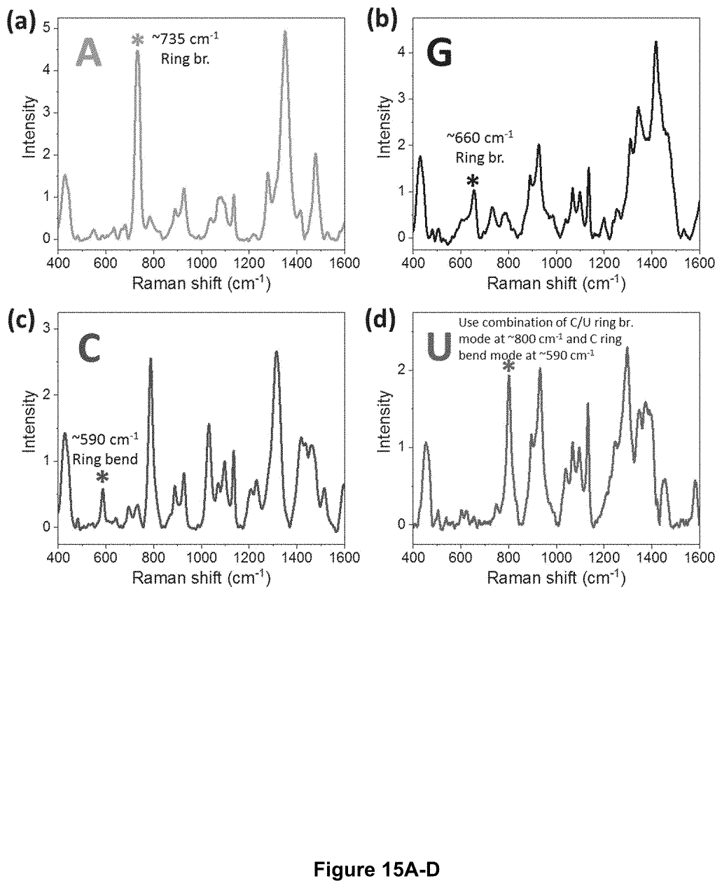

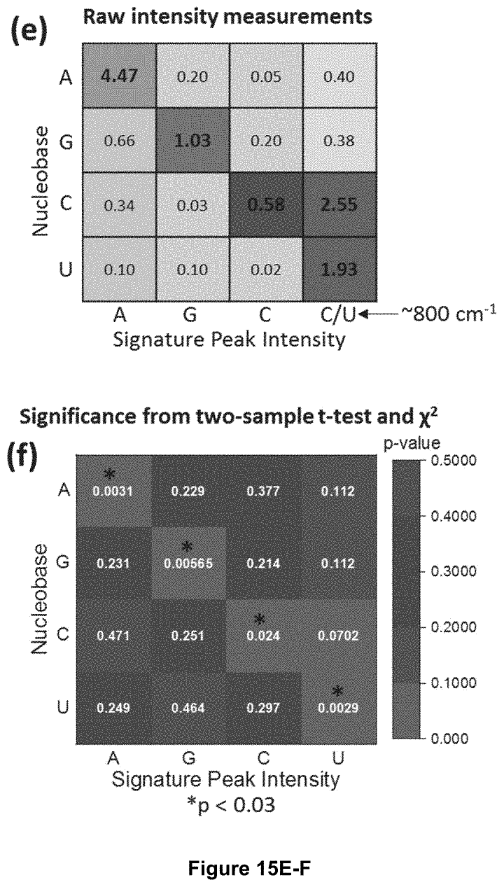

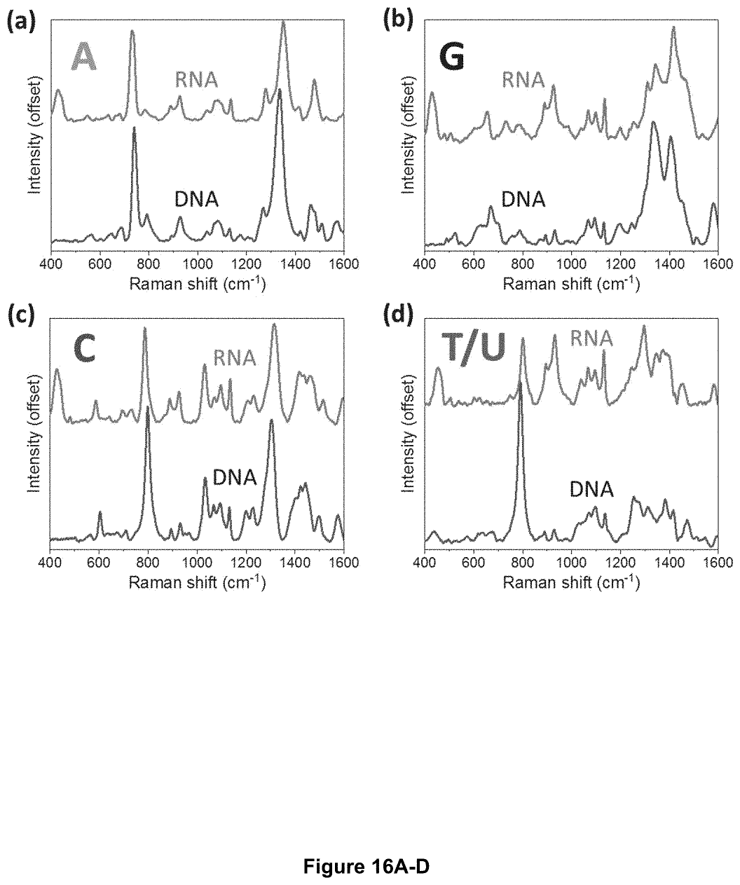

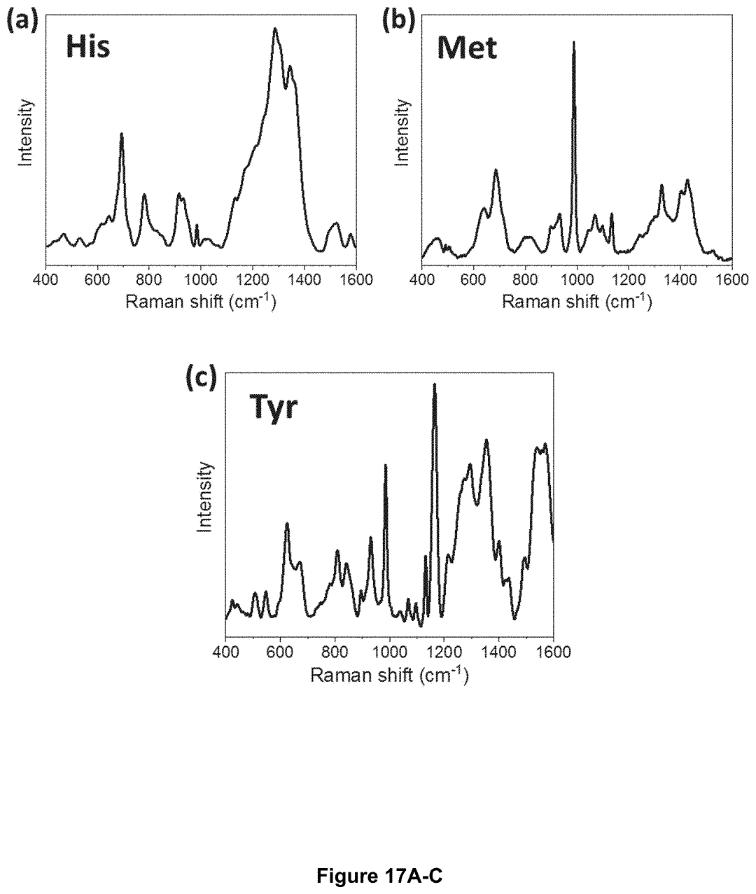

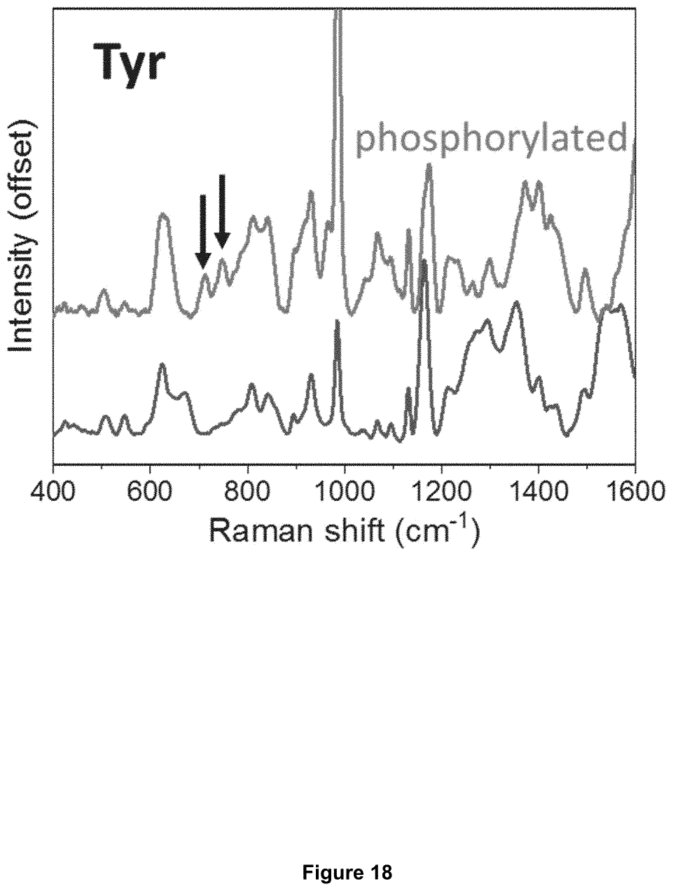

Finally, while the above work lays the foundation for single-molecule genomics and transcriptomics using SERS measurements, a similarly important challenge is to quickly identify individual protein molecules using optical measurements, which would enable translational profiling and proteomics at the level of single cells. Given the success in identifying nucleotides in single DNA and RNA molecules, the present inventors next sought to test whether this same approach could be extended to discriminate between different amino acids within peptides and proteins. The present inventors demonstrated discrimination between four different amino acids—histidine (His), methionine (Met), serine (Ser), and tyrosine (Tyr)—to enable the feasibility of using the optical sequencing approach for single-molecule proteomics. To do this, the present inventors adsorbed small quantities of four different poly-(X)₅ polypeptides (X=His, Met, Ser, Tyr) onto different areas of the nanopillar substrates from 0.1 uL solution droplets containing 10 nM polypeptide. Raman spectral grids were collected within each area and their spectra filtered to remove those showing only background peaks, forming the basis for the peptide library. The remaining library spectra were averaged, and Gaussian peak fitting was performed on each average spectrum, and the peak fitting parameters (peak center position and FWHM) were extracted to identify characteristic peaks for each amino acid (FIG. 4a). Average spectra and corresponding Gaussian peak fitting is shown in FIG. 12 for all four amino acids.

In order to test the invention's method for fingerprinting and identification of peptides, the present inventors next modified the molecular identification algorithm that was previously used for DNA/RNA base calling and applied it to differentiate between the four chosen amino acids. For this purpose, the present inventors again limited the chosen peaks for each molecule to an optimized subset of the characteristic peaks in order to improve classification and minimize overlap between the different peak sets. The present inventors then applied the algorithm to a randomized library of homopolypeptide spectra containing either His, Met, Ser, or Tyr, classified each ‘unknown’ spectrum as one of the four known classes, and compared the predicted classes to the actual classes to generate a confusion matrix. The results of this classification are shown in FIG. 4b. Discrimination between the four amino acids showed an overall accuracy of 97.7%, which is comparable to the accuracy observed for DNA and RNA base calling. This result highlights the generality of this approach for discriminating between chemically distinct monomers in biomolecules and suggests that SERS fingerprinting could potentially be useful for identifying single protein molecules based on relative amino acid content.

### Example 5: Optical Identification Material and Methods

Nanopillar Substrates:

All experiments were carried out using commercially available silver-coated leaning nanopillar ‘SERStrate’ substrates (Silmeco, Denmark). Substrates were received as ˜16 mm² squares and were stored under an inert atmosphere until use. Substrates were used as received and no prior cleaning step was performed.

RNA Handling:

Precautions were taken to minimize enzymatic degradation of the RNA. All solutions coming into contact with RNA were prepared with ultrapure deionized (DI) water (Barnstead Thermolyne NANOpure Diamond purification system, water resistivity >18 MΩ·cm). Prior to handling RNA, the workbench, gloves, pipets and other surfaces were cleaned with RNaseZAP™ RNase inhibitor solution (Ambion, Inc, USA). RNA solutions were stored long-term at −80° C. and short-term at −20° C. in small aliquots and were thawed on ice immediately before use.

Biomolecule Adsorption:

The DNA, RNA or peptide molecules were diluted to a concentration of 10 nM in ultrapure DI water (resistivity >18 MΩ·cm) and were adsorbed onto the substrate from a small droplet (˜0.1 μL). The droplet was then allowed to evaporate completely, during which time the surface tension at the air/liquid/solid interface of the receding droplet caused the pillars to lean into one another and trap some of the molecules in hotspots between the pillars.

Raman Spectroscopy:

Data was acquired using a Horiba LABRAM HR Evolution Raman Spectrometer. For each sample droplet area, several hundred Raman measurements were acquired pointwise along a grid within the droplet area, with a grid spacing of approximately 10 μm. Excitation was achieved using a 532 nm laser operating at 5% power with 0.5 s acquisition times. Scattered light was collected through a 100× microscope objective and passed through a 600 gr/mm grating before reaching the detector.

Data Analysis:

The disclosed algorithms, methods, techniques, and systems may be implemented in a digital computer system (1). Such a digital computer is well-known in the art and may include one or more of a central processing unit, one or more of memory and/or storage, one or more input devices, one or more output devices, one or more communications interfaces, and a data bus. In some embodiments, the memory may be RAM, ROM, hard disk, optical drives, removable drives, etc. In some embodiments, storage may also be included in the disclosed system. In some embodiments, storage may resemble memory that may be remotely integrated into the system. The input and output devices may be, for example one or more monitors, display units, video hardware, printers, speakers, lasers, spectrophotometers, filters, collectors, cameras, etc.

In accordance with any of the digital computer system (1) or computer(s) 1, these may be generally described as general purpose computers with elements that cooperate to achieve multiple functions normally associated with general purpose computers. For example, the hardware elements may include one or more central processing units (CPUs) for processing data. The computer 1 may further include one or more input devices (e.g., a mouse, a keyboard, etc.); and one or more output devices (e.g., a display device, a printer, etc.). The computers may also include one or more storage devices. By way of example, storage device(s) may be disk drives, optical storage devices, solid-state storage device such as a random access memory (“RAM”) and/or a read-only memory (“ROM”), which can be programmable, flash-updateable and/or the like.

Each of the computers and server described herein may include a computer-readable storage media reader; a communications peripheral (e.g., a modem, a network card (wireless or wired), an infra-red communication device, etc.); working memory, which may include RAM and ROM devices as described above. The server may also include a processing acceleration unit, which can include a DSP, a special-purpose processor and/or the like.

The computer-readable storage media reader can further be connected to a computer-readable storage medium, together (and, optionally, in combination with storage device(s)) comprehensively representing remote, local, fixed, and/or removable storage devices plus storage media for temporarily and/or more permanently containing computer-readable information. The computers and serve permit data to be exchanged with a network (2) and/or any other computer, server, or mobile device.

The computers and server also comprise various software elements and an operating system and/or other programmable code such as program code implementing a web service connector or components of a web service connector. It should be appreciated that alternate embodiments of a computer may have numerous variations from that described above. For example, customized hardware might also be used and/or particular elements might be implemented in hardware, software (including portable software, such as applets), or both. Further, connection to other computing devices such as network input/output devices may be employed.

It should also be appreciated that the method described herein may be performed by hardware components or may be embodied in sequences of machine-executable instructions, which may be used to cause a machine, such as a general-purpose or special-purpose processor or logic circuits programmed with the instructions to perform the methods. These machine-executable instructions may be stored on one or more machine readable mediums, such as CD-ROMs or other type of optical disks, ROMs, RAMs, EPROMs, EEPROMs, magnetic or optical cards, flash memory, or other types of machine-readable mediums suitable for storing electronic instructions. Alternatively, the methods may be performed by a combination of hardware and software.

The term “software” as used herein shall be broadly interpreted to include all information processed by a computer processor, a microcontroller, or processed by related computer executed programs communicating with the software. Software therefore includes computer programs, libraries, and related non-executable data, such as online documentation or digital media. Executable code makes up definable parts of the software and is embodied in machine language instructions readable by a corresponding data processor such as a central processing unit of the computer. The software may be written in any known programming language in which a selected programming language is translated to machine language by a compile, interpreter or assembler element of the associated computer.

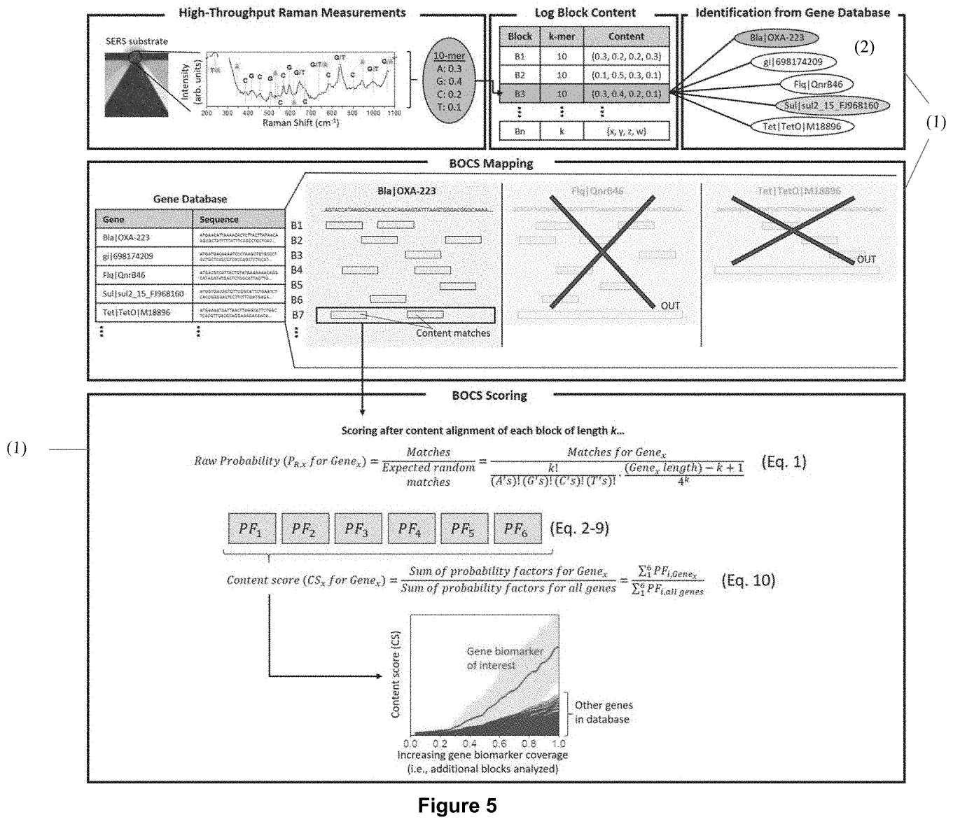

Considering the foregoing exemplary computer and communications network and elements described therein, In connection with one embodiment of the invention, it may be considered a software program or software platform with computer coded instructions that enable execution of the functionality associated with the systems and methods described generally in FIG. 5 and elsewhere. More specifically, the invention may be considered a software program or software platform that executes the BOCS algorithm based on data inputs to the algorithm as described including, without limitation, the DNA k-mer content data outputs generally described in FIG. 5 and elsewhere.

In connection with another embodiment of the invention, it may be considered a combined software and hardware system including (a) a software program or software platform with computer coded instructions that enable execution of the functionality associated with the digital computer system (1) along with the execution of the BOCS algorithm to generate block optical content, and (b) hardware elements including the hardware, such as optical hardware such as Surface-enhanced Raman spectroscopy (SERS) as generally described herein that may be used to analyze a SERS substrate.

### Example 6: The BOCS Algorithm

Given the capability of high-throughput single-molecule Raman spectroscopy measurements in determining DNA k-mer content, the need arises for a way to correlate these content measurements into meaningful genetic information. The potential for coupling a high-throughput measurement system with a broad-spectrum genetic biomarker identification method could lead to a diagnostic platform for rapid point-of-care genetic profiling. Direct applications range from providing clinicians with the information they need to effectively treat multidrug-resistant (MDR) bacterial infections to early detection of cancers and other genetic diseases that previously had no screening techniques. Therefore, the present inventors introduced the BOCS algorithm, which uses DNA k-mer content for broad-spectrum genetic biomarker recognition. In designing BOCS (schematic in FIG. 5), the present inventors took inspiration from probability-based sequence analyzers such as those employed for protein identification from mass spectrometry data, as well as alignment programs used to map next-generation sequencing reads to reference genomes.

In a similar nature to these methods, the BOCS algorithm relies on probabilistic content alignments to reference sequences for genetic biomarkers. The BOCS algorithm requires 1) the log of all k-mer blocks and their content and 2) a database containing gene sequences for the genetic biomarkers being investigated (e.g., antibiotic resistance, cancer, or other genetic diseases). The algorithm cycles through each k-mer block and performs a content-based alignment with each gene sequence in the database, translating through the gene sequence one nucleotide at a time and tracking the number of match locations—where the k-mer block content matches the content of the k-length gene sequence. A probability is calculated for each gene after each block is aligned with it. This raw probability (P_{R}) is simply the number of observed matches divided by the calculated number of matches that are statistically expected to occur randomly. It is based on the fundamental idea that genes in the database that are most similar to the k-mer blocks in terms of their content should have the most matches during alignment, and therefore deviate the most significantly from the random case. The raw probability is calculated from the number of match locations (m), the length of the k-mer block (k) and its content in terms of the number of A-G-CT nucleotides, and length of the gene (g_{L}), shown below for an arbitrary gene (x):

$${P}_{R , x} = \frac{m}{\frac{k !}{A ! G ! C ! T !} \frac{{g}_{L , x} - k +}{{4}^{k}} 1} \qquad ( 1 )$$

In the case where no matches are found for a gene, the gene is given a penalty score in place of the raw probability (adjustable parameter for the algorithm, normally in the range of 0.01-0.10). After the analysis of a block (i.e., when the block has been content aligned to each gene in the database), this raw probability is normalized by the maximum raw probability observed for all genes (P_{R} becomes P_{R}*). While this raw probability itself is not the score on which biomarker identifications are made, it is the basis for many of the six probability factors that make up the overall content score.

After the content alignment of a block has been completed for all genes, and the raw probabilities are calculated for each gene, six probability factors (PF) that make up the content score (CS) are calculated for each gene. These PF values are designed as pattern recognition elements for a customized machine learning enhancement to the algorithm. They were designed to account for repeated trends observed throughout comprehensive analyses of match patterns during content alignment. The first probability factor (PF₁) is the cumulative percent difference from average of the normalized raw probability (PDiff) multiplied by the normalized cumulative raw probability, shown below for an arbitrary gene (x) after an arbitrary block (bₙ) in terms of normalized raw probabilities:

$$\begin{matrix}{PDIff}_{x} = {\sum }_{1}^{{b}_{n}} \frac{( {P}_{R , x}^{*} - {P}_{R , all}^{*} )}{{P}_{R , all}^{*}} & ( 2 ) \\ {PF}_{1 , x} = {PDiff}_{x} \cdot \frac{{\sum }_{1}^{{b}_{n}} {p}_{R , x}^{*}}{{\sum }_{1}^{{b}_{n}} {p}_{R , all}^{*}} & ( 3 )\end{matrix}$$

The second probability factor (PF₂) is the total number of blocks, up to the current block, having at least one match from the content alignment:

PF_{2,x}=Σ₁^{b}^{ₙ}(P_{R,x}>penalty score) (4)

The third probability factor (PF₃) is the product of all normalized raw probabilities taken as the log base 2 sum. Since this leads to negative values, they are flipped by subtracting from the most negative value:

PF_{3,x}=max(|log₂P_{R,all}*|)−|log₂P_{R,x}*| (5)

The fourth probability factor (PF₄) is an exponential of the gene coverage (g_{cov}), indicating the fractional number of nucleotides within the gene that have been matched during content alignment:

PF_{4,x}=exp(500·g_{cov})/exp(500) (6)

The fifth probability factor (PF5) is the cumulative slope (SPF5) calculated from the percent difference from average of the normalized raw probability (PDiff, equation 2). The slope is calculated for the current block and the nine previous blocks; therefore, this factor does not take effect until the tenth block:

$$\begin{matrix}{S}_{PF 5 , x} = linear {fit ( \frac{{PDiff}_{x}}{max ( {PDiff}_{all} )} )}_{{b}_{n} - 9}^{{b}_{n}} & ( 7 ) \\ {PF}_{5 , x} = {\sum }_{1}^{{b}_{n}} {S}_{PF 5 , x} & ( 8 )\end{matrix}$$

The sixth probability factor (PF₆) is the cumulative difference from average of the normalized raw probability:

PF_{6,x}=Σ₁^{b}^{ₙ}(P_{R,x}*−P̅_̅{̅R̅,̅a̅l̅l̅}̅*̅) (9)

Each of the six PF values are normalized individually by the maximum PF observed for all genes (PF becomes PF*). This normalization by the maximum ensures equal weighting for the factors when they are added together to give the CS:

$${CS}_{x} = \frac{( {PF}_{1 , x}^{*} + {PF}_{2 , x}^{*} + {PF}_{3 , x}^{*} + {PF}_{4 , x}^{*} + {PF}_{5 , x}^{*} + {PF}_{6 , x}^{*} )}{{PF}_{1 , all}^{*} + {PF}_{2 , all}^{*} + {PF}_{3 , all}^{*} + {PF}_{4 , all}^{*} + {PF}_{5 , all}^{*} + {PF}_{6 , all}^{*}} \qquad ( 10 )$$

Notice that the CS is also normalized; however, here it is by the sum of CS values for all of the genes instead of the maximum as for the PFs. As each block is analyzed, the CS for each gene accumulates, leading to a probabilistic ranking of genes in the database. As demonstrated in the results, the compounded probabilistic content scoring is robust, and can often correlate the k-mer block contents to a positive genetic biomarker identification well below full coverage of the gene.

### Example 7: BOCS for Detection of Antibiotic Resistance

The BOCS algorithm may be built into a simulation for large-scale analyses. Such a simulation takes gene sequences from a biomarker database and creates k-mer blocks of A-G-C-T content to simulate BOS reads. These simulated BOS reads are then run through the BOCS algorithm against the biomarker database. The goal of the simulation is to see how well the BOCS algorithm can identify the correct gene (out of all others in the database) using merely randomized k-mer blocks of A G-C-T content. A specific gene from the database can be pulled or a random gene can be selected. The k-mer block lengths, gene coverage, and the number of errors within the blocks can all be set.

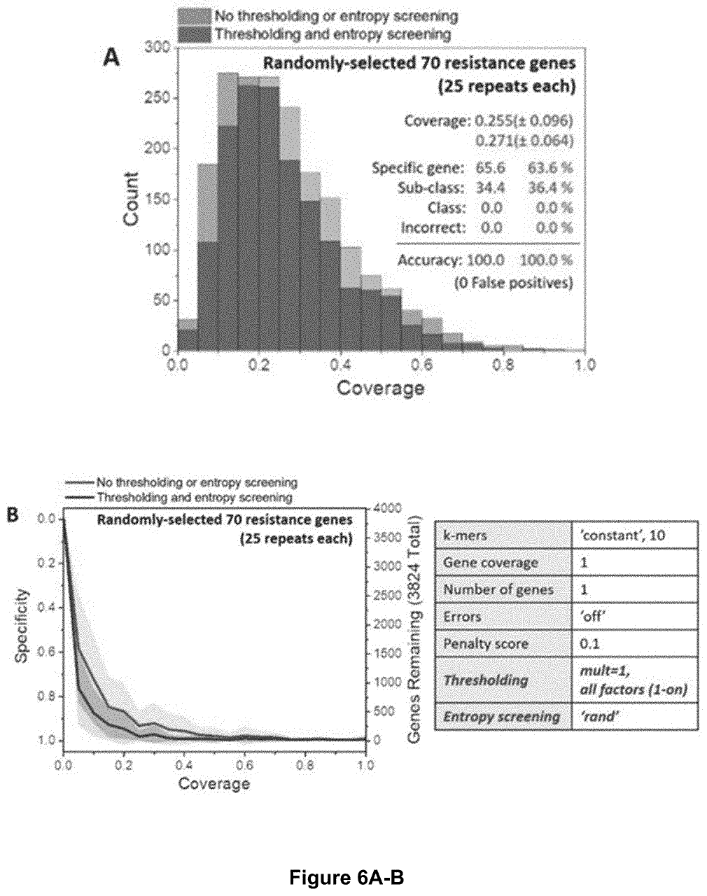

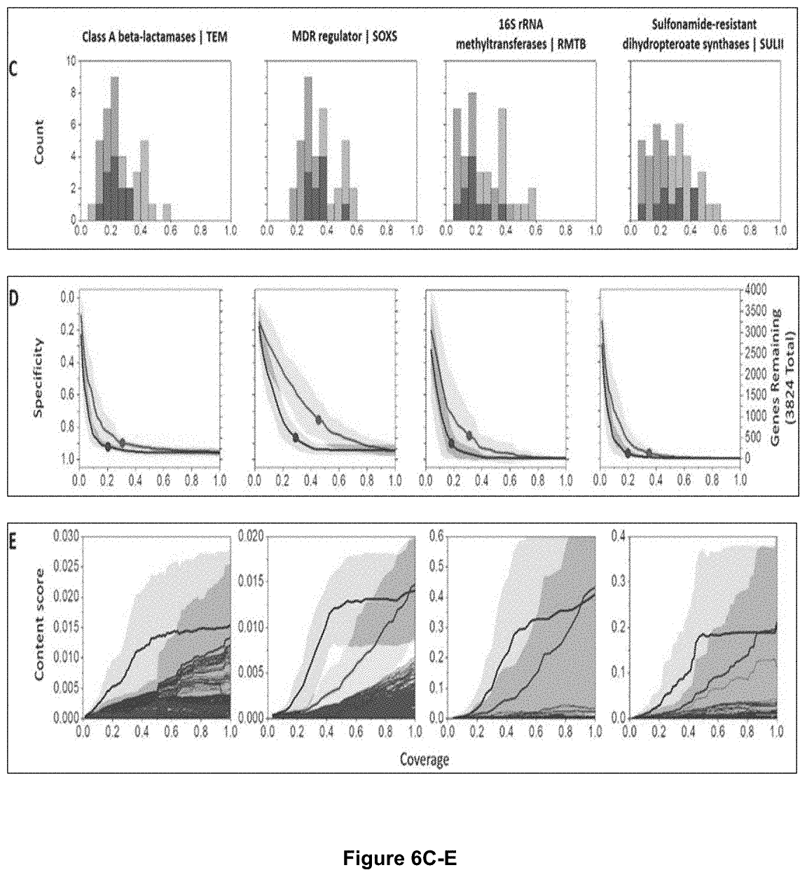

For comprehensive testing of the BOCS algorithm, the present inventors used the MEGARes database of antimicrobial resistance, composed of 3824 total resistance gene sequences. Due to the phylogeny of annotated genes in MEGARes and other gene databases, the BOCS analysis uses three levels for gene detection. In the order of most broad to most specific they include—class, sub-class, and specific gene. For example, a gene leading to resistance of tetracycline antibiotics could have a class: tetracycline ribosomal protection proteins, sub-class: TETO, and specific gene: TETO-x,y,z (where x, y, z are specific mutations of TETO). Note that deviations from the MEGARes three-level annotation system for more wide-range applicability with other genetic databases (as demonstrated later). For our BOCS benchmarking analyses, the present inventors randomly selected 70 genes having unique sub-classes from the MEGARes database (see the Supplementary Information Table S1 for details of the genes) and ran 25 repeat simulations on each, where each simulation repeat represents different split locations for the k-mer blocks and a different randomized order in which the blocks are analyzed. In this first set of 1750 simulations, the k mer blocks were set at k=10, single gene coverage, and no block errors (results are shown in FIG. 6).

In analyzing the simulation results, the present inventors were interested in four main metrics: accuracy, coverage at which a gene is identified, false positives, and specificity. The accuracy is a measure of how often the selected gene, which has been fragmented into randomized k-mers of A-G-C-T content, can be identified. The coverage at which a gene is identified indicates how many blocks less than the total (all blocks correspond to a coverage=1.0) are needed, eluding to the rapid, robust nature of the algorithm. False positives are a measure of the sensitivity in detection (more false positives means less sensitive). The specificity shows how significantly the gene database can be narrowed as consecutive blocks are analyzed. All of these factors depend on when an identification is made, which is determined as the point where a gene within the database adopts the highest content score and remains there and/or separates itself probabilistically from the rest. False positives arise when genes other than the selected gene meet this identification criterion. Genes within the database can be eliminated when a block shows no content matches during the alignment (this elimination scheme can only be used when there is single coverage for the genes and no block errors). In this first simulation with 70 resistance genes, 100% accuracy (with no false positives) was achieved while requiring an average coverage of merely 0.271±0.064 (FIG. 6A—red).

Additionally, roughly 90% of the genes in the MEGARes database were eliminated by 0.20 coverage (FIG. 6B—red). Results for four individual genes within the set of 70 are shown in FIG. 6C-E. Although variation in the coverage for identification and specificity are observed, both metrics remain highly favorable (identifications made and the majority of genes in the database eliminated at coverages <<1.0, FIG. 6C,D—red). FIG. 6E—red shows the rapid separation, and hence identification, of genes from the content scoring. In the case where content scoring separation does not appear as significant (such as for the TEM class A beta-lactamase), this is because all of the top-ranking genes (red line and gray lines in close proximity) are of the same TEM sub-class. Full results for this simulation can be found in Supplementary Table S2.

When looking at the content scoring for this first set of simulations on antibiotic resistance genes, the present inventors observed the most significant spikes in probabilities when the number of permutations for a particular block content was low (i.e., the value k!/(A! G! C! T!) was low). This led to the idea of preferably analyzing these ‘low entropy’ blocks before others in a process the present inventors call entropy screening. In the simulation, entropy screening can be applied in a random fashion (in the random order to which the blocks are scattered) or an ideal fashion (in order of low entropy to high entropy). Moreover, the present inventors noticed that in the majority of simulations, genes within the database that had probabilistically become irrelevant were still being analyzed as potential candidates. To alleviate this, the present inventors implemented a thresholding system to remove genes with lowest probability ranks after each round of block analyses. This type of thresholding based on content score ranking is also necessary to eliminate genes for the cases when there are more than a single gene or gene coverage as well as sequencing errors, where eliminations based on no content matches to a block would lead to significant identification error and decrease the overall accuracy. In the simulation, thresholding can be implemented based on the rank of the content score, as well as each of the individual probability factors, and each can be multiplied by a factor to increase/decrease the sensitivity of thresholding. With the thresholding and entropy screening in place, the first simulation with 70 resistance genes was re-run (again with k-mer blocks set at k=10, single gene coverage, and no block errors, with 25 repeat simulations per gene). Looking at the results shown in FIG. 6A,B—blue, the present inventors again saw 100% accuracy (with no false positives), this time achieved at an average coverage of only 0.255±0.096, and roughly 90% of the genes in the database were eliminated by 0.10 coverage. For four individual gene examples (FIG. 6C-E) significant improvements in BOCS metrics were seen for the case of thresholding and entropy screening. Not only did the present inventors achieve significant shifts towards lower coverage (FIG. 6C—blue) and higher specificity (FIG. 6D—blue), but it is seen faster, and with more prominent increases in the content scores for the genes the present inventors were attempting to identify (FIG. 6E—blue). Full results for this simulation can be found in Supplementary Information Table S3. This first round of simulations clearly demonstrated the rapidness to which BOCS can identify genes based merely on randomized k-mer content blocks, and improvements can be further seen with thresholding and entropy screening.

### Example 8: BOCS with Sequencing Variability

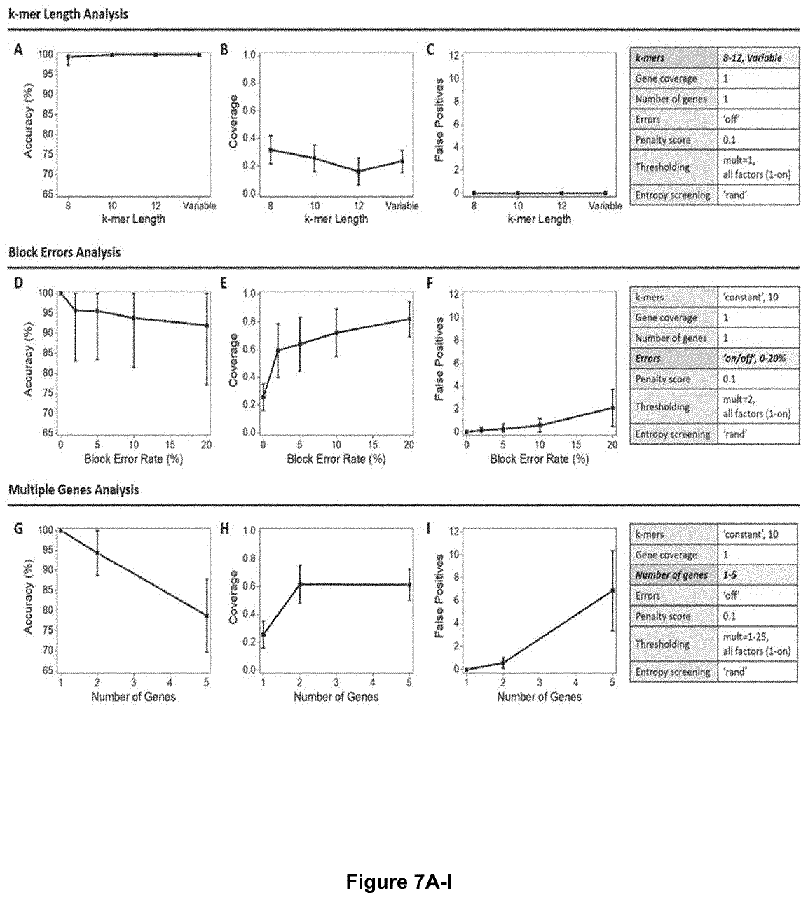

The present inventors next sought to test the limits of the BOCS algorithm by introducing sequencing variability in the form of fluctuating k-mer block lengths, block errors, and using blocks from multiple genes. All of these settings can be input on the BOCS simulation, and each of the simulations were run with the thresholding (using all probability factors and content score) and random entropy screening. First looking at k-mer lengths, the present inventors ran two sets of simulations with constant k-mer lengths different from the k=10 case used previously—one with k=8 and another with k=12. Then another set of simulations were run for varying k-mer lengths centered around k=10. For this, k-mer lengths for each block are randomly picked from a normal distribution centered around k=10, leading to a distribution of k-mer lengths in the range k=6-14. For each of these simulations, the same 70 MEGARes genes were used, again with 25 repeats. Results in FIG. 7A-C show that accuracy, coverage for identification, and the false positive rate are weakly correlated with the k-mer length variability. For all k-mer trials, the accuracy remains >99%, coverage for identification remains <0.40, and false positives remain <<1. Full results for these simulations can be found in Supplementary Information Tables S4-S6.

Next looking at block errors, a set of simulations (for the 70 resistance genes with 25 repeats) were run for each of four error rates within the blocks: 2, 5, 10, and 20%. Note that when using content as a sequencing platform, the error rates become double the rates that would normally be seen in single-letter sequencing. This is because a single point error within a k-mer block affects the resulting content of two nucleotides—the letter corresponding to the correct nucleotide, and the letter corresponding to the incorrect nucleotide. In the BOCS simulation, the error rates are entered as fractional error rates for the gene sequence, not the content; therefore, the error rates shown here (2, 5, 10, and 20%) were entered as 0.01, 0.025, 0.05, and 0.10. The results in FIG. 7DF indicate that accuracy, coverage for identification, and false positive rate are more strongly correlated to block errors than is the k-mer length, although all of these metrics remain strong even under extreme error rates. At error rates as high as 20%, the average accuracy remains >90%, the coverage for identification never reaches 1.0, and false positives are low (under 2 false positives on average). Full results for these simulations can be found in Supplementary Tables S7-S10.

Lastly looking at using k-mer blocks from multiple genes instead of a single gene (and therefore trying to identify all genes from which the blocks are compiled), the present inventors ran two sets of simulations using sets of k-mer blocks from two and five genes. The 2-gene simulations are for 10 random 2-gene selections from the base set of 70 resistance genes, each with 25 repeats. The 5-gene simulations are for 5 random 5-gene selections from the base set of 70 resistance genes, each with 25 repeats. FIG. 7G-I shows accuracy decreases linearly with an increasing number of genes, but remains near 80% for five genes, with average coverage around 0.60. The main hindrance with an increasing number of genes is the large false positive rate, which reaches an average of >6 when the blocks are comprised of five genes. This makes sense when thinking about the relative signal from each gene—when the k-mer blocks are comprised of five different genes, the signal-to-noise level can be as low as 1:4 for each of the genes. The fact that an 80% accuracy rate is observed despite this low signal-to-noise level is impressive, and in the future, more advanced machine learning techniques could be applied to the BOCS algorithm to help reduce the false positive rate. Full results for these simulations can be found in Supplementary Tables S11-S12. In all, the BOCS algorithm proved very robust under the pressures of variable k-mer lengths, high block error rates, and in the presence of blocks comprised of multiple genes.

### Example 9: BOCS for Determining Clinical MDR Bacterial Strains

The present inventors applied BOCS simulations towards the detection of a very relevant clinical MDR bacterial strain. Methicillin-resistant Staphylococcus aureus (MRSA) has become a leading cause of bacterial infections in healthcare and the community. It is the most clinically-relevant Staphylococcus species, with a large prevalence of tissue and bloodstream infections due to chronic skin conditions and surgical procedures. Through horizontal gene transfer, MRSA strains show resistance to most beta-lactam antibiotics, leading to endemics in healthcare facilities worldwide. Diagnosis is most commonly performed with phenotypic cell culture assays. These assays look for the presence of the mecA gene encoding the PBP2a penicillin-binding protein with a cefoxitin (a beta-lactam, with resistance being of the type OXA class D) antibiotic inducer. The culture tests must incubate for >24 hours, with overall time for testing usually being >46 hours.

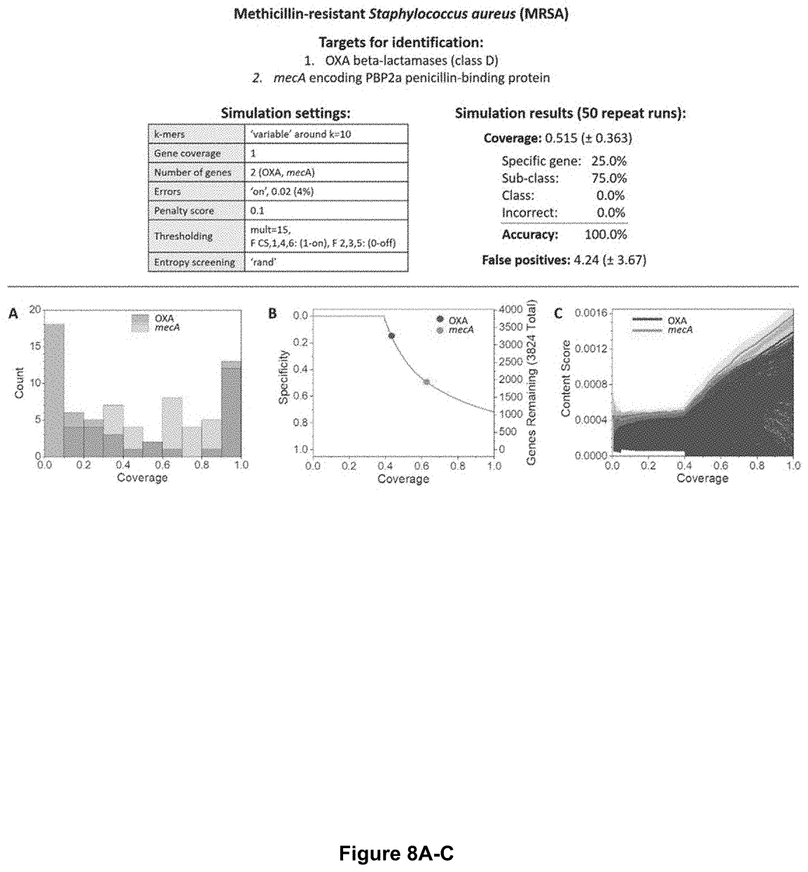

To demonstrate detection of MRSA with BOCS, the present inventors designed a simulation looking for two genes: 1) mecA gene encoding the PBP2a penicillin-binding protein and 2) OXA beta lactamase (class D). The simulation used variable length k-mer blocks centered around k=10 (for a range of k=6-14), and a 4% error rate within the blocks. Thresholding (with multiplier and selected factors) and random entropy screening were also applied, and the simulation was run with 25 repeats. The BOCS algorithm once again showed powerful performance in identification of the two resistance genes of interest, leading to MRSA detection even in the presence of block errors and variable k-mer lengths (results in FIG. 8). Accuracy was 100%, with identification being made at an average coverage of 0.416±0.296. The false positive rate was low (0.520±0.510), and most of the sparse false positives were genes conferring beta-lactam resistance or general MDR effluxes. FIG. 8A shows a histogram of the coverage for identification of both the mecA and OXA genes throughout all 25 repeats, and FIG. 8B shows the specificity as coverage increased. FIG. 8C shows increasing content score with coverage, clearly illustrating how the mecA and OXA genes of interest probabilistically separate themselves from the rest of the genes in the database, leading to their identification at low coverages. This MDR detection simulation further demonstrates the robustness of the BOCS algorithm and its potential for clinical diagnostics.

### Example 10: Applying BOCS to Cancer and Other Genetic Disease Databases

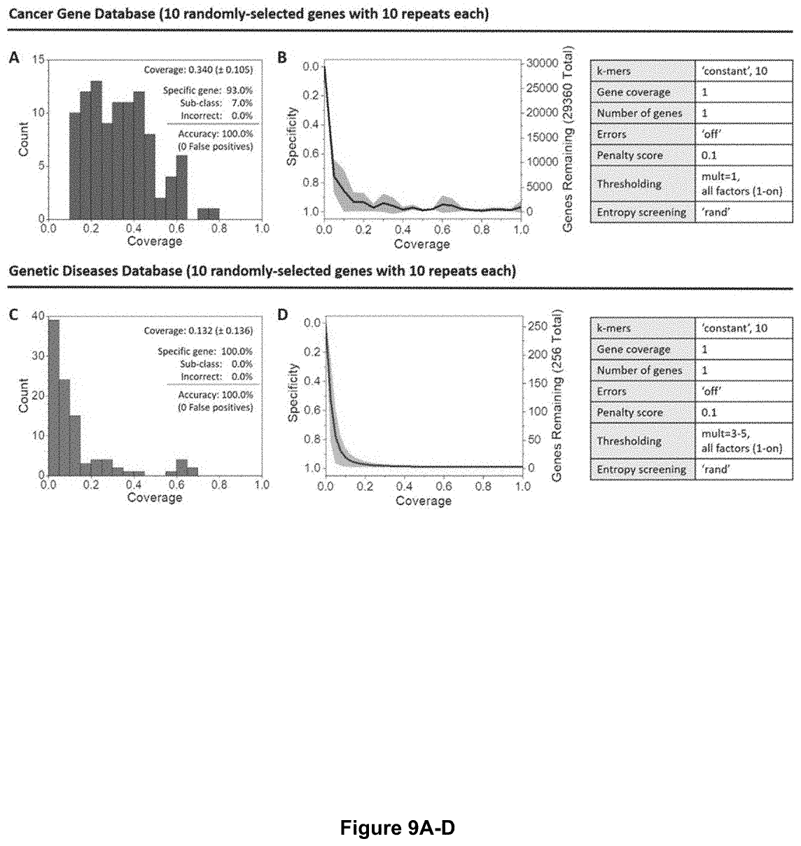

Expanding BOCS to other areas benefiting from broad-spectrum diagnostics, the present inventors ran simulations with the COSMIC cancer database and a custom compiled database of other genetic diseases including many listed by the NIH Undiagnosed Diseases Network. Note for these databases, there is no class level identification, only sub-class and specific gene. For each database, 10 randomly-selected genes were run with 10 repeats, for 100 total simulations with constant k-mers at k=10, no block errors, and thresholding and entropy screening (results in FIG. 9). Cancer genes (FIG. 9A,B) showed 100% accuracy (no false positives) at an average coverage for identification of 0.340±0.105 and specificity on par with that of the resistance genes. The other genetic diseases (FIG. 9C,D) showed 100% accuracy (no false positives) with an average coverage and specificity significantly better than the resistance genes. The average coverage for identification was 0.132±0.136, and roughly 95% of the genes within the database were eliminated by 0.10 coverage. Full results for these simulations can be found in Supplementary Information Tables S13-S16. The fact that other genetic biomarker databases perform as well or superior to our results with the resistance database adds to the vast potential of the BOCS algorithm in its ability for broad-spectrum diagnostics.

### Example 11: Optical Sequencing Measurements with BOCS Algorithm for the Characterization of a β-Lactamase Gene within the Pathogen of Origin

In one embodiment, the present inventors successfully coupled optical sequencing measurements with the content-scoring algorithm, or BOCS algorithm for the characterization of a β-lactamase gene within the pathogen of origin. Specifically, we show that merely a few highly accurate measurements of DNA k-mer block content (<<full coverage of the gene) from silver nanoparticles can be used with the content-scoring algorithm to identify the correct OXA β-lactamase (class D) gene from a comprehensive antibiotic resistance database and confirm the Pseudomonas aeruginosa pathogen from which it originates. Although optical sequencing measurements can be multiplexed using silver-coated nanopyramid substrates for SERS, we utilized metallic nanoparticles here to demonstrate broader applicability across plasmonic substrates and varying resolution (single molecule versus ensemble). We also show extensions to transcriptomics and epigenomics. Ultimately, the results here demonstrate the use of an optical sequencing platform as a diagnostic for inexpensive and rapid identification of broadspectrum genetic, transcriptomic, and epigenomic biomarkers.

### Example 12: Optical Sequencing Measurements with Positively Charged Silver Nanoparticles

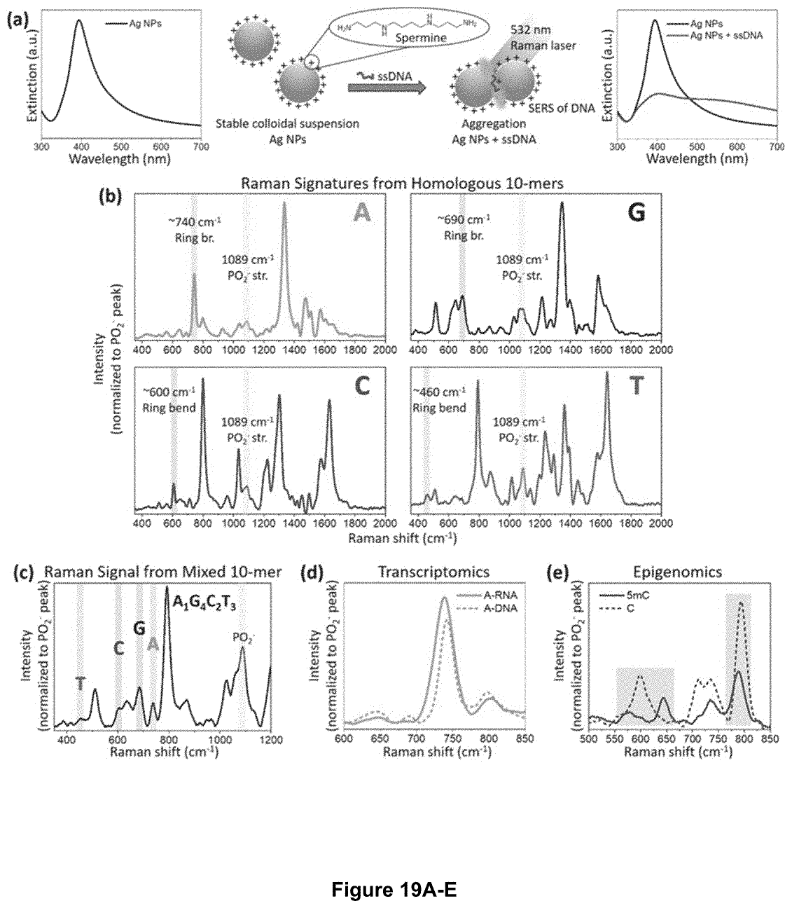

In this study, we collected optical sequencing measurements from ssDNA k-mer blocks with positively charged, spermine-coated silver nanoparticles (Ag NPs) as the plasmonic substrate (FIG. 1a). Recent work has shown strong, reproducible SERS signal from a range of substrates like single DNA molecules on nanopyramid substrates and ensemble measurements from ˜25 nm cationic nanoparticles for ssDNA, dsDNA, and RNA. The Ag NPs remain stable in the colloidal solution due to electrostatic repulsion of the positively charged ligands and show no significant background Raman signal. SERS signal is only achieved upon aggregation with the addition of negatively charged nucleic acids, which is DNA k-mer blocks for our measurements. As seen from the extinction spectrum in FIG. 19a, a strong localized surface plasmon resonance (LSPR) peak is observed at ˜392 nm for blank Ag NPs, and a large red shift is observed with the addition of DNA due to aggregation. When added to the Ag NPs, the DNA strands attach electrostatically to the nanoparticle surfaces and at interparticle hot spots leading to strong SERS excitation with a 532 nm Raman laser. In the analysis of all SERS measurements shown here, the present inventors perform a consistent signal processing (cosmic ray removal, smoothing, shift correction) and normalization (baseline subtraction, normalization to a standard peak). For sequencing applications, it is essential to first know the specific Raman signal, or signature, for each of the four nucleobases A, G, C, and T. To get these signatures, the present inventors performed SERS measurements on homologous 10-mer DNA sequences (i.e., poly(N)10, where N is A, G, C, and T). Shown in FIG. 19b, there exists a complex pattern of Raman peak features for each nucleobase over the range of 350-2000 cm-1 shift. For optical sequencing, we identified a single distinctive Raman peak for each nucleobase as the “signature peak”, which is later used to determine the content in unknown sequence blocks. For purines, we selected the ring breathing modes at ˜740 cm⁻¹ for A and ˜690 cm⁻¹ for G. For pyrimidines, we selected the ring bending modes at ˜600 cm⁻¹ for C and ˜460 cm⁻¹ for T.

Within each SERS measurement, the PO2⁻ stretching mode peak at 1089 cm⁻¹ due to the phosphate backbone is used as an internal standard for normalizing the relative peak intensities, as is consistent with other studies employing nanoparticle substrates. All signature peaks and the PO2⁻ normalization peak are highlighted in FIG. 19b. In the SERS signal from a mixed sequence DNA block, the four signature peaks are present with relative intensities (normalized to the PO2—peak) corresponding to their respective content, shown for a 10-mer DNA block with content A:1, G:4, C:2, and T:3 in FIG. 19c. These relative intensities in signature peak locations can, therefore, be used to deconvolute the signal from an unknown mixed sequence.

It is important to note that impactful extensions exist for transcriptomics and epigenomics by applying optical detection to RNA and chemically modified nucleobases. As shown in FIG. 19d for a homologous RNA sequence of repeating adenine, A, the signature peaks are nearly identical between RNA and DNA. This is evident by the similar ˜740 cm⁻¹ ring breathing mode peak for A. Additionally, small perturbations to the Raman spectrum can be seen due to modified nucleobases like the modification of cytosine, C, to 5-methylcytosine, 5 mC (also measured from homologous sequences), which plays an important role in gene regulation. Seen in the highlighted regions of FIG. 19e, the present inventors observed shifts and intensity changes to the signature peak of C at ˜600 cm⁻¹ and the strong ring breathing mode for pyrimidines at ˜800 cm⁻¹, which are consistent with previous studies. This opens the opportunity to directly apply our diagnostic optical sequencing platform to transcriptomic, RNA structure, and epigenomic studies.

### Example 13: Calibrating with DNA Block Standards

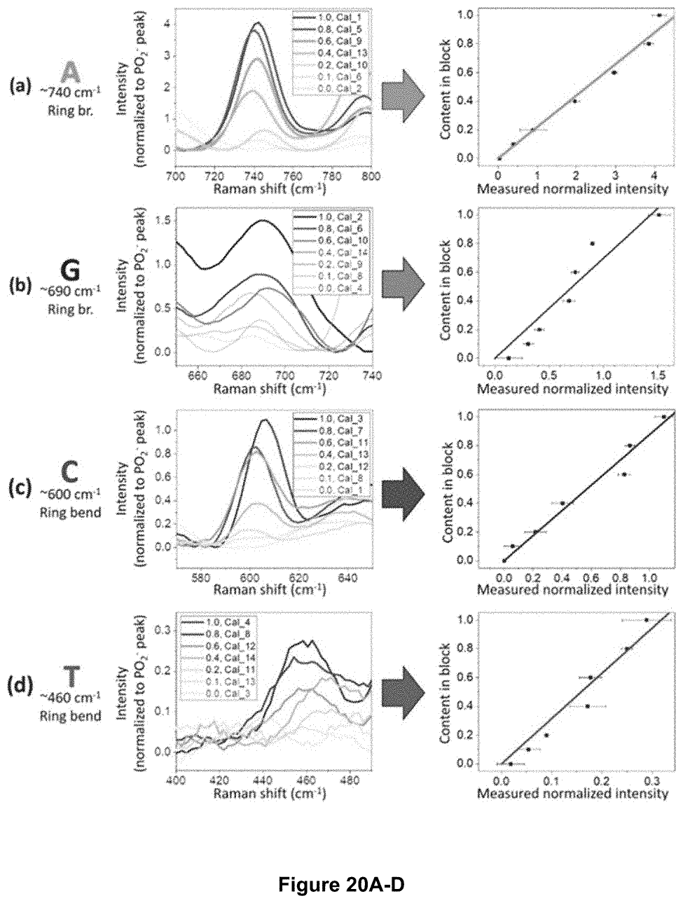

To fully deconvolute the A-G-C-T content of an unknown mixed sequence DNA k-mer block for optical sequencing, it may be necessary to know the full range of intensity values for signature peaks of each nucleobase. Therefore, we used custom DNA k-mer blocks with a known content as standards for generating content calibrations. The 14 calibration blocks are provided in Table 5. These 14 ssDNA 10-mer calibration blocks span the range of 0-1 fractional content for each of the four nucleobases. Blocks Cal_1, Cal_2, Cal_3, and Cal_4 provided the Raman signatures shown in FIG. 19b, as they are of content=1. Together, SERS measurements on the set of 14 calibration blocks were used to generate the calibrations shown in FIG. 20. On the left of FIG. 20, SERS spectra are plotted for increasing fractional content of a particular nucleobase (lighter to darker shades) in the zoomed-in region of the signature peaks. A direct, linear correlation is observed between the fractional content and the signature peak normalized intensity for each of the nucleobases, seen in the fitted data points on the right of FIG. 20 (data points and variance are from five technical replicates of each calibration block). The trends are all linear although the range of measured intensity values can vary significantly. Intensities for the adenine, A, signature peak at ˜740 cm⁻¹ range from 0 to >4, while the intensities for the thymine, T, signature peak at ˜460 cm⁻¹ range from 0 to 0.3. Linear fits to these trends, with intercept locked at zero, provide the finalized correlations, which can be used to determine the content in unknown DNA k-mer blocks.

### Example 14: Content Identification within Gene Blocks

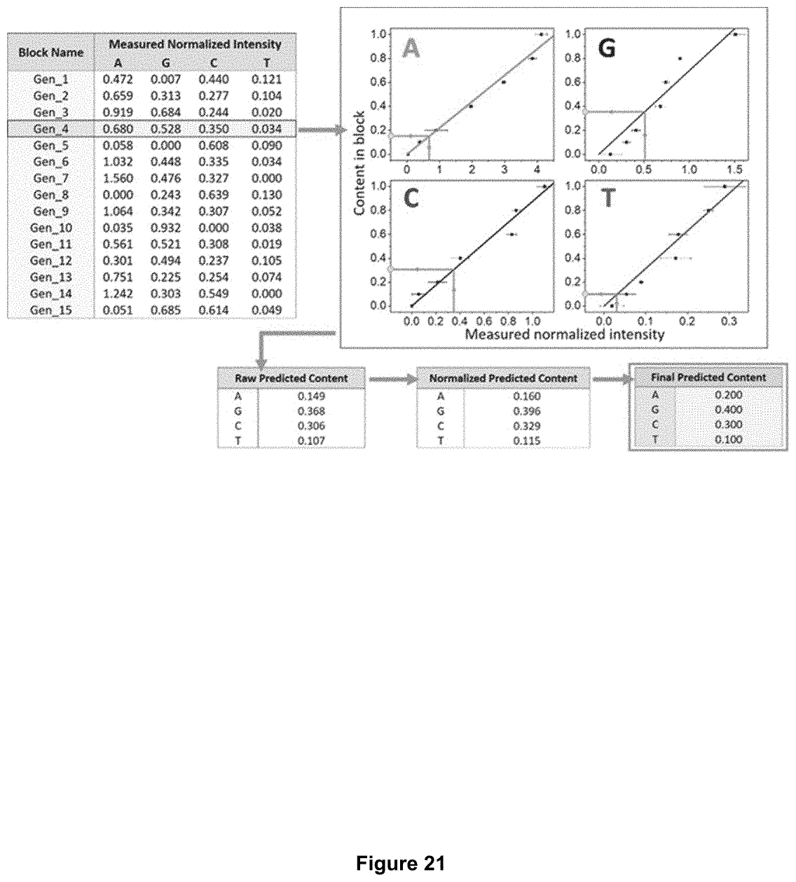

The present inventors applied the calibrations toward identifying content within k-mer blocks from an actual gene sequence, for subsequent integration with the content-scoring algorithm. The 15 gene blocks are provided in Table 6. These 15 ssDNA 10-mer gene blocks are from an OXA β-lactamase (class D) gene found in P. aeruginosa. Although 10-mers were used throughout this study, SERS measurements can be collected from longer blocks. From SERS measurements on the 15 gene blocks, the present inventors measured the normalized intensity for signature peaks (averaged from three technical replicates). FIG. 21 details the process of using these measurements for predicting content within the blocks. Using the Gen_4 block as an example, the measured normalized intensity for each of the four signature peaks is used to predict a content for each nucleobase A, G, C, and T by multiplying the measured intensity by the slope of the linear calibrations. This initial raw predicted content is then normalized such that the sum is equal to one. Knowing that each nucleobase must be present in integer quantities, the final predicted content can be determined via rounding. The final predicted content for the Gen_4 block agrees with the actual content given in Table 6.

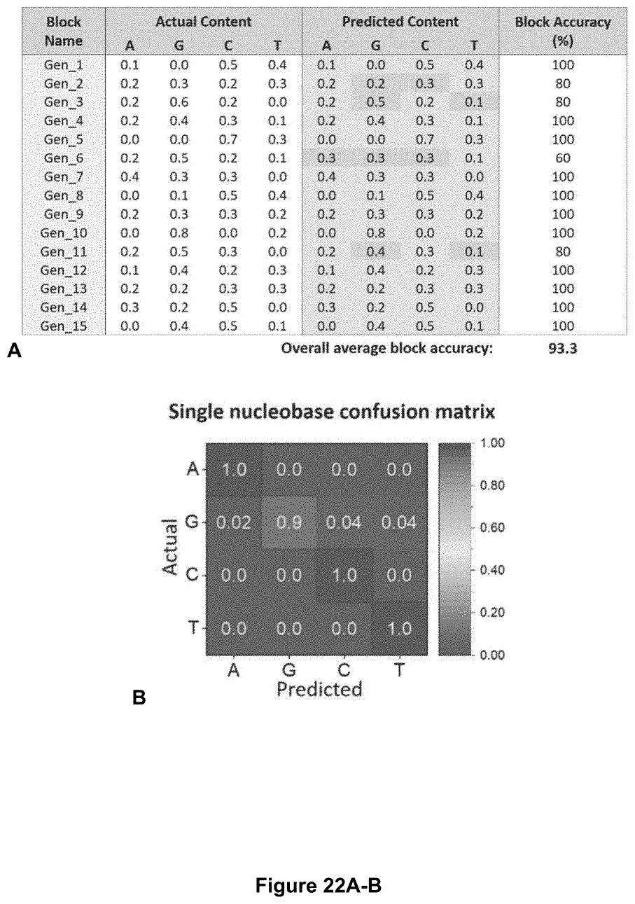

Predicted content for all 15 gene blocks is provided in FIG. 22, where the predictions are compared to the actual content. It is important to note that the accuracies reported in optical sequencing are different than those from traditional single-letter sequencing. Since reads are of A-G-C-T content and not letter-by-letter sequences, one misidentification results in a double error. This is because the content of the incorrect nucleobase and substituted nucleobase are both affected. A confusion matrix analysis on the single nucleobase level throughout all 15 blocks shows that the majority of errors result from guanine, G, content being under identified (˜10% of G bases throughout the gene blocks). In total, errors in the predicted content were present in only four of the blocks, three which resulted from a single nucleobase swap (80% accuracy for the block) and one which double-swapped nucleobases (60% accuracy for the block). The content in the other 11 blocks was predicted with 100% accuracy. Overall, the content was predicted at an average accuracy of 93.3%.

### Example 15: Multidrug-Resistant (MDR) Pathogen Profiling with Optical Sequencing

For full integration into a diagnostic method, the high-accuracy optical sequencing reads were coupled with the content-scoring algorithm for genetic biomarker detection. With the optical sequencing platform, we set out to demonstrate the detection of a P. aeruginosa infection with the drug-resistant β-lactamase gene. P. aeruginosa is a clinical multidrug-resistant (MDR) pathogen of critical importance due to its prevalence for causing bloodstream, urinary, and pulmonary infections in hospital settings, especially for immunocompromised patients in intensive care settings. Due to the multiple mechanisms of inherent and acquired resistance of this organism, patients infected with P. aeruginosa have limited therapeutic options. It is, therefore, imperative to have more early-stage, rapid diagnostic techniques in place to screen for P. aeruginosa so that effective antibiotic regimens can be prescribed from the onset of infection.

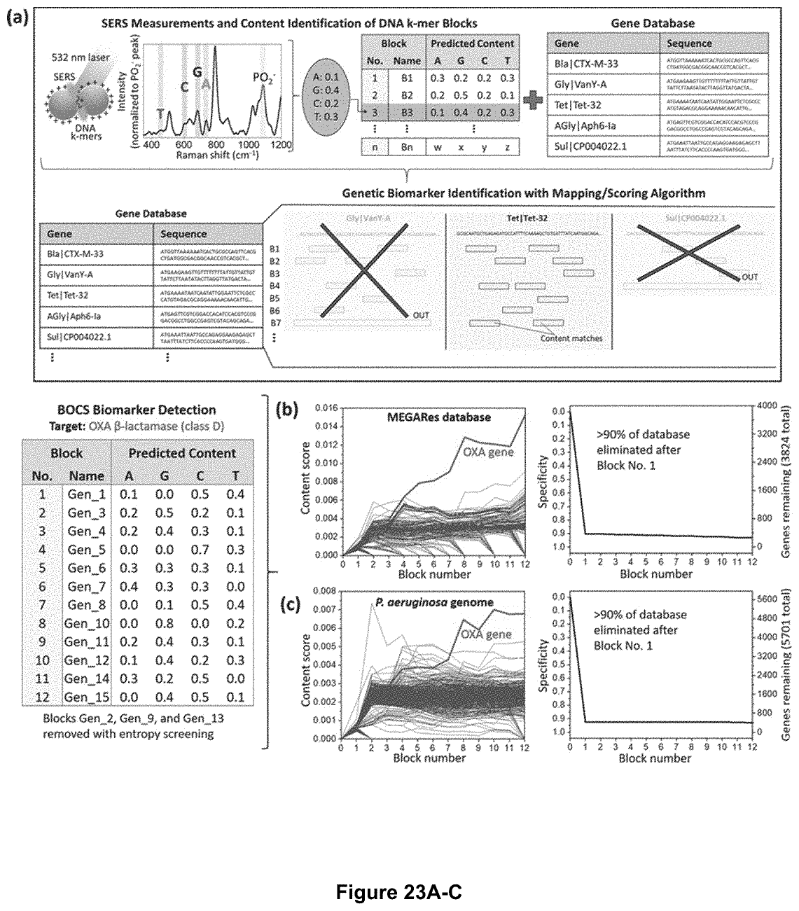

The content-scoring BOCS algorithm was developed to perform genetic biomarker database searching from measurements of the nucleotide sequence content. It operates analogously to probability-based sequence analyzers such as those employed for peptide identification from mass spectrometry data and alignment programs used for mapping next-generation sequencing reads to reference genomes. In a similar fashion, the algorithm relies on probabilistic content alignments to database sequences of genetic biomarkers. Outlined in FIG. 23a, the algorithm cycles through reads of the nucleotide sequence content (i.e., logged k-mer block content reads from optical sequencing) and performs a content-based alignment with each gene sequence in a database, translating through the gene sequence one nucleotide at a time. The alignment tracks the number of match locations, where the kmer block content matches the content of the k-length gene segment. The number of match locations is the fundamental parameter in a set of six probability factors that act as machine learning elements in the calculation of an overall content score. Genes in the database are probabilistically ranked, and identified, based on the content score as it compounds with more blocks being analyzed. The algorithm also incorporates logic elements such as a penalty score given to genes in the database where no matches are found during alignment, thresholding to eliminate low-ranking genes in the database that may skew the content-scoring, and entropy screening to eliminate reads that have a maximal number of permutations based on the content.

Thorough simulations of the BOCS algorithm with antibiotic resistance, cancer, and other genetic disease databases proved very robust, even under the pressures of variable k-mer block lengths, high error rates, and in the presence of blocks comprised of multiple genes. The present inventors ran the measured gene blocks with predicted content at 93.3% accuracy through the content-scoring algorithm against the MEGARes antibiotic resistance database comprised of ˜4000 known resistance genes, including the OXA β-lactamase (class D) gene of our measured gene blocks. This analysis demonstrates the ability of optical sequencing to diagnose antibiotic resistances from unknown samples with no prior knowledge of the pathogen or strain. The table of gene blocks and their predicted content, which was provided to the algorithm, is shown in the lower portion of FIG. 23. Note that three of the blocks were eliminated with the entropy screening functionality because these blocks had predicted content with a maximum number of permutations/entropy (>25 000 permutations of the 2-2-3-3 content within the 10-mer block) and, therefore, do not benefit the scoring and ranking. FIG. 23b plots the content score as consecutive blocks have been analyzed (1 through 12 total blocks). It can be seen that the correct OXA gene from the MEGARes database is identified based on its top content score ranking well within the 12 blocks that are shown. The OXA gene begins to separate itself in ranking from the other database genes after merely the fifth block was analyzed, and it becomes easily identifiable after the twelfth block. The fifth block corresponds to only 0.063 or 6.3% coverage of the gene (50 nucleotides analyzed in the five 10-mer blocks, out of 789 total nucleotides in the resistance gene sequence), while the twelfth block corresponds to 0.152 or 15.2% coverage of the gene. Also shown in FIG. 23b is the specificity or how significantly the gene database can be narrowed as consecutive blocks have been analyzed. We see that >90% of the total MEGARes genes in the database can be eliminated after merely the first block is analyzed.

Extending diagnostic applications further, we ran our measured gene blocks through the algorithm again after substituting the MEGARes database for the P. aeruginosa reference genome PAO1 containing the OXA β-lactamase (class D) gene. This analysis indicates the ability to confirm pathogens and specific strains responsible for the infection. It also shows the robustness of the content-scoring algorithm in identifying specific genes in the background of an entire microbial genome. FIG. 23c plots the content score from the algorithm as consecutive gene blocks have been analyzed. Just as with the MEGARes resistance database, the correct OXA gene from the P. aeruginosa genome database is identified based on its top content score ranking within the 12 blocks. Additionally, high specificity is seen as >90% of the total genes in the database can be eliminated after merely the first block is analyzed. These results demonstrate the potential for using our optical sequencing platform as a diagnostic technique for profiling MDR pathogens. The results shown here for a single β-lactamase gene in P. aeruginosa can be extended to other resistance genes from pathogenic microbial strains without any changes in the experimental setup, ultimately providing the broad-spectrum detection needed for directing appropriate and timely treatments in a clinical setting.

### Example 16: Data Analysis

Entropy Screening in the BOCS Algorithm:

The most significant spikes in raw probabilities occur when the number of permutations for a particular k-mer block is low (i.e., the value k!/(A! G! C! T!) is low). Preferably analyzing these ‘low entropy’ blocks before others therefore enhances the BOCS algorithm by allowing for genetic biomarker identification at lower coverages, in a process the present inventors call entropy screening.

Thresholding in the BOCS Algorithm:

As more k-mer blocks are analyzed and content scores become compounded, genes within the biomarker database that have probabilistically become irrelevant need to be eliminated. For the case of analyzing k-mer blocks from a single gene at single coverage and no errors, genes can be eliminated when no content matches for a block occur. However, this elimination scheme cannot be implemented in the presence of errors, higher coverages, or the case of multiple genes comprising the k-mer blocks as it will lead to significant decreases in accuracy. To account for this, the present inventors implemented a thresholding system within BOCS to remove genes with lowest probability ranks after each consecutive round of block analyses. Thresholding is based on the rank of the content score, as well as each of the individual probability factors, and can be multiplied by a factor to increase/decrease the sensitivity of the eliminations being made.

Accounting for Special Characters in the Genetic Databases:

Some genetic biomarker database FASTA files contain special nucleic acid code characters (e.g., N signifies that either A, G, C, or T can be substituted into the sequence at that location). When performing content-based sequence alignment, this creates multiple possibilities for content within the two sequences being aligned (the k-mer block and genetic biomarker sequence). To account for these special characters, the BOCS algorithm tests all possible substitutions of A, G, C, and T for the character code used, and a match is awarded if any of the possible substitutions lead to equal content between block and gene sequence.

Making Genetic Biomarker Identifications:

The BOCS algorithm uses three levels for gene detection. In the order of most broad to most specific they include—class, sub-class, and specific gene. For example, a gene leading to resistance of beta-lactam antibiotics could have a class: class A beta-lactamase, sub-class: TEM, and specific gene: TEM-x,y,z (where x, y, z are specific mutations of TEM). Based on the level of phylogeny present in the genetic biomarker database, some or all of these classes are used. Each of these levels are tracked in terms of content score ranking throughout the k-mer blocks analysis and an identification can be made for each level. Identification is determined as the point where a gene within the database adopts one of the n-highest content scores (for n genes comprising the blocks) and remains there and/or separates itself probabilistically from the rest. False positives arise when genes other than the selected gene(s) meet this identification criterion.

Implementing a BOCS simulation: To generate large amounts of data on which to benchmark the BOCS algorithm without the need for experimental data, the present inventors built the BOCS algorithm into a simulation. The simulation uses gene sequences from a biomarker database to create k-mer blocks of A-G-C-T content as would be output from high-throughput BOS experiments. The simulated BOS reads are then run through the BOCS algorithm against the biomarker database. The goal of the simulation is to see how well the BOCS algorithm can identify the correct gene (out of all others in the database) using merely randomized k-mer blocks of A-G-C-T content. A specific gene from the database can be pulled or a random gene can be selected. The k-mer block lengths, gene coverage, and the number of errors within the blocks can all be set.

Simulating DNA k-Mer Blocks:

Blocks of DNA k-mer content within the BOCS simulation are generated from one (or more, based on simulation inputs) of the gene sequences within the biomarker database being used. Prior to fragmenting a gene sequence into k-mer blocks, random errors can be added at any specified rate. The gene sequence is split into k-mers based on the set value of k and whether k-mers are to be of constant length or variable length. For the variable length setting, lengths are randomly chosen from a normal distribution centered around the set value for k (with restrictions limiting the length to deviate no more than ±4). Note that the first and last fragments of the gene sequence can deviate from the settings in order to include the entire gene. After errors have been added to the sequence and the gene has been split into k-mers, fractional content for each k-mer is calculated and logged. This process is repeated for however many genes are selected for the analysis and for whatever integer the coverage is set to (for each additional +1× coverage, split locations for the blocks are different). The k-mer block contents for all genes selected for the analysis and all coverages are combined into a single randomized pool to be introduced into the BOCS algorithm. For each repeat simulation, split locations for the k-mer blocks and their randomized ordering will vary.

Simulation inputs/outputs: The following inputs can be set and tuned when running the BOCS simulation (see the Supplementary Information for more details):
Genetic biomarker database—any genetic database in FASTA format
How simulated k-mers are split (from the overall gene)—at a constant or variable length for k
Average length of k-mers
The overall coverage of the gene present throughout all blocks
The number of genes comprising the blocks
Error rate within the blocks
A penalty score is given to genes within the database when no matches to a block are observed
Multipliers for how sensitive genes within the database are to be eliminated
Entropy screening method—a randomized or idealized fashion
The set-point for what is considered low entropy

The BOCS simulation outputs a text file with the following data used for analysis (see the Supplementary Information for more details):
Simulation runtime, all inputs, and selected gene (gene from the database used to create k-mer blocks)
All k-mer blocks sequence and content, as well as the randomized order in which they were analyzed in the BOCS algorithm
Gene coverage as blocks are analyzed
Specificity as blocks are analyzed
Classification of the top-ranked genes within the database
Content scores for all genes in the database

Gene Databases:

The following exemplary gene databases may be applicable to the BOCS system described herein:
1. MEGARes—Antibiotic resistance genes Lakin, S. M.; Dean, C.; Noyes, N. R.; Dettenwanger, A.; Ross, A. S.; Doster, E.; Rovira, P.; Abdo, Z.; Jones, K. L.; Ruiz, J.; et al. MEGARes: An Antimicrobial Resistance Database for High Throughput Sequencing. Nucleic Acids Res. 2017, 45 (D1), D574-D580.
2. COSMIC—Cancer gene/somatic mutations Forbes, S. A.; Beare, D.; Gunasekaran, P.; Leung, K.; Bindal, N.; Boutselakis, H.; Ding, M.; Bamford, S.; Cole, C.; Ward, S.; et al. COSMIC:Exploring the World's Knowledge of Somatic Mutations in Human Cancer. Nucleic Acids Res. 2015, 43 (D1), D805-D811.
3. Genetic disease genes (custom compiled) Database contents:
   Achondroplasia: FGFR3
   Alpha-1 antitrypsin deficiency (AATD): SERPINA1
   Antiphospholipid syndrome (APS): ADAMTS-13
   Autism: ADNP, ANK2, ARID1B, ASXL3, CACNA1H, CHD2, CHD8, CNTN4, CNTNAP2, CTNND2, DYRK1A, GABRB3, GRIN2B, KDM5B, MECP2, MYT1L, NLGN3, NRXN1, POGZ, PTCHD1, PTEN, RELN, SCN2A, SHANK2, SHANK3, SYNGAP1, TBR1, RPL10,
   NLGN4X, SNRPN
   Autosomal dominant polycystic kidney disease: PKD1, PKD2
   Breast cancer: BRCA1, BRCA2, PALB2, TP53, PTEN, ATM, CDH1, CHEK2, NBN, NF1, STK11, BARD1, BRIP1, CASP8, CTLA4, CYP19A1, FGFR2, H19, LSP1, MAP3K1,
   MRE11, RAD51, RAD51C, TERT, TOX3, XRCC2, XRCC3
   Charcot-Marie-Tooth: GARS
   Colon cancer: APC, MSH2, MLH1, PMS2, MSH6, PMS1
   Cri du chat: CTNND2, chromosome 5
   Crohn's disease: ATG16L1, IL23R, IRGM, NOD2, HLA-DRB1, 1L10, 1L12B, JAK2,
   LRRK2, MUC2, SLC22A4, SLC22A5, STAT3, TYK2
   Cystic fibrosis: CFTR
   Dercum disease (a.k.a. Adiposis dolorosa): cause unknown, associated genes unknown
   Down syndrome: chromosome 21
   Duane syndrome: CHN1, SALL4
   Duchenne muscular dystrophy: DMD
   Factor V Leiden thrombophilia: F5
   Familial hypercholesterolemia: APOB, LDLR, LDLRAP1, PCSK9
   Familial Mediterranean fever: MEFV, SAA1
   Fragile X syndrome: FMR1
   Gaucher disease: GBA
   Hemochromatosis: HAMP, HFE, HJV, PNPLA3, SLC40A1, TFR2
   Hemophilia: F8, F9
   Holoprosencephaly: DISP1, FGF8, FOXH1, GLIZ, NODAL, PTCH1, SHH, SIX3, TDGF1,
   ZIC2
   Huntington disease: HTT
   Klinefelter syndrome: chromosome x
   Marfan syndrome: FBN1
   Myotonic dystrophy: CNBP, DMPK
   Neurofibromatosis: NF1, NF2
   Noonan syndrome: A2ML1, BRAF, KRAS, LZTR1, MAP2K1, NRAS, PTPN11, RAF1,
   RASA2, RIT1, RRAS, SOS1, SOS2
   Osteogenesis imperfecta: COL1A1, COL1A2, CRTAP, P3H1
   Parkinson's disease: ATP13A2, GBA, LRRK2, PARK7, PRKN, SNCA, UCHL1, VPS35
   Phenylketonuria: PAH
   Porphyria: ALAD, ALAS2, CPDX, FECH, HFE, HMBS, PPDX, UROD, UROS
   Progeria syndrome: LMNA
   Prostate cancer: AR, BRCA1, BRCA2, CD82, CDH1, CHEK2, EHBP1, ELAC2, EP300, EPHB2, EZH2, FGFR2, FGFR4, GNMT, HNF1B, HOXB13, IGF2, ITGA6, KLF6, LRP2, MAD1L1, MED12, MSMB, MSR1, MXI1, NBN, PCNT, PTEN, RNASEL, SRD5A2,
   STAT3, TGFBR1, WRN, WT1, ZFHX3
   Retinitis pigmentosa: ABCA4, BEST1, C2orf71, CA4, CERKL, CLRN1, CNGA1, CNGB1, CRB1, CRX, EYS, FAM161A, FSCN2, GUCA1B, IDH3B, IMPDH1, IMPG2, KLH7, LRAT, MERTK, NR2E3, NRL, PDE6A, PDE6B, PDE6G, PRCD, PROM1, PRPF8, PRPF3, PRPF31, PRPH2, RBP3, RDH12, RGR, RHO, RLBP1, ROM1, RP1, RP2, RP9, RPE65, RPGR, SAG, SEMA4A, SNRNP200, SPATA7, TOPORS, TTC8, TULP1, USH2A, WDR19,
   ZNF513
   Severe combined immunodeficiency (SCID): IL2RG, JAK3, ZAP70
   Sickle cell disease: HBB
   Skin cancer: CDKN2A, CDK4, CDK6, BAP1, BRCA2, PTCH1, PTCH2
   Spinal muscular atrophy: DYNC1H1, SMN1, SMN2, UBA1, VAPB
   Tay-Sachs disease: HEXA
   Thalassemia: HBA1, HBA2, HBB, ATRX
   Trimethylaminuria: FMO3
   Turner syndrome: SHOX
   Velocardiofacial syndrome: COMT, TBX1, chromosome 22
   WAGR syndrome: BDNF, PAX6, WT1, chromosome 11
   Wilson disease: ATP7B, PRNP

Running the BOCS Simulation:

The following options for inputs/settings may available in certain embodiments of the BOCS system. Within the main text figures, tables are shown summarizing the important inputs that were used for each of the simulations. These include 3, 4, 5, 6, 7, 8, 9 below. The other inputs are not shown in the main text figures, and are merely user options dictating database options, file locations, output settings, and figure displays for further analysis.
1. Choose database—Specify the (1) database type and (2) name of the file. Note that if deviating from the three built-in database types, coding changes must be made. The file must be in the location ‘Data/{database_name}/lasta’, and the file must be in the .fasta format. Variables to set . . .
   g_database
   database_name
2. Output file location—Specify the folder location for the output .txt file to be written. Variable to set . . .
   file_output_loc
3. Length of k-mers—Specify (1) the k-mer splitting method and the (2) k-mer length. Variables to set . . .
   kmer_split_method—Choose ‘constant’ for k-mers of the same length or ‘variable’ for k-mers of varying length centered around the avg specified by kmer length, picked from a normal distribution with stdev=2.
   kmer length
4. Coverage per nucleotide—Specify the coverage at which each nucleotide in the sequence is seen in the blocks. Breaks for blocks are made in different locations for each additional +1× coverage. Variable to set . . .
   gene_coverage (must be an integer)
5. Number of genes and select genes—Specify (1) the number of genes from which the blocks are comprised and (2) the number(s) within the database of the specific genes (if any) to use. The genes will be split into blocks and randomized in a batch with blocks from all. Variables to set . . .
   num_genes (must be an integer)
   sel_genes—Enter the numbers in an array for the specific genes within the database being used. This is an optional input, as random gene(s) will be selected if nothing is entered. The number of entries in the array must match the value entered for num_genes.
6. Errors—Specify (1) whether random errors should be inserted and (2) the rate at which they are seen. Note that the specified error rate corresponds to the number of random point errors, which is actually only half of the error rate observed in content-based sequencing. So, the actual error rate in the block optical method is double the entered value. Variables to set . . .
   error mode—Choose ‘on’ or ‘off’(in the ‘off’ state, the err_rate is neglected)
   err_rate
7. Penalty score—Specify the score given to genes when no matches are found for a specific block. It is suggested that a value of 0.1 is used for starting and for most normal analyses. Variable to set . . .
   penalty score
8. Thresholding parameters—Specify (1) the multiplier to be multiplied to each of the standard thresholding trends and (2) which of the probability factors to use for thresholding. Variables to set . . .
   thresh_multiplier—This can be thought of as a sensitivity, where values >1 correspond to a LESS sensitive state (i.e., more genes remain in consideration after each block is analyzed), and values <1 correspond to a MORE sensitive state (i.e., fewer genes remain in consideration after each block is analyzed).
   thresh_prob_facts_CS—Choose I/O (on/off)
   thresh_prob_facts_F1—Choose I/O (on/off)
   thresh_prob_facts_F2—Choose I/O (on/off)
   thresh_prob_facts_F3—Choose I/O (on/off)
   thresh_prob_facts_F4—Choose I/O (on/off)
   thresh_prob_facts_F5—Choose I/O (on/off)
   thresh_prob_facts_F6—Choose I/O (on/off)
9. Entropy screening—Specify (1) the entropy screening mode and (2) the threshold for what is considered ‘high entropy’. Variables to set . . .
   entropy_screening_mode—Options are ‘rand’ for random entropy screening in whichever order the blocks are randomized, ‘ideal’ for entropy screening idealized from lowest to highest, and ‘none’ for no entropy screening.
   perms_thresh—It is suggested to use 10000 as the marker for high entropy since there is a natural break in possible entropy values near this number.
10. Analysis/Troubleshooting/Output options—Specify (1) what kind of analysis is being done, (2) if factor analysis is needed (i.e., figures are displayed for each factor comprising the content score after each block), and (3) the level at which to track gene class. Variables to set . . .
   analysis_type—Select ‘standard’ for normal operation and output or ‘benchmarking’ for extra output including all of the factor values for all of the genes in the database, for establishing new thresholding trends.
   disp_fact_figs—Choose ‘yes’ or ‘no’
   tracking_level—This is the number of unique sub-classes of genes with the top content scores after each consecutive block is analyzed. The number used here should be increased as more genes are combined and helps in analyzing the level of identification of the selected genes (i.e., positive and false positive identifications).

BOCS Output

The following sections may be output in the results .txt file. The .txt files can be analyzed for overall simulation performance and metrics such as coverage at which the selected gene(s) was identified, accuracy, and false positives.
1. Runtime—Displays the runtime of the content mapping scoring section (i.e., BOCS algorithm)
2. Inputs—Displays all user inputs and options in the following exemplary order . . .
   a. g_database
   b. database_name
   c. file_output_loc
   d. kmer_split_method
   e. kmer_length
   f. gene_coverage
   g. num_genes
   h. sel_genes
   i. err_mode
   j. err_rate
   k. penalty_score
   1. thresh_multiplier
   m. array of thresh_prob_facts_X, where X=CS, F1, F2, F3, F4, F5, F6
   n. entropy_screening_mode
   o. perms_thresh
   p. analysis_type
   q. tracking_level
4. Selected genes data—Information on the selected genes in the study in the order . . .
   a. Gene number in database
   b. Gene sub-class
   c. Gene class (if the resistance database)
   d. Full gene header/name
   e. Gene sequence
   f. Gene sequence with errors (if err mode is ‘on’)
5. Blocks for each selected gene(s)—For each coverage of each gene, the columns show . . .
   a. Block number
   b. Block sequence
   c. A content
   d. G content
   e. C content
   f. T content
6. Randomized blocks—For the combined genes and all coverages, the columns show . . .
   a. Block number
   b. Gene to which the block belongs
   c. Block sequence
   d. Block entropy
   e. A content
   f. G content
   g. C content
   h. T content
7. Blocks ordered for the final analysis—For the combined genes and all coverages, the columns show . . .
   a. Block number
   b. Gene to which the block belongs
   c. Block sequence
   d. Block entropy
   e. A content
   f. G content
   g. C content
   h. T content
8. Increasing coverage—Coverage is shown for each individual gene and the overall coverage, with columns showing . . .
   a. Block number
   b. Coverage for individual genes (each with its own column, 1 . . . num_genes)
   c. Coverage for all genes overall
9. Specificity—Specificity for the overall algorithm with columns . . .
   a. Block number
   b. Remaining genes (integer)
   c. Specificity (fraction in range 0-1)
10. Class analysis—The sub-classes and classes (depending on the database used) of the top content scoring genes after each block is analyzed, with columns in the following order . . .
   a. Block number
   b. Whether the specific selected gene(s) is identified−1=′yes' and 0=′no′, there is a column for each selected gene (1 . . . num_genes)
   c. The sub-classes with top content scores (1 . . . tracking_level)
   d. The content scores for the top sub-classes (1 . . . tracking_level)
   e. If the resistance database is being used, The classes with top content scores (1 . . . tracking_level)
   f. If the resistance database is being used, the content scores for the top classes (1 . . . tracking_level)
11. All probability factors (analysis_type=‘benchmarking’ mode only)—All probability factors (and some slope analyses) are output for each block for each gene in the database in a matrix with dimensions (number genes×number blocks+1), with columns . . .
   a. Gene number
   b. Cumulative probability factor (or slope analysis) for each block (1 . . . number of blocks)
12. All content scores—All content scores are output for each block for each gene in the database in a matrix with dimensions (number genes×number blocks+1), with columns . . .
   a. Gene number
   b. Content score (1 . . . number of blocks)
13. Content scores extracted for the selected genes—Content scores with columns . . .
   a. Block number

Synthesis of Positively-Charged Silver Nanoparticles (Ag NPs).

The synthesis protocol was adapted from van Lierop et al. Prior to synthesis, all glass vials were left to soak in the PEI solution (0.4% v/v) overnight followed by extensive rinsing with ultrapure DI water. For Ag NPs, silver nitrate solution (40 μL, 0.5 M) and spermine tetrahydrochloride solution (14 μL, 0.1 M) were mixed with ultrapure DI water (20 mL) and stirred for 20-30 min in the dark. After 20-30 min, sodium borohydride solution (500 μL, 0.01 M) was spiked into the mixture (with continued stirring for 5-10 min). Ag NP colloids were allowed to sit overnight in the dark (at room temperature), and the sediment at the bottom of the vial was then discarded.

Sample Preparation:

Prior to use, the Ag NPs were cleaned by collection with centrifugation at 9,000 rpm for 10 min, followed by redispersion in ultrapure DI water at half the original volume. Following mixture with DNA/RNA/amino acids (described below), the Ag NPs-analyte solution was centrifuged at 8,500 rpm for 5 min, 4/5 volume of supernatant was removed, and the sedimented sample was resuspended. Specific procedures for the different bio-analytes are described below.
DNA/RNA—Samples were prepared by mixing DNA/RNA oligomer solution (5 μL, 1 μM in ultrapure DI water or TAE buffer for the epigenetic marker oligomers) with Ag NPs colloidal solution (500 μL), for a final DNA/RNA concentration of ˜10 nM. The DNA/RNA-Ag NPs mixture was allowed to equilibrate for at least 20 min, followed by a second centrifugation step and a quick sonication prior to measuring.
Amino acids—Samples were prepared by mixing amino acid oligomer solution (5 μL, 10 μM in ultrapure DI water with minimal DMSO) with Ag NPs colloidal solution (500 μL), for a final oligomer concentration of ˜100 nM. The oligomer-Ag NPs mixture was allowed to equilibrate for ˜5-20 min, followed by addition of magnesium sulfate at 0.1 M. A second centrifugation step and a quick sonication was performed prior to measuring.

SERS Measurements:

SERS measurements were collected with a 532 nm 40 mW laser from Thorlabs, Inc. (diode-pumped solid state, operated at 15-20 mW) focused on the colloidal sample through a Zeiss Observer.Alm microscope with 50× objective, and spectra were collected with a Princeton Instruments Acton SpectraPro SP-2500 spectrometer with PIXIS 100 CCD camera at 30 s exposure time, 10 accumulations.

Signal Processing and Normalization:

Signal processing and normalization including cosmic ray removal, average smoothing, and baseline subtraction was described in Korshoj, L. E.; Nagpal, P. Diagnostic Optical Sequencing. ACS Appl. Mater. Interfaces 2019, 11 (39), 35587-35596, the entirety of which is incorporated herein by reference, and specifically materials and methods).

Peak Analysis with p-Value Statistics:

The difference in Raman signal between the DNA and RNA nucleobases was quantified with a p-value analysis on the intensity values observed for all distinct signature peaks. To generate p-values, t-tests (two-sample assuming equal variances) were performed with the intensities of each nucleobase Raman signal for each of the signature peaks. For RNA, the p-values for the U signature were generated with a χ² analysis on a combination of two peaks in accordance with Fisher's method.

TABLE 1
Raman spectroscopy peaks and vibrational modes for nucleobases

| | Shift | |
|---|---|---|
| Peak | (cm⁻¹) | Assignment |
| A₁ | 612 | N—C—C bend |
| A₂ | 782 | C—C stretch |
| A₃ | 1150 | C2—N1═C6 bend |
| | | C5—N7═C8 stretch |
| A₄ | 1218 | C—NH₂ stretch |
| A₅ | 1320 | C—N and C═N stretch |
| T₁ | 645 | N—C—C and C—H bend |
| T₂ | 767 | C5—CH₃ stretch |
| T₃ | 806 | C4—C5 stretch |
| T₄ | 1045 | N—C—H bend |
| T₅ | 1112 | CH₃ rocking |
| C₁ | 572 | C2—N3═C4 and N1—C2—N3 bend |
| C₂ | 803 | Breathing mode |
| C₃ | 1238 | C4—N4 stretch |
| C₄ | 1379 | C═C—H bend |
| G₁ | 1167 | C8—H in plane bend |
| G₂ | 1282 | C5—N7 and C4—N9 stretch |
| G₃ | 1366 | C8—NH and C8—H bend |
| | | C8—N stretch |
| G₄ | 1421 | N7═C8—H bend |
| U₁ | 436 | C2—O7 and C4—O8 bend |
| U₂ | 556 | C2—N3—C4 C5—C6—N1 deformation (squeezing) |
| U₃ | 673 | C2—N3—C4, O4—C4—C5, and N1C2O deformation |
| | | (squeezing) |
| U₄ | 765 | N1—C2—N3 deformation (wagging) |
| U₅ | 1162 | N1—H, C6—H, and C5—H bend |
| | | C6—N1 stretch |
| U₅ | 1354 | N3—H, C5—H, and C6—H bend |
| 5mC₁ | 1168 | C—O stretch |
| 5mC₂ | 1269 | C4—N4 stretch |
| 5mC₂ | 1379 | C═C—H bend |

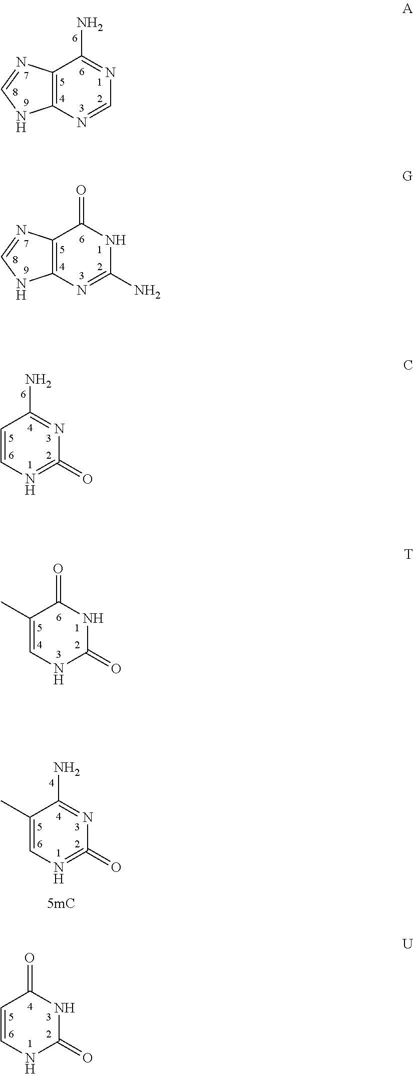

TABLE 2
Raman spectroscopy peaks and vibrational modes for amino acids

| | Shift | |
|---|---|---|
| Peak | (cm⁻¹) | Assignment |
| His₁ | 689 | Imidazole out-of-plane bend |
| His₂ | 710 | Imidazole ring breathing |
| His₃ | 736 | C—H bend |
| Met₁ | 660 | C—S stretch |
| Met₂ | 768 | CH2 rocking |
| Met₃ | 895 | C—C stretch |
| Met₄ | 1062 | C—N stretch |
| Ser₁ | 406 | Skeletal deformation |
| Ser₂ | 1202 | CH2 twist |
| Ser₃ | 1234 | C—O—H bend |
| Tyr₁ | 372 | Ring deformation |
| Tyr₂ | 795 | Ring breathing |

TABLE 3
Raman spectroscopy peaks

| | Shift | |
|---|---|---|
| Peak | (cm⁻¹) | Assignment |
| A1 | 340 | Hydrogen bonding |
| A2 | 537 | C—C═C bend |
| A3 | 622 | N—C—C bend |
| A4 | 737 | C—C and C—N in-phase stretch |
| A5 | 971 | N—C═N bend |
| A₆ | 1045 | C—N—C bend |
| A₇ | 1140 | C2—N1═C6 bend |
| | | C5—N7═C8 stretch |
| A₈ | 1320 | C—N stretch |
| A₉ | 1350 | C═N stretch |
| a₁ | 841 | skeletal mode, in-plane |
| a₂ | 1167 | CH bend, in-plane |
| T₁ | 304 | OH ^{. . .} O bend |
| T₂ | 467 | N—C═C bend |
| T₃ | 647 | N—C—C bend |
| T₄ | 737 | C5—CH₃ stretch |
| T₅ | 832 | C4—C5 stretch |
| T₆ | 1017 | C—N—C bend |
| T₇ | 1059 | N—C—H bend |
| t₁ | 589 | C—C═C bend |
| t₂ | 953 | C5—C—H bend |
| C₁ | 395 | N3—C2═O and N1—C2═O |
| | | bend |
| C₂ | 468 | C2—N1—C6 and N3═C4—C5 |
| | | bend |
| C₄ | 538 | C—C═C and N3═C4—N4 bend |
| C₅ | 558 | C2—N3═C4 and N1—C2—N3 |
| | | bend |
| C₆ | 611 | C═O in-phase stretch |
| C₈ | 788 | Breathing mode |
| C₁₀ | 973 | C4—C5 stretch |
| c₂ | 715 | C5—C4—N4 bend |
| c₃ | 1000 | C4—C5—H in-plane bend |
| c₄ | 1028 | N1—C6—H in-plane bend |
| G₁ | 402 | C═O bend |
| G₂ | 511 | N9—C4=C5 and N7—C═C4 |
| | | bend |
| G₃ | 604 | C═C═C bend |
| G₄ | 648 | Breathing mode |
| G₆ | 847 | C—C stretch |
| G₇ | 931 | N—C═N and N—C—N bend |
| G₁₁ | 1226 | C2—NH2 stretch |
| g₁ | 548 | N3—C4═C5 bend |
| g₃ | 866 | N9—H out-of-plane bend |

TABLE 4
FTIR spectroscopy peaks

| | Wavenumber | |
|---|---|---|
| Peak | (cm⁻¹) | Assignment |
| α₂ | 727 | C—C and C—N in-phase bend |
| α₃ | 807 | C—C stretch |
| α₄ | 869 | N9—H out-of-plane bend |
| α₅ | 952 | N—C═N bend |
| α₇ | 1129 | C2—N1═C6 bend |
| | | C5—N7═C8 stretch |
| α₉ | 1371 | C2—H and C8—H out-of-plane bend |
| | | N═C—H bend |
| α₁₀ | 1460 | Imidazole ring stretch |
| α₁₁ | 1507 | C—N9—H bend |
| α₁₂ | 1620 | C═N and C═C stretch |
| α₁₃ | 1650 | NH₂ bend |
| τ₂ | 861 | N—H out-of-plane bend |
| τ₇ | 1227 | C—N stretch |
| τ₉ | 1511 | N1—H and N3—H bend |
| τ₁₂ | 1750 | C4═O and C2═O stretch |
| χ₁ | 813 | N—H out-of-plane bend |
| χ₃ | 1077 | NH₂ rocking |
| χ₄ | 1235 | C4—N4 stretch |
| χ₅ | 1361 | C═C—H bend |
| χ₆ | 1458 | C4—N3 and C2—N3 stretch |
| χ₇ | 1519 | C4═N3 and C4—N4 stretch |
| χ₈ | 1626 | C5═C6 stretch |
| χ₉ | 1708 | NH₂ bend |
| γ₂ | 712 | Ring bend |
| γ₃ | 804 | N1—H bend |
| γ₄ | 860 | C—C stretch |
| γ₆ | 1056 | NH₂ rocking |
| γ₁₀ | 1493 | N7═C8 and C8—C9 stretch |
| γ12 | 1660 | C═O stretch |
| γ13 | 1698 | C═O stretch |
| | | NH2 bend |

TABLE 5
Calibration Blocks (SEQ ID NOs. 1-14)

| block | | Content | | | |
|---|---|---|---|---|---|
| name | sequence | A | G | C | T |
| Cal_1 | AAAAAAAAAA | 1 | 0 | 0 | 0 |
| Ca1_2 | GGGGGGGGGG | 0 | 1 | 0 | 0 |
| Cal_3 | CCCCCCCCCC | 0 | 0 | 1 | 0 |
| Cal_4 | TTTTTTTTTT | 0 | 0 | 0 | 1 |
| Cal_5 | AAAGAAAACA | 0.8 | 0.1 | 0.1 | 0 |
| Cal_6 | GGGTGGGAGG | 0.1 | 0.8 | 0 | 0.1 |
| Cal_7 | CCTCCCACCC | 0.1 | 0 | 0.8 | 0.1 |
| Cal_8 | TGTTTTCTTT | 0 | 0.1 | 0.1 | 0.8 |
| Cal_9 | AGAATAGAAT | 0.6 | 0.2 | 0 | 0.2 |
| Cal_10 | CGGAGGAGCG | 0.2 | 0.6 | 0.2 | 0 |
| Cal_11 | CGCTCCGCCT | 0 | 0.2 | 0.6 | 0.2 |
| Cal_12 | CTTCTATTAT | 0.2 | 0 | 0.2 | 0.6 |
| Cal_13 | AACGCATCCA | 0.4 | 0.1 | 0.4 | 0.1 |
| Cal_14 | GTGCGATTGT | 0.1 | 0.4 | 0.1 | 0.4 |

TABLE 6
Gene Blocks (SEQ ID NOs. 15-28)

| block | | Content | | | |
|---|---|---|---|---|---|
| name | sequence | A | G | C | T |
| Gen_1 | CCCACTTTCT | 0.1 | 0 | 0.5 | 0.4 |
| Gen_2 | ACGAGGTTCT | 0.2 | 0.3 | 0.2 | 0.3 |
| Gen_3 | GCGCAGGGAG | 0.2 | 0.6 | 0.2 | 0 |
| Gen_4 | GATCAGCGCG | 0.2 | 0.4 | 0.3 | 0.1 |
| Gen_5 | CCCCTCCTCT | 0 | 0 | 0.7 | 0.3 |
| Gen_6 | GGTGGCGAAC | 0.2 | 0.5 | 0.2 | 0.1 |
| Gen_7 | AAGCGCAACG | 0.4 | 0.3 | 0.3 | 0 |
| Gen_8 | CTTCGTCCTC | 0 | 0.1 | 0.5 | 0.4 |
| Gen_9 | AGCGGCTCTA | 0.2 | 0.3 | 0.3 | 0.2 |
| Gen_10 | GGTGGGTGGG | 0 | 0.8 | 0 | 0.2 |
| Gen_11 | GACCGGGAGC | 0.2 | 0.5 | 0.3 | 0 |
| Gen_12 | GCCAGGTTGT | 0.1 | 0.4 | 0.2 | 0.3 |
| Gen_13 | GCCAATGTCT | 0.2 | 0.2 | 0.3 | 0.3 |
| Gen_14 | AAGCCCCAGC | 0.3 | 0.2 | 0.5 | 0 |
| Gen_15 | CCGTGCGCGC | 0 | 0.4 | 0.5 | 0.1 |

TABLE S1
70 randomly-selected resistance genes

| | Gene | | | |
|---|---|---|---|---|
| | data- | | | |
| | base | Sub- | | |
| No. | No. | class | Class | Full gene name (from MEGARes database) |
| 1 | 4 | VANZA | VanA-type_accessory_protein | 959\|M97297.1\|TRNVAN\|Glycopeptides\|VanA-type_accessory_protein\|VANZA |
| 2 | 52 | VANWG | VanG-type_accessory_protein | Gly\|VanW-G_1_AY271782\|Glycopeptides\|VanG-type_accessory_protein\|VANWG |
| 3 | 62 | CTX | Class_A_betalactamases | Bla\|CTX-M-9\|AF174129\|1-876\|876\|betalactams\|Class_A_betalactamases\|CTX |
| 4 | 68 | MEFA | Macrolide_resistance_efflux_pumps | MLS\|NC_023287.1.18156494\|MLS\|Macrolide_resistance_efflux_pumps\|MEFA |
| 5 | 76 | OXA | Class_D_betalactamases | Bla\|OXA-208\|FR853176\|1-825\|825\|betalactams\|Class_D_betalactamases\|OXA |
| 6 | 92 | CATB | Chloramphenicol_acetyltransferases | Phe\|FJ460181.2\|gene2\|Phenicol\|Chloramphenicol_acetyltransferases\|CATB\|RequiresSNPConfirmation |
| 7 | 162 | EREA | Macrolide_esterases | MLS\|ereA_4_AF512546\|MLS\|Macrolide_esterases\|EREA |
| 8 | 174 | TEM | Class_A_betalactamases | Bla\|TEM-59\|AF062386\|31-862\|832\|betalactams\|Class_A_betalactamases\|TEM |
| 9 | 193 | CML | Phenicol_efflux_pumps | 1597\|HQ713678.1\|HQ713678\|Phenicol\|Phenicol_efflux_pumps\|CML |
| 10 | 207 | NIMA | nim_nitroimidazole_reductase | Met\|nimH_1_FJ969397\|Metronidazole\|nim_nitroimidazole_reductase\|NIMA |
| 11 | 239 | DHFR | Dihydrofolate_reductase | Tmt\|dfrA1_2_AJ419168\|Trimethoprim\|Dihydrofolate_reductase\|DHFR\|RequiresSNPConfirmation |
| 12 | 246 | FOLP | Sulfonamide-resistant_dihydropteroate_synthases | Sul\|CP001581.1\|gene938\|Sulfonamides\|Sulfonamide- |
| | | | | resistant_dihydropteroate_synthases\|FOLP\|RequiresSNPConfirmation |
| 13 | 274 | PARC | Fluoroquinolone-resistant_DNA_topoisomerases | Flq\|NC_011586.7046300\|Fluoroquinolones\|Fluoroquinolone- |
| | | | | resistant_DNA_topoisomerases\|PARC\|RequiresSNPConfirmation |
| 14 | 276 | SHV | Class_A_betalactamases | gi\|28912444\|gb\|AY210887.1\|betalactams\|Class_A_betalactamases\|SHV |
| 15 | 284 | DFRA | Dihydrofolate_reductase | CARD\|phgb\|AM403715\|302-854\|ARO:3002857\|dfrA26\|Trimethoprim\|Dihydrofolate_reductase\|DFRA |
| 16 | 295 | SOXS | MDR_regulator | CARD\|pvgb\|NC_003197\|4503969-4504293\|ARO:3003383\|Salmonella\|Multi- |
| | | | | drug_resistance\|MDR_regulator\|SOXS\|RequiresSNPConfirmation |
| 17 | 321 | TSNR | Thiostrepton_23S_rRNA_methyltransferases | CARD\|phgb\|AL123456\|1853605- |
| | | | | 1854388\|ARO:3003060\|tsnr\|Thiostrepton\|Thiostrepton_23S_rRNA_methyltransferases\|TSNR |
| 18 | 413 | OMPD | Mutant_porin_proteins | Bla\|CP004022.1\|gene834\|betalactams\|Mutant_porin_proteins\|OMPD |
| 19 | 486 | LMRA | Lincomycin-resistant_lmrA | CARD\|pvgb\|AL009126\|290131-290698\|ARO:3003028\|lmrA\|MLS\|Lincomycin- |
| | | | | resistant_lmrA\|LMRA\|RequiresSNPConfirmation |
| 20 | 505 | CARB | Class_A_betalactamases | CARD\|phgb\|HF953351\|2461-3358\|ARO:3002255\|CARB-16\|betalactams\|Class_A_betalactamases\|CARB |
| 21 | 506 | ANT3-DPRIME | Aminoglycoside_O-nucleotidyltransferases | CARD\|phgb\|NC_010410\|3621491-3622283\|ARO:3002601\|aadA\|Aminoglycosides\|Aminoglycoside_O- |
| | | | | nucleotidyltransferases\|ANT3-DPRIME |
| 22 | 555 | QNRS | Quinolone_resistance_protein_Qnr | Flq\|QnrS6_1_HQ631376\|Fluoroquinolones\|Quinolone_resistance_protein_Qnr\|QNRS |
| 23 | 558 | VANWI | VanI-type_accessory_protein | CARD\|phgb\|NC_007907.1\|2195400-2196522\|ARO:3003724\|vanWI\|Glycopeptides\|VanI- |
| | | | | type_accessory_protein\|VANWI |
| 24 | 578 | TETX | Tetracycline_inactivation_enzymes | Tet\|tetX_1_GU014535\|Tetracyclines\|Tetracycline_inactivation_enzymes\|TETX |
| 25 | 652 | VGA | ABC_transporter | 105\|FR772051.1\|FR772051\|MLS\|ABC_transporter\|VGA |
| 26 | 682 | MOX | Class_C_betalactamases | Bla\|MOX-2\|AJ276453\|4620-5768\|1149\|betalactams\|Class_C_betalactamases\|MOX |
| 27 | 694 | ACT | Class_C_betalactamases | gi\|595583477\|gb\|KF992026.1\|betalactams\|Class_C_betalactamases\|ACT |
| 28 | 717 | VANRE | VanE-type_regulator | CARD\|phgb\|FJ872411\|43513-44203\|ARO:3002924\|vanRE\|Glycopeptides\|VanE-type_regulator\|VANRE |
| 29 | 749 | RMTB | 16S_rRNA_methyltransferases | CARD\|phgb\|FJ483516.1\|0-252\|ARO:3000860\|rmtB\|Aminoglycosides\|16S_rRNA_methyltransferases\|RMTB |
| 30 | 778 | DHA | Class_C_betalactamases | gi\|698174199\|gb\|KM087854.1\|betalactams\|Class_C_betalactamases\|DHA |
| 31 | 789 | CMY | Class_C_betalactamases | Bla\|CMY-4\|AF420597\|1-1146\|1146\|betalactams\|Class_C_betalactamases\|CMY |
| 32 | 797 | FACT | Elfamycin_efflux_pumps | CARD\|phgb\|JQ768046\|7760-9440\|ARO:3001313\|facT\|Elfamycins\|Elfamycin_efflux_pumps\|FACT |
| 33 | 819 | OKP | Class_A_betalactamases | Bla\|OKP-A-3\|AM051140\|1-861\|861\|betalactams\|Class_A_betalactamases\|OKP |
| 34 | 973 | IMP | Class_B_betalactamases | 1570\|LC031883.1\|LC031883\|betalactams\|Class_B_betalactamases\|IMP |
| 35 | 1048 | VIM | Class_B_betalactamases | Bla\|VIM-37\|JX982636\|1-801\|801\|betalactams\|Class_B_betalactamases\|VIM |
| 36 | 1135 | PBP1B | Penicillin_binding_protein | CARD\|phgb\|NC_003098\|1886035-1888501\|ARO:3003044\|PBP1b\|betalactams\|Penicillin_binding_protein\|PBP1B |
| 37 | 1146 | MPHE | Macrolide_phosphotransferases | 1507\|unknown_id\|unknown_name\|MLS\|Macrolide_phosphotransferases\|MPHE |
| 38 | 1182 | VATE | Streptogramin_A_O-acetyltransferase | MLS\|vatE_3_AF153312\|MLS\|Streptogramin_A_O-acetyltransferase\|VATE |
| 39 | 1214 | OPRJ | MDR_mutant_porin_proteins | 155\|U57969.1\|PAU57969\|Multi-drug_resistance\|MDR_mutant_porin_proteins\|OPRJ |
| 40 | 1254 | FOSB | Fosfomycin_thiol_transferases | Fos\|Fcyn\|FosB\|AHLO01000073\|63139-63558\|417\|Fosfomycin\|Fosfomycin_thiol_transferases\|FOSB |
| 41 | 1271 | VPH | Viomycin_phosphotransferases | CARD\|phgb\|X02393\|96-960\|ARO:3003061\|viomycin\|Mycobacterium_tuberculosis- |
| | | | | specific_Drug\|Viomycin_phosphotransferases\|VPH |
| 42 | 1283 | SULI | Sulfonamide-resistant_dihydropteroate_synthases | Sul\|sul1_22_AY115475\|Sulfonamides\|Sulfonamide-resistant_dihydropteroate_synthases\|SULI |
| 43 | 1297 | TET40 | Tetracycline_resistance_ribosomal_protection_proteins | Tet\|JQ740052.1\|gene2\|Tetracyclines\|Tetracycline_resistance_ribosomal_protection_proteins\|TET40 |
| 44 | 1389 | CPXAR | MDR_regulator | Mdr\|CP000034.1\|gene3834\|Multi-drug_resistance\|MDR_regulator\|CPXAR |
| 45 | 1392 | AAC6-PRIME | Aminoglycoside_N-acetyltransferases | AGly\|Aac6-32\|EF614235\|2247-2801\|555\|Aminoglycosides\|Aminoglycoside_N-acetyltransferases\|AAC6-PRIME |
| 46 | 1422 | VGBB | Streptogramin_B_ester_bond_cleavage | CARD\|phgb\|AF015628\|398-1286\|ARO:3001308\|VgbB\|MLS\|Streptogramin_B_ester_bond_cleavage\|VGBB |
| 47 | 1440 | FOSC | Fosfomycin_phosphorylation | CARD\|phgb\|Z33413\|386-935\|ARO:3000380\|FosC\|Fosfomycin\|Fosfomycin_phosphorylation\|FOSC |
| 48 | 1535 | LNUA | Lincosamide_nucleotidyltransferases | MLS\|AM399080.1\|gene2\|MLS\|Lincosamide_nucleotidyltransferases\|LNUA |
| 49 | 1569 | PARE | Aminocoumarin-resistant_DNA_topoisomerases | ACou\|CP000675.2\|gene802\|Aminocoumarins\|Aminocoumarin- |
| | | | | resistant_DNA_topoisomerases\|PARE\|RequiresSNPConfirmation |
| 50 | 1695 | NDM | Class_B_betalactamases | CARD\|phgb\|FN396876\|2406-3219\|ARO:3000589\|NDM-1\|betalactams\|Class_B_betalactamases\|NDM |
| 51 | 1702 | SPG | Class_B_betalactamases | CARD\|phgb\|KP109680\|1254-2112\|ARO:3003720\|SPG-1\|betalactams\|Class_B_betalactamases\|SPG |
| 52 | 1753 | VEB | Class_A_betalactamases | Bla\|VEB-1_1_HM370393\|betalactams\|Class_A_betalactamases\|VEB |
| 53 | 1953 | LNUB | Lincosamide_nucleotidyltransferases | MLS\|LnuB\|AJ238249\|127-930\|804\|MLS\|Lincosamide_nucleotidyltransferases\|LNUB |
| 54 | 2026 | ERMA | 23S_rRNA_methyltransferases | 126\|AJ579365.1\|AJ579365\|MLS\|23S_rRNA_methyltransferases\|ERMA |
| 55 | 2357 | SULII | Sulfonamide-resistant_dihydropteroate_synthases | Sul\|sul2_12_AF497970\|Sulfonamides\|Sulfonamide-resistant_dihydropteroate_synthases\|SULII |
| 56 | 2517 | TET37 | Tetracycline_inactivation_enzymes | CARD\|phgb\|AF540889\|0-327\|ARO:3002871\|tet37\|Tetracyclines\|Tetracycline_inactivation_enzymes\|TET37 |
| 57 | 2822 | EMRK | Multi-drug_efflux_pumps | CARD\|phgb\|D78168\|536-1592\|ARO:3000206\|emrK\|Multi-drug_resistance\|Multi-drug_efflux_pumps\|EMRK |
| 58 | 2999 | MPHB | Macrolide_phosphotransferases | 424\|D85892.1\|D85892\|MLS\|Macrolide_phosphotransferases\|MPHB |
| 59 | 3024 | VANYM | VanM-type_accessory_protein | 299\|FJ349556.1\|FJ349556\|Glycopeptides\|VanM-type_accessory_protein\|VANYM |
| 60 | 3041 | MECC | Penicillin_binding_protein | CARD\|phgb\|AB037671\|24420-26427\|ARO:3001209\|mecC\|betalactams\|Penicillin_binding_protein\|MECC |
| 61 | 3128 | TUFAB | EF-Tu_inhibition | Elf\|CP000647.1\|gene3761\|Elfamycins\|EF-Tu_inhibition\|TUFAB\|RequiresSNPConfirmation |
| 62 | 3176 | AMRB | Multi-drug_efflux_pumps | CARD\|phgb\|NC_002516\|2208168-2211306\|ARO:3002983\|amrB\|Multi-drug_resistance\|Multi- |
| | | | | drug_efflux_pumps\|AMRB |
| 63 | 3270 | IRI | Monooxygenase | CARD\|phgb\|U56415\|279-1719\|ARO:3002884\|iri\|Rifampin\|Monooxygenase\|IRI |
| 64 | 3314 | RPOB | Rifampin-resistant_beta- | Rif\|NC_002758.1120515\|Rifampin\|Rifampin-resistant_beta- |
| | | | subunit_of_RNA_polymerase_RpoB | subunit_of_RNA_polymerase_RpoB\|RPOB\|RequiresSNPConfirmation |
| 65 | 3332 | TET35 | Tetracycline_resistance_major_facilitator_superfamily_{—} | CARD\|phgb\|AF353562\|0- |
| | | | MFS_efflux_pumps | 1110\|ARO:3000481\|tet35\|Tetracyclines\|Tetracycline_resistance_major_facilitator_superfamily_MFS_efflux_pumps\|TET |
| | | | | 35 |
| 66 | 3370 | CFRA | Florfenicol_methyltransferases | MLS\|CfrA\|AM408573\|10028-11077\|1050\|Phenicol\|Florfenicol_methyltransferases\|CFRA |
| 67 | 3513 | BRP | Bleomycin_resistance_protein | CARD\|phgb\|NC_012547\|21239-21638\|ARO:3001205\|bleomycin\|Glycopeptides\|Bleomycin_resistance_protein\|BRP |
| 68 | 3613 | APH3-PRIME | Aminoglycoside_O-phosphotransferases | AGly\|APH-Stph\|HE579073\|1778413-1779213\|801\|Aminoglycosides\|Aminoglycoside_O-phosphotransferases\|APH3- |
| | | | | PRIME |
| 69 | 3697 | TETM | Tetracycline_resistance_ribosomal_protection_proteins | Tet\|tetM_6_M21136\|Tetracyclines\|Tetracycline_resistance_ribosomal_protection_proteins\|TETM |
| 70 | 3778 | IND | Class_B_betalactamases | Bla\|IND-11\|HM245379\|57-788\|732\|betalactams\|Class_B_betalactamases\|IND |

These 70 genes were used throughout all studies and simulations with the MEGARes antibiotic resistance database.

TABLE S2
Resistance genes simulations with no thresholding or entropy screening

| | | | Avg | StDev | Avg | StDev | | Identification level | | | | | |
|---|---|---|---|---|---|---|---|---|---|---|---|---|---|
| | Data- | Gene | block for | block for | coverage for | coverage for | | (fraction of trials) | | | | Avg | StDev |
| | base | sub- | identifi- | identifi- | identifi- | identifi- | Accu- | Specific | Sub- | | Incor- | false | false |
| No. | No. | class | cation | cation | cation | cation | racy | gene | class | Class | rect | positives | positives |
| 1 | 4 | VANZA | 15.240 | 7.655 | 0.310 | 0.156 | 1.000 | 1.000 | 0.000 | 0.000 | 0.000 | 0.000 | 0.000 |
| 2 | 52 | VANWG | 20.240 | 7.721 | 0.238 | 0.091 | 1.000 | 0.280 | 0.720 | 0.000 | 0.000 | 0.000 | 0.000 |
| 3 | 62 | CTX | 26.560 | 12.553 | 0.302 | 0.142 | 1.000 | 0.000 | 1.000 | 0.000 | 0.000 | 0.000 | 0.000 |
| 4 | 68 | MEFA | 28.080 | 11.169 | 0.229 | 0.090 | 1.000 | 0.400 | 0.600 | 0.000 | 0.000 | 0.000 | 0.000 |
| 5 | 76 | OXA | 20.360 | 9.827 | 0.246 | 0.119 | 1.000 | 0.000 | 1.000 | 0.000 | 0.000 | 0.000 | 0.000 |
| 6 | 92 | CATB | 15.920 | 8.093 | 0.249 | 0.127 | 1.000 | 0.520 | 0.480 | 0.000 | 0.000 | 0.000 | 0.000 |
| 7 | 162 | EREA | 31.360 | 13.187 | 0.254 | 0.106 | 1.000 | 0.080 | 0.920 | 0.000 | 0.000 | 0.000 | 0.000 |
| 8 | 174 | TEM | 25.720 | 9.965 | 0.306 | 0.119 | 1.000 | 0.000 | 1.000 | 0.000 | 0.000 | 0.000 | 0.000 |
| 9 | 193 | CML | 17.040 | 8.904 | 0.129 | 0.068 | 1.000 | 0.000 | 1.000 | 0.000 | 0.000 | 0.000 | 0.000 |
| 10 | 207 | NIMA | 13.560 | 7.235 | 0.292 | 0.154 | 1.000 | 1.000 | 0.000 | 0.000 | 0.000 | 0.000 | 0.000 |
| 11 | 239 | DHFR | 13.680 | 5.949 | 0.285 | 0.125 | 1.000 | 0.280 | 0.720 | 0.000 | 0.000 | 0.000 | 0.000 |
| 12 | 246 | FOLP | 28.680 | 10.664 | 0.240 | 0.090 | 1.000 | 1.000 | 0.000 | 0.000 | 0.000 | 0.000 | 0.000 |
| 13 | 274 | PARC | 36.040 | 11.374 | 0.162 | 0.051 | 1.000 | 0.400 | 0.600 | 0.000 | 0.000 | 0.000 | 0.000 |
| 14 | 276 | SHV | 24.760 | 11.399 | 0.283 | 0.130 | 1.000 | 0.040 | 0.960 | 0.000 | 0.000 | 0.000 | 0.000 |
| 15 | 284 | DFRA | 18.440 | 7.932 | 0.331 | 0.142 | 1.000 | 1.000 | 0.000 | 0.000 | 0.000 | 0.000 | 0.000 |
| 16 | 295 | SOXS | 15.120 | 4.494 | 0.456 | 0.135 | 1.000 | 1.000 | 0.000 | 0.000 | 0.000 | 0.000 | 0.000 |
| 17 | 321 | TSNR | 23.840 | 8.030 | 0.302 | 0.101 | 1.000 | 1.000 | 0.000 | 0.000 | 0.000 | 0.000 | 0.000 |
| 18 | 413 | OMPD | 26.440 | 9.739 | 0.244 | 0.090 | 1.000 | 1.000 | 0.000 | 0.000 | 0.000 | 0.000 | 0.000 |
| 19 | 486 | LMRA | 10.760 | 4.807 | 0.187 | 0.084 | 1.000 | 1.000 | 0.000 | 0.000 | 0.000 | 0.000 | 0.000 |
| 20 | 505 | CARB | 21.400 | 8.436 | 0.236 | 0.093 | 1.000 | 1.000 | 0.000 | 0.000 | 0.000 | 0.000 | 0.000 |
| 21 | 506 | ANT3- | 19.320 | 6.606 | 0.242 | 0.083 | 1.000 | 0.920 | 0.080 | 0.000 | 0.000 | 0.000 | 0.000 |
| | | DPRIME | | | | | | | | | | | |
| 22 | 555 | QNRS | 17.080 | 8.067 | 0.258 | 0.122 | 1.000 | 0.360 | 0.640 | 0.000 | 0.000 | 0.000 | 0.000 |
| 23 | 558 | VANWI | 20.640 | 9.591 | 0.183 | 0.085 | 1.000 | 1.000 | 0.000 | 0.000 | 0.000 | 0.000 | 0.000 |
| 24 | 578 | TETX | 30.360 | 13.391 | 0.259 | 0.115 | 1.000 | 0.280 | 0.720 | 0.000 | 0.000 | 0.000 | 0.000 |
| 25 | 652 | VGA | 38.120 | 14.001 | 0.242 | 0.089 | 1.000 | 0.880 | 0.120 | 0.000 | 0.000 | 0.000 | 0.000 |
| 26 | 682 | MOX | 42.960 | 14.226 | 0.372 | 0.123 | 1.000 | 0.400 | 0.600 | 0.000 | 0.000 | 0.000 | 0.000 |
| 27 | 694 | ACT | 41.480 | 18.136 | 0.330 | 0.145 | 1.000 | 0.120 | 0.880 | 0.000 | 0.000 | 0.000 | 0.000 |
| 28 | 717 | VANRE | 23.160 | 8.740 | 0.331 | 0.124 | 1.000 | 1.000 | 0.000 | 0.000 | 0.000 | 0.000 | 0.000 |
| 29 | 749 | RMTB | 8.320 | 3.859 | 0.315 | 0.151 | 1.000 | 1.000 | 0.000 | 0.000 | 0.000 | 0.000 | 0.000 |
| 30 | 778 | DHA | 23.760 | 11.921 | 0.208 | 0.104 | 1.000 | 0.000 | 1.000 | 0.000 | 0.000 | 0.000 | 0.000 |
| 31 | 789 | CMY | 31.160 | 16.570 | 0.271 | 0.144 | 1.000 | 0.000 | 1.000 | 0.000 | 0.000 | 0.000 | 0.000 |
| 32 | 797 | FACT | 25.440 | 16.269 | 0.151 | 0.096 | 1.000 | 1.000 | 0.000 | 0.000 | 0.000 | 0.000 | 0.000 |
| 33 | 819 | OKP | 30.840 | 15.763 | 0.355 | 0.181 | 1.000 | 0.040 | 0.960 | 0.000 | 0.000 | 0.000 | 0.000 |
| 34 | 973 | IMP | 20.240 | 10.849 | 0.270 | 0.146 | 1.000 | 0.200 | 0.800 | 0.000 | 0.000 | 0.000 | 0.000 |
| 35 | 1048 | VIM | 18.640 | 9.420 | 0.230 | 0.116 | 1.000 | 0.040 | 0.960 | 0.000 | 0.000 | 0.000 | 0.000 |
| 36 | 1135 | PBP1B | 34.640 | 13.997 | 0.140 | 0.056 | 1.000 | 1.000 | 0.000 | 0.000 | 0.000 | 0.000 | 0.000 |
| 37 | 1146 | MPHE | 23.000 | 10.794 | 0.259 | 0.122 | 1.000 | 0.400 | 0.600 | 0.000 | 0.000 | 0.000 | 0.000 |
| 38 | 1182 | VATE | 16.760 | 6.597 | 0.258 | 0.102 | 1.000 | 0.600 | 0.400 | 0.000 | 0.000 | 0.000 | 0.000 |
| 39 | 1214 | OPRJ | 49.200 | 25.987 | 0.339 | 0.179 | 1.000 | 1.000 | 0.000 | 0.000 | 0.000 | 0.000 | 0.000 |
| 40 | 1254 | FOSB | 14.600 | 5.370 | 0.343 | 0.124 | 1.000 | 1.000 | 0.000 | 0.000 | 0.000 | 0.000 | 0.000 |
| 41 | 1271 | VPH | 28.960 | 16.592 | 0.333 | 0.191 | 1.000 | 1.000 | 0.000 | 0.000 | 0.000 | 0.000 | 0.000 |
| 42 | 1283 | SULI | 26.160 | 11.912 | 0.309 | 0.140 | 1.000 | 0.120 | 0.880 | 0.000 | 0.000 | 0.000 | 0.000 |
| 43 | 1297 | TET40 | 19.960 | 8.801 | 0.162 | 0.071 | 1.000 | 0.480 | 0.520 | 0.000 | 0.000 | 0.000 | 0.000 |
| 44 | 1389 | CPXAR | 18.720 | 8.483 | 0.265 | 0.121 | 1.000 | 0.520 | 0.480 | 0.000 | 0.000 | 0.000 | 0.000 |
| 45 | 1392 | AAC6- | 11.440 | 5.973 | 0.204 | 0.106 | 1.000 | 0.720 | 0.280 | 0.000 | 0.000 | 0.000 | 0.000 |
| | | PRIME | | | | | | | | | | | |
| 46 | 1422 | VGBB | 32.960 | 10.737 | 0.367 | 0.120 | 1.000 | 1.000 | 0.000 | 0.000 | 0.000 | 0.000 | 0.000 |
| 47 | 1440 | FOSC | 16.400 | 8.201 | 0.293 | 0.146 | 1.000 | 1.000 | 0.000 | 0.000 | 0.000 | 0.000 | 0.000 |
| 48 | 1535 | LNUA | 12.160 | 6.743 | 0.249 | 0.138 | 1.000 | 0.520 | 0.480 | 0.000 | 0.000 | 0.000 | 0.000 |
| 49 | 1569 | PARE | 50.680 | 20.954 | 0.268 | 0.111 | 1.000 | 0.680 | 0.320 | 0.000 | 0.000 | 0.000 | 0.000 |
| 50 | 1695 | NDM | 17.960 | 10.990 | 0.218 | 0.133 | 1.000 | 0.960 | 0.040 | 0.000 | 0.000 | 0.000 | 0.000 |
| 51 | 1702 | SPG | 23.480 | 14.726 | 0.273 | 0.171 | 1.000 | 1.000 | 0.000 | 0.000 | 0.000 | 0.000 | 0.000 |
| 52 | 1753 | VEB | 26.640 | 14.059 | 0.294 | 0.155 | 1.000 | 0.120 | 0.880 | 0.000 | 0.000 | 0.000 | 0.000 |
| 53 | 1953 | LNUB | 24.000 | 9.069 | 0.296 | 0.112 | 1.000 | 0.840 | 0.160 | 0.000 | 0.000 | 0.000 | 0.000 |
| 54 | 2026 | ERMA | 21.160 | 10.172 | 0.286 | 0.137 | 1.000 | 0.920 | 0.080 | 0.000 | 0.000 | 0.000 | 0.000 |
| 55 | 2357 | SULII | 27.960 | 8.763 | 0.342 | 0.108 | 1.000 | 0.200 | 0.800 | 0.000 | 0.000 | 0.000 | 0.000 |
| 56 | 2517 | TET37 | 11.040 | 4.335 | 0.332 | 0.133 | 1.000 | 1.000 | 0.000 | 0.000 | 0.000 | 0.000 | 0.000 |
| 57 | 2822 | EMRK | 30.440 | 11.285 | 0.287 | 0.106 | 1.000 | 1.000 | 0.000 | 0.000 | 0.000 | 0.000 | 0.000 |
| 58 | 2999 | MPHB | 26.960 | 11.156 | 0.295 | 0.123 | 1.000 | 1.000 | 0.000 | 0.000 | 0.000 | 0.000 | 0.000 |
| 59 | 3024 | VANYM | 25.000 | 8.401 | 0.351 | 0.118 | 1.000 | 1.000 | 0.000 | 0.000 | 0.000 | 0.000 | 0.000 |
| 60 | 3041 | MECC | 67.560 | 60.175 | 0.336 | 0.299 | 1.000 | 1.000 | 0.000 | 0.000 | 0.000 | 0.000 | 0.000 |
| 61 | 3128 | TUFAB | 30.400 | 14.543 | 0.255 | 0.122 | 1.000 | 0.680 | 0.320 | 0.000 | 0.000 | 0.000 | 0.000 |
| 62 | 3176 | AMRB | 67.960 | 30.847 | 0.216 | 0.098 | 1.000 | 1.000 | 0.000 | 0.000 | 0.000 | 0.000 | 0.000 |
| 63 | 3270 | IRI | 53.080 | 22.156 | 0.366 | 0.153 | 1.000 | 1.000 | 0.000 | 0.000 | 0.000 | 0.000 | 0.000 |
| 64 | 3314 | RPOB | 40.120 | 14.661 | 0.113 | 0.041 | 1.000 | 0.000 | 1.000 | 0.000 | 0.000 | 0.000 | 0.000 |
| 65 | 3332 | TET35 | 19.920 | 10.352 | 0.178 | 0.093 | 1.000 | 1.000 | 0.000 | 0.000 | 0.000 | 0.000 | 0.000 |
| 66 | 3370 | CFRA | 35.000 | 13.994 | 0.331 | 0.133 | 1.000 | 1.000 | 0.000 | 0.000 | 0.000 | 0.000 | 0.000 |
| 67 | 3513 | BRP | 10.920 | 3.121 | 0.270 | 0.078 | 1.000 | 1.000 | 0.000 | 0.000 | 0.000 | 0.000 | 0.000 |
| 68 | 3613 | APH3- | 17.640 | 8.602 | 0.218 | 0.105 | 1.000 | 1.000 | 0.000 | 0.000 | 0.000 | 0.000 | 0.000 |
| | | PRIME | | | | | | | | | | | |
| 69 | 3697 | TETM | 55.200 | 26.608 | 0.286 | 0.138 | 1.000 | 0.480 | 0.520 | 0.000 | 0.000 | 0.000 | 0.000 |
| 70 | 3778 | IND | 23.840 | 7.548 | 0.322 | 0.102 | 1.000 | 0.040 | 0.960 | 0.000 | 0.000 | 0.000 | 0.000 |

Simulation settings:
k-mers: ‘constant’, k = 10
Gene coverage: 1
Number of genes: 1
Errors: ‘off’
Penalty score: 0.1
Thresholding and entropy screening was deactivated

TABLE S3
Resistance genes simulations with thresholding and entropy screening

| | | | Avg | StDev | Avg | StDev | | Identification level | | | | | |
|---|---|---|---|---|---|---|---|---|---|---|---|---|---|
| | Data- | Gene | block for | block for | coverage for | coverage for | | (fraction of trials) | | | | Avg | StDev |
| | base | sub- | identifi- | identifi- | identifi- | identifi- | Accu- | Specific | Sub- | | Incor- | false | false |
| No. | No. | class | cation | cation | cation | cation | racy | gene | class | Class | rect | positives | positives |
| 1 | 4 | VANZA | 18.960 | 9.977 | 0.385 | 0.203 | 1.000 | 1.000 | 0.000 | 0.000 | 0.000 | 0.000 | 0.000 |
| 2 | 52 | VANWG | 15.600 | 7.444 | 0.183 | 0.087 | 1.000 | 0.400 | 0.600 | 0.000 | 0.000 | 0.000 | 0.000 |
| 3 | 62 | CTX | 19.080 | 14.003 | 0.217 | 0.158 | 1.000 | 0.000 | 1.000 | 0.000 | 0.000 | 0.000 | 0.000 |
| 4 | 68 | MEFA | 30.600 | 14.471 | 0.249 | 0.119 | 1.000 | 0.520 | 0.480 | 0.000 | 0.000 | 0.000 | 0.000 |
| 5 | 76 | OXA | 14.600 | 7.599 | 0.175 | 0.091 | 1.000 | 0.000 | 1.000 | 0.000 | 0.000 | 0.000 | 0.000 |
| 6 | 92 | CATB | 14.880 | 6.431 | 0.229 | 0.099 | 1.000 | 0.440 | 0.560 | 0.000 | 0.000 | 0.000 | 0.000 |
| 7 | 162 | EREA | 18.680 | 8.513 | 0.150 | 0.069 | 1.000 | 0.240 | 0.760 | 0.000 | 0.000 | 0.000 | 0.000 |
| 8 | 174 | TEM | 17.120 | 4.927 | 0.202 | 0.059 | 1.000 | 0.000 | 1.000 | 0.000 | 0.000 | 0.000 | 0.000 |
| 9 | 193 | CML | 17.560 | 9.070 | 0.133 | 0.068 | 1.000 | 0.240 | 0.760 | 0.000 | 0.000 | 0.000 | 0.000 |
| 10 | 207 | NIMA | 9.520 | 4.144 | 0.204 | 0.091 | 1.000 | 1.000 | 0.000 | 0.000 | 0.000 | 0.000 | 0.000 |
| 11 | 239 | DHFR | 12.200 | 5.824 | 0.252 | 0.119 | 1.000 | 0.160 | 0.840 | 0.000 | 0.000 | 0.000 | 0.000 |
| 12 | 246 | FOLP | 30.800 | 21.221 | 0.257 | 0.179 | 1.000 | 1.000 | 0.000 | 0.000 | 0.000 | 0.000 | 0.000 |
| 13 | 274 | PARC | 59.480 | 30.040 | 0.266 | 0.134 | 1.000 | 0.280 | 0.720 | 0.000 | 0.000 | 0.000 | 0.000 |
| 14 | 276 | SHV | 19.040 | 9.158 | 0.216 | 0.105 | 1.000 | 0.120 | 0.880 | 0.000 | 0.000 | 0.000 | 0.000 |
| 15 | 284 | DFRA | 14.960 | 5.504 | 0.265 | 0.099 | 1.000 | 1.000 | 0.000 | 0.000 | 0.000 | 0.000 | 0.000 |
| 16 | 295 | SOXS | 9.560 | 2.501 | 0.283 | 0.075 | 1.000 | 1.000 | 0.000 | 0.000 | 0.000 | 0.000 | 0.000 |
| 17 | 321 | TSNR | 23.560 | 15.202 | 0.296 | 0.192 | 1.000 | 1.000 | 0.000 | 0.000 | 0.000 | 0.000 | 0.000 |
| 18 | 413 | OMPD | 26.720 | 10.073 | 0.246 | 0.093 | 1.000 | 1.000 | 0.000 | 0.000 | 0.000 | 0.000 | 0.000 |
| 19 | 486 | LMRA | 9.320 | 3.648 | 0.160 | 0.062 | 1.000 | 1.000 | 0.000 | 0.000 | 0.000 | 0.000 | 0.000 |
| 20 | 505 | CARB | 21.760 | 8.171 | 0.238 | 0.089 | 1.000 | 1.000 | 0.000 | 0.000 | 0.000 | 0.000 | 0.000 |
| 21 | 506 | ANT3- | 20.200 | 5.701 | 0.247 | 0.072 | 1.000 | 1.000 | 0.000 | 0.000 | 0.000 | 0.000 | 0.000 |
| | | DPRIME | | | | | | | | | | | |
| 22 | 555 | QNRS | 23.160 | 6.656 | 0.347 | 0.101 | 1.000 | 0.400 | 0.600 | 0.000 | 0.000 | 0.000 | 0.000 |
| 23 | 558 | VANWI | 16.480 | 5.363 | 0.145 | 0.047 | 1.000 | 1.000 | 0.000 | 0.000 | 0.000 | 0.000 | 0.000 |
| 24 | 578 | TETX | 28.840 | 17.112 | 0.246 | 0.146 | 1.000 | 0.560 | 0.440 | 0.000 | 0.000 | 0.000 | 0.000 |
| 25 | 652 | VGA | 36.120 | 19.633 | 0.229 | 0.124 | 1.000 | 0.880 | 0.120 | 0.000 | 0.000 | 0.000 | 0.000 |
| 26 | 682 | MOX | 35.440 | 16.971 | 0.305 | 0.146 | 1.000 | 0.040 | 0.960 | 0.000 | 0.000 | 0.000 | 0.000 |
| 27 | 694 | ACT | 35.280 | 13.430 | 0.279 | 0.106 | 1.000 | 0.520 | 0.480 | 0.000 | 0.000 | 0.000 | 0.000 |
| 28 | 717 | VANRE | 23.240 | 9.212 | 0.329 | 0.131 | 1.000 | 1.000 | 0.000 | 0.000 | 0.000 | 0.000 | 0.000 |
| 29 | 749 | RMTB | 4.720 | 2.542 | 0.173 | 0.098 | 1.000 | 1.000 | 0.000 | 0.000 | 0.000 | 0.000 | 0.000 |
| 30 | 778 | DHA | 15.600 | 5.431 | 0.136 | 0.048 | 1.000 | 0.000 | 1.000 | 0.000 | 0.000 | 0.000 | 0.000 |
| 31 | 789 | CMY | 24.880 | 13.618 | 0.216 | 0.118 | 1.000 | 0.000 | 1.000 | 0.000 | 0.000 | 0.000 | 0.000 |
| 32 | 797 | FACT | 24.800 | 18.241 | 0.146 | 0.108 | 1.000 | 1.000 | 0.000 | 0.000 | 0.000 | 0.000 | 0.000 |
| 33 | 819 | OKP | 26.920 | 11.849 | 0.307 | 0.135 | 1.000 | 0.080 | 0.920 | 0.000 | 0.000 | 0.000 | 0.000 |
| 34 | 973 | IMP | 14.920 | 8.441 | 0.198 | 0.112 | 1.000 | 0.240 | 0.760 | 0.000 | 0.000 | 0.000 | 0.000 |
| 35 | 1048 | VIM | 12.320 | 4.964 | 0.149 | 0.060 | 1.000 | 0.200 | 0.800 | 0.000 | 0.000 | 0.000 | 0.000 |
| 36 | 1135 | PBP1B | 29.440 | 11.832 | 0.119 | 0.048 | 1.000 | 1.000 | 0.000 | 0.000 | 0.000 | 0.000 | 0.000 |
| 37 | 1146 | MPHE | 22.760 | 9.858 | 0.255 | 0.110 | 1.000 | 0.200 | 0.800 | 0.000 | 0.000 | 0.000 | 0.000 |
| 38 | 1182 | VATE | 16.240 | 6.139 | 0.248 | 0.093 | 1.000 | 0.640 | 0.360 | 0.000 | 0.000 | 0.000 | 0.000 |
| 39 | 1214 | OPRJ | 56.720 | 23.183 | 0.389 | 0.160 | 1.000 | 1.000 | 0.000 | 0.000 | 0.000 | 0.000 | 0.000 |
| 40 | 1254 | FOSB | 22.720 | 9.222 | 0.527 | 0.216 | 1.000 | 0.960 | 0.040 | 0.000 | 0.000 | 0.000 | 0.000 |
| 41 | 1271 | VPH | 17.040 | 10.382 | 0.196 | 0.119 | 1.000 | 1.000 | 0.000 | 0.000 | 0.000 | 0.000 | 0.000 |
| 42 | 1283 | SULI | 29.040 | 14.607 | 0.339 | 0.173 | 1.000 | 0.080 | 0.920 | 0.000 | 0.000 | 0.000 | 0.000 |
| 43 | 1297 | TET40 | 10.240 | 3.666 | 0.083 | 0.030 | 1.000 | 0.360 | 0.640 | 0.000 | 0.000 | 0.000 | 0.000 |
| 44 | 1389 | CPXAR | 17.680 | 3.891 | 0.248 | 0.052 | 1.000 | 0.680 | 0.320 | 0.000 | 0.000 | 0.000 | 0.000 |
| 45 | 1392 | AAC6- | 6.720 | 3.736 | 0.119 | 0.066 | 1.000 | 0.760 | 0.240 | 0.000 | 0.000 | 0.000 | 0.000 |
| | | PRIME | | | | | | | | | | | |
| 46 | 1422 | VGBB | 37.960 | 11.043 | 0.420 | 0.122 | 1.000 | 1.000 | 0.000 | 0.000 | 0.000 | 0.000 | 0.000 |
| 47 | 1440 | FOSC | 13.200 | 5.164 | 0.233 | 0.089 | 1.000 | 1.000 | 0.000 | 0.000 | 0.000 | 0.000 | 0.000 |
| 48 | 1535 | LNUA | 8.360 | 5.195 | 0.171 | 0.105 | 1.000 | 0.480 | 0.520 | 0.000 | 0.000 | 0.000 | 0.000 |
| 49 | 1569 | PARE | 38.560 | 15.565 | 0.202 | 0.082 | 1.000 | 0.920 | 0.080 | 0.000 | 0.000 | 0.000 | 0.000 |
| 50 | 1695 | NDM | 19.960 | 14.519 | 0.241 | 0.176 | 1.000 | 1.000 | 0.000 | 0.000 | 0.000 | 0.000 | 0.000 |
| 51 | 1702 | SPG | 17.680 | 10.213 | 0.205 | 0.118 | 1.000 | 1.000 | 0.000 | 0.000 | 0.000 | 0.000 | 0.000 |
| 52 | 1753 | VEB | 33.480 | 21.219 | 0.369 | 0.236 | 1.000 | 0.040 | 0.960 | 0.000 | 0.000 | 0.000 | 0.000 |
| 53 | 1953 | LNUB | 28.000 | 9.305 | 0.344 | 0.115 | 1.000 | 0.920 | 0.080 | 0.000 | 0.000 | 0.000 | 0.000 |
| 54 | 2026 | ERMA | 23.600 | 12.832 | 0.317 | 0.172 | 1.000 | 0.920 | 0.080 | 0.000 | 0.000 | 0.000 | 0.000 |
| 55 | 2357 | SULII | 16.120 | 7.918 | 0.196 | 0.096 | 1.000 | 0.080 | 0.920 | 0.000 | 0.000 | 0.000 | 0.000 |
| 56 | 2517 | TET37 | 11.640 | 5.057 | 0.348 | 0.155 | 1.000 | 1.000 | 0.000 | 0.000 | 0.000 | 0.000 | 0.000 |
| 57 | 2822 | EMRK | 53.120 | 11.591 | 0.499 | 0.110 | 1.000 | 1.000 | 0.000 | 0.000 | 0.000 | 0.000 | 0.000 |
| 58 | 2999 | MPHB | 37.360 | 13.853 | 0.407 | 0.153 | 1.000 | 1.000 | 0.000 | 0.000 | 0.000 | 0.000 | 0.000 |
| 59 | 3024 | VANYM | 24.200 | 8.005 | 0.338 | 0.113 | 1.000 | 1.000 | 0.000 | 0.000 | 0.000 | 0.000 | 0.000 |
| 60 | 3041 | MECC | 75.400 | 59.431 | 0.374 | 0.295 | 1.000 | 1.000 | 0.000 | 0.000 | 0.000 | 0.000 | 0.000 |
| 61 | 3128 | TUFAB | 22.280 | 9.689 | 0.186 | 0.081 | 1.000 | 0.840 | 0.160 | 0.000 | 0.000 | 0.000 | 0.000 |
| 62 | 3176 | AMRB | 57.400 | 25.120 | 0.182 | 0.079 | 1.000 | 1.000 | 0.000 | 0.000 | 0.000 | 0.000 | 0.000 |
| 63 | 3270 | IRI | 79.120 | 12.640 | 0.543 | 0.087 | 1.000 | 1.000 | 0.000 | 0.000 | 0.000 | 0.000 | 0.000 |
| 64 | 3314 | RPOB | 53.360 | 17.411 | 0.149 | 0.049 | 1.000 | 0.000 | 1.000 | 0.000 | 0.000 | 0.000 | 0.000 |
| 65 | 3332 | TET35 | 20.240 | 10.948 | 0.180 | 0.096 | 1.000 | 1.000 | 0.000 | 0.000 | 0.000 | 0.000 | 0.000 |
| 66 | 3370 | CFRA | 34.840 | 15.407 | 0.327 | 0.146 | 1.000 | 1.000 | 0.000 | 0.000 | 0.000 | 0.000 | 0.000 |
| 67 | 3513 | BRP | 8.720 | 3.398 | 0.213 | 0.083 | 1.000 | 1.000 | 0.000 | 0.000 | 0.000 | 0.000 | 0.000 |
| 68 | 3613 | APH3- | 30.560 | 16.000 | 0.375 | 0.195 | 1.000 | 1.000 | 0.000 | 0.000 | 0.000 | 0.000 | 0.000 |
| | | PRIME | | | | | | | | | | | |
| 69 | 3697 | TETM | 46.840 | 19.433 | 0.243 | 0.100 | 1.000 | 0.560 | 0.440 | 0.000 | 0.000 | 0.000 | 0.000 |
| 70 | 3778 | IND | 17.000 | 5.715 | 0.229 | 0.075 | 1.000 | 0.160 | 0.840 | 0.000 | 0.000 | 0.000 | 0.000 |

Simulation settings:
k-mers: ‘constant’, k = 10
Gene coverage: 1
Number of genes: 1
Errors: ‘off’
Penalty score: 0.1
Thresholding: multiplier = 1, all factors (1-on)
Entropy screening: ‘rand’

TABLE S4
Resistance genes simulations with 8-mer blocks

| | | | Avg | StDev | Avg | StDev | | Identification level | | | | | |
|---|---|---|---|---|---|---|---|---|---|---|---|---|---|
| | Data- | Gene | block for | block for | coverage for | coverage for | | (fraction of trials) | | | | Avg | StDev |
| | base | sub- | identifi- | identifi- | identifi- | identifi- | Accu- | Specific | Sub- | | Incor- | false | false |
| No. | No. | class | cation | cation | cation | cation | racy | gene | class | Class | rect | positives | positives |
| 1 | 4 | VANZA | 20.360 | 10.327 | 0.331 | 0.170 | 1.000 | 1.000 | 0.000 | 0.000 | 0.000 | 0.000 | 0.000 |
| 2 | 52 | VANWG | 37.800 | 17.963 | 0.356 | 0.170 | 1.000 | 0.440 | 0.560 | 0.000 | 0.000 | 0.000 | 0.000 |
| 3 | 62 | CTX | 37.280 | 13.252 | 0.339 | 0.120 | 1.000 | 0.000 | 1.000 | 0.000 | 0.000 | 0.000 | 0.000 |
| 4 | 68 | MEFA | 43.960 | 19.385 | 0.288 | 0.127 | 1.000 | 0.520 | 0.480 | 0.000 | 0.000 | 0.000 | 0.000 |
| 5 | 76 | OXA | 35.640 | 18.259 | 0.343 | 0.176 | 1.000 | 0.000 | 1.000 | 0.000 | 0.000 | 0.000 | 0.000 |
| 6 | 92 | CATB | 22.160 | 9.711 | 0.278 | 0.122 | 1.000 | 0.320 | 0.680 | 0.000 | 0.000 | 0.000 | 0.000 |
| 7 | 162 | EREA | 38.720 | 17.387 | 0.251 | 0.113 | 1.000 | 0.120 | 0.880 | 0.000 | 0.000 | 0.000 | 0.000 |
| 8 | 174 | TEM | 37.640 | 13.778 | 0.359 | 0.131 | 1.000 | 0.000 | 1.000 | 0.000 | 0.000 | 0.000 | 0.000 |
| 9 | 193 | CML | 26.160 | 15.032 | 0.159 | 0.091 | 1.000 | 0.000 | 1.000 | 0.000 | 0.000 | 0.000 | 0.000 |
| 10 | 207 | NIMA | 13.800 | 5.951 | 0.236 | 0.102 | 1.000 | 0.960 | 0.040 | 0.000 | 0.000 | 0.000 | 0.000 |
| 11 | 239 | DHFR | 23.680 | 9.040 | 0.393 | 0.151 | 1.000 | 0.320 | 0.680 | 0.000 | 0.000 | 0.000 | 0.000 |
| 12 | 246 | FOLP | 40.833 | 19.699 | 0.274 | 0.132 | 0.960 | 0.920 | 0.040 | 0.000 | 0.040 | 0.000 | 0.000 |
| 13 | 274 | PARC | 72.720 | 31.383 | 0.262 | 0.113 | 1.000 | 0.280 | 0.720 | 0.000 | 0.000 | 0.000 | 0.000 |
| 14 | 276 | SHV | 32.400 | 14.939 | 0.297 | 0.137 | 1.000 | 0.000 | 1.000 | 0.000 | 0.000 | 0.000 | 0.000 |
| 15 | 284 | DFRA | 29.080 | 9.639 | 0.416 | 0.137 | 1.000 | 1.000 | 0.000 | 0.000 | 0.000 | 0.000 | 0.000 |
| 16 | 295 | SOXS | 18.320 | 8.764 | 0.446 | 0.215 | 1.000 | 1.000 | 0.000 | 0.000 | 0.000 | 0.000 | 0.000 |
| 17 | 321 | TSNR | 38.636 | 20.254 | 0.391 | 0.206 | 0.880 | 0.880 | 0.000 | 0.000 | 0.120 | 0.000 | 0.000 |
| 18 | 413 | OMPD | 37.560 | 12.203 | 0.279 | 0.090 | 1.000 | 1.000 | 0.000 | 0.000 | 0.000 | 0.000 | 0.000 |
| 19 | 486 | LMRA | 16.800 | 7.130 | 0.234 | 0.098 | 1.000 | 1.000 | 0.000 | 0.000 | 0.000 | 0.000 | 0.000 |
| 20 | 505 | CARB | 31.200 | 11.576 | 0.276 | 0.104 | 1.000 | 1.000 | 0.000 | 0.000 | 0.000 | 0.000 | 0.000 |
| 21 | 506 | ANT3- | 27.480 | 9.896 | 0.275 | 0.099 | 1.000 | 0.960 | 0.040 | 0.000 | 0.000 | 0.000 | 0.000 |
| | | DPRIME | | | | | | | | | | | |
| 22 | 555 | QNRS | 28.680 | 11.821 | 0.345 | 0.143 | 1.000 | 0.360 | 0.640 | 0.000 | 0.000 | 0.000 | 0.000 |
| 23 | 558 | VANWI | 32.280 | 15.038 | 0.229 | 0.107 | 1.000 | 1.000 | 0.000 | 0.000 | 0.000 | 0.000 | 0.000 |
| 24 | 578 | TETX | 48.560 | 14.463 | 0.332 | 0.099 | 1.000 | 0.240 | 0.760 | 0.000 | 0.000 | 0.000 | 0.000 |
| 25 | 652 | VGA | 61.739 | 32.433 | 0.312 | 0.164 | 0.920 | 0.840 | 0.080 | 0.000 | 0.080 | 0.000 | 0.000 |
| 26 | 682 | MOX | 55.400 | 20.145 | 0.384 | 0.140 | 1.000 | 0.320 | 0.680 | 0.000 | 0.000 | 0.000 | 0.000 |
| 27 | 694 | ACT | 43.320 | 22.090 | 0.277 | 0.141 | 1.000 | 0.080 | 0.920 | 0.000 | 0.000 | 0.000 | 0.000 |
| 28 | 717 | VANRE | 31.400 | 17.772 | 0.361 | 0.205 | 1.000 | 1.000 | 0.000 | 0.000 | 0.000 | 0.000 | 0.000 |
| 29 | 749 | RMTB | 11.040 | 5.111 | 0.344 | 0.161 | 1.000 | 1.000 | 0.000 | 0.000 | 0.000 | 0.000 | 0.000 |
| 30 | 778 | DHA | 41.920 | 20.004 | 0.293 | 0.140 | 1.000 | 0.040 | 0.960 | 0.000 | 0.000 | 0.000 | 0.000 |
| 31 | 789 | CMY | 39.640 | 16.153 | 0.275 | 0.112 | 1.000 | 0.000 | 1.000 | 0.000 | 0.000 | 0.000 | 0.000 |
| 32 | 797 | FACT | 40.167 | 25.862 | 0.190 | 0.122 | 0.960 | 0.960 | 0.000 | 0.000 | 0.040 | 0.000 | 0.000 |
| 33 | 819 | OKP | 45.760 | 22.244 | 0.424 | 0.206 | 1.000 | 0.040 | 0.960 | 0.000 | 0.000 | 0.000 | 0.000 |
| 34 | 973 | IMP | 31.520 | 12.210 | 0.339 | 0.131 | 1.000 | 0.040 | 0.960 | 0.000 | 0.000 | 0.000 | 0.000 |
| 35 | 1048 | VIM | 27.920 | 14.250 | 0.277 | 0.142 | 1.000 | 0.080 | 0.920 | 0.000 | 0.000 | 0.000 | 0.000 |
| 36 | 1135 | PBP1B | 46.520 | 22.006 | 0.151 | 0.071 | 1.000 | 1.000 | 0.000 | 0.000 | 0.000 | 0.000 | 0.000 |
| 37 | 1146 | MPHE | 43.720 | 17.119 | 0.394 | 0.154 | 1.000 | 0.280 | 0.720 | 0.000 | 0.000 | 0.000 | 0.000 |
| 38 | 1182 | VATE | 20.800 | 8.818 | 0.257 | 0.109 | 1.000 | 0.200 | 0.800 | 0.000 | 0.000 | 0.000 | 0.000 |
| 39 | 1214 | OPRJ | 69.320 | 31.201 | 0.383 | 0.172 | 1.000 | 1.000 | 0.000 | 0.000 | 0.000 | 0.000 | 0.000 |
| 40 | 1254 | FOSB | 17.880 | 7.839 | 0.337 | 0.148 | 1.000 | 0.920 | 0.080 | 0.000 | 0.000 | 0.000 | 0.000 |
| 41 | 1271 | VPH | 30.000 | 16.427 | 0.275 | 0.150 | 1.000 | 1.000 | 0.000 | 0.000 | 0.000 | 0.000 | 0.000 |
| 42 | 1283 | SULI | 45.080 | 17.949 | 0.426 | 0.170 | 1.000 | 0.280 | 0.720 | 0.000 | 0.000 | 0.000 | 0.000 |
| 43 | 1297 | TET40 | 34.080 | 18.841 | 0.223 | 0.123 | 1.000 | 0.560 | 0.440 | 0.000 | 0.000 | 0.000 | 0.000 |
| 44 | 1389 | CPXAR | 27.000 | 8.211 | 0.306 | 0.093 | 1.000 | 0.360 | 0.640 | 0.000 | 0.000 | 0.000 | 0.000 |
| 45 | 1392 | AAC6- | 17.160 | 9.168 | 0.245 | 0.130 | 1.000 | 0.640 | 0.360 | 0.000 | 0.000 | 0.000 | 0.000 |
| | | PRIME | | | | | | | | | | | |
| 46 | 1422 | VGBB | 39.560 | 16.153 | 0.354 | 0.144 | 1.000 | 1.000 | 0.000 | 0.000 | 0.000 | 0.000 | 0.000 |
| 47 | 1440 | FOSC | 17.360 | 9.691 | 0.252 | 0.140 | 1.000 | 1.000 | 0.000 | 0.000 | 0.000 | 0.000 | 0.000 |
| 48 | 1535 | LNUA | 15.280 | 8.975 | 0.250 | 0.148 | 1.000 | 0.400 | 0.600 | 0.000 | 0.000 | 0.000 | 0.000 |
| 49 | 1569 | PARE | 72.800 | 23.189 | 0.308 | 0.098 | 1.000 | 0.320 | 0.680 | 0.000 | 0.000 | 0.000 | 0.000 |
| 50 | 1695 | NDM | 25.280 | 9.410 | 0.248 | 0.092 | 1.000 | 1.000 | 0.000 | 0.000 | 0.000 | 0.000 | 0.000 |
| 51 | 1702 | SPG | 29.480 | 13.574 | 0.273 | 0.125 | 1.000 | 1.000 | 0.000 | 0.000 | 0.000 | 0.000 | 0.000 |
| 52 | 1753 | VEB | 33.280 | 15.568 | 0.295 | 0.138 | 1.000 | 0.000 | 1.000 | 0.000 | 0.000 | 0.000 | 0.000 |
| 53 | 1953 | LNUB | 36.480 | 13.226 | 0.362 | 0.131 | 1.000 | 0.560 | 0.440 | 0.000 | 0.000 | 0.000 | 0.000 |
| 54 | 2026 | ERMA | 31.000 | 14.018 | 0.337 | 0.152 | 1.000 | 0.720 | 0.280 | 0.000 | 0.000 | 0.000 | 0.000 |
| 55 | 2357 | SULII | 30.417 | 13.445 | 0.296 | 0.131 | 0.960 | 0.000 | 0.960 | 0.000 | 0.040 | 0.000 | 0.000 |
| 56 | 2517 | TET37 | 17.440 | 6.868 | 0.415 | 0.165 | 1.000 | 1.000 | 0.000 | 0.000 | 0.000 | 0.000 | 0.000 |
| 57 | 2822 | EMRK | 41.160 | 19.686 | 0.310 | 0.148 | 1.000 | 1.000 | 0.000 | 0.000 | 0.000 | 0.000 | 0.000 |
| 58 | 2999 | MPHB | 29.667 | 14.577 | 0.260 | 0.128 | 0.960 | 0.960 | 0.000 | 0.000 | 0.040 | 0.000 | 0.000 |
| 59 | 3024 | VANYM | 46.680 | 19.991 | 0.528 | 0.224 | 1.000 | 0.960 | 0.040 | 0.000 | 0.000 | 0.160 | 0.374 |
| 60 | 3041 | MECC | 214.667 | 68.542 | 0.853 | 0.272 | 0.960 | 0.280 | 0.680 | 0.000 | 0.040 | 0.000 | 0.000 |
| 61 | 3128 | TUFAB | 33.760 | 14.042 | 0.226 | 0.095 | 1.000 | 0.400 | 0.600 | 0.000 | 0.000 | 0.000 | 0.000 |
| 62 | 3176 | AMRB | 97.320 | 39.918 | 0.248 | 0.102 | 1.000 | 1.000 | 0.000 | 0.000 | 0.000 | 0.000 | 0.000 |
| 63 | 3270 | IRI | 62.125 | 22.462 | 0.344 | 0.125 | 0.960 | 0.960 | 0.000 | 0.000 | 0.040 | 0.000 | 0.000 |
| 64 | 3314 | RPOB | 89.320 | 57.045 | 0.201 | 0.128 | 1.000 | 0.160 | 0.840 | 0.000 | 0.000 | 0.000 | 0.000 |
| 65 | 3332 | TET35 | 22.240 | 11.598 | 0.159 | 0.083 | 1.000 | 1.000 | 0.000 | 0.000 | 0.000 | 0.000 | 0.000 |
| 66 | 3370 | CFRA | 70.960 | 19.659 | 0.537 | 0.149 | 1.000 | 1.000 | 0.000 | 0.000 | 0.000 | 0.000 | 0.000 |
| 67 | 3513 | BRP | 12.320 | 6.549 | 0.242 | 0.127 | 1.000 | 1.000 | 0.000 | 0.000 | 0.000 | 0.000 | 0.000 |
| 68 | 3613 | APH3- | 40.120 | 17.050 | 0.397 | 0.169 | 1.000 | 1.000 | 0.000 | 0.000 | 0.000 | 0.000 | 0.000 |
| | | PRIME | | | | | | | | | | | |
| 69 | 3697 | TETM | 77.200 | 37.236 | 0.320 | 0.155 | 1.000 | 0.400 | 0.600 | 0.000 | 0.000 | 0.000 | 0.000 |
| 70 | 3778 | IND | 36.920 | 12.124 | 0.402 | 0.132 | 1.000 | 0.120 | 0.880 | 0.000 | 0.000 | 0.000 | 0.000 |

Simulation settings:
k-mers: ‘constant’, k = 8
Gene coverage: 1
Number of genes: 1
Errors: ‘off’
Penalty score: 0.1
Thresholding: multiplier = 1, all factors (1-on)
Entropy screening: ‘rand’

TABLE S5
Resistance genes simulations with 12-mer blocks

| | | | Avg | StDev | Avg | StDev | | Identification level | | | | | |
|---|---|---|---|---|---|---|---|---|---|---|---|---|---|
| | Data- | Gene | block for | block for | coverage for | coverage for | | (fraction of trials) | | | | Avg | StDev |
| | base | sub- | identifi- | identifi- | identifi- | identifi- | Accu- | Specific | Sub- | | Incor- | false | false |
| No. | No. | class | cation | cation | cation | cation | racy | gene | class | Class | rect | positives | positives |
| 1 | 4 | VANZA | 13.080 | 3.818 | 0.312 | 0.094 | 1.000 | 1.000 | 0.000 | 0.000 | 0.000 | 0.000 | 0.000 |
| 2 | 52 | VANWG | 8.440 | 2.987 | 0.115 | 0.042 | 1.000 | 0.720 | 0.280 | 0.000 | 0.000 | 0.000 | 0.000 |
| 3 | 62 | CTX | 11.640 | 6.915 | 0.150 | 0.094 | 1.000 | 0.000 | 1.000 | 0.000 | 0.000 | 0.000 | 0.000 |
| 4 | 68 | MEFA | 12.640 | 4.212 | 0.121 | 0.041 | 1.000 | 0.480 | 0.520 | 0.000 | 0.000 | 0.000 | 0.000 |
| 5 | 76 | OXA | 7.200 | 4.203 | 0.098 | 0.058 | 1.000 | 0.000 | 1.000 | 0.000 | 0.000 | 0.000 | 0.000 |
| 6 | 92 | CATB | 6.520 | 2.756 | 0.113 | 0.050 | 1.000 | 0.080 | 0.920 | 0.000 | 0.000 | 0.000 | 0.000 |
| 7 | 162 | EREA | 5.200 | 2.021 | 0.046 | 0.019 | 1.000 | 0.440 | 0.560 | 0.000 | 0.000 | 0.000 | 0.000 |
| 8 | 174 | TEM | 11.640 | 6.415 | 0.159 | 0.092 | 1.000 | 0.000 | 1.000 | 0.000 | 0.000 | 0.000 | 0.000 |
| 9 | 193 | CML | 5.520 | 4.360 | 0.047 | 0.039 | 1.000 | 0.000 | 1.000 | 0.000 | 0.000 | 0.000 | 0.000 |
| 10 | 207 | NIMA | 8.600 | 5.845 | 0.203 | 0.153 | 1.000 | 1.000 | 0.000 | 0.000 | 0.000 | 0.000 | 0.000 |
| 11 | 239 | DHFR | 10.800 | 4.330 | 0.262 | 0.109 | 1.000 | 0.280 | 0.720 | 0.000 | 0.000 | 0.000 | 0.000 |
| 12 | 246 | FOLP | 14.160 | 5.843 | 0.137 | 0.056 | 1.000 | 1.000 | 0.000 | 0.000 | 0.000 | 0.000 | 0.000 |
| 13 | 274 | PARC | 20.400 | 7.539 | 0.106 | 0.039 | 1.000 | 0.560 | 0.440 | 0.000 | 0.000 | 0.000 | 0.000 |
| 14 | 276 | SHV | 11.800 | 9.000 | 0.157 | 0.123 | 1.000 | 0.000 | 1.000 | 0.000 | 0.000 | 0.000 | 0.000 |
| 15 | 284 | DFRA | 11.240 | 5.585 | 0.225 | 0.120 | 1.000 | 1.000 | 0.000 | 0.000 | 0.000 | 0.000 | 0.000 |
| 16 | 295 | SOXS | 10.120 | 5.418 | 0.340 | 0.200 | 1.000 | 1.000 | 0.000 | 0.000 | 0.000 | 0.000 | 0.000 |
| 17 | 321 | TSNR | 10.560 | 6.678 | 0.154 | 0.101 | 1.000 | 1.000 | 0.000 | 0.000 | 0.000 | 0.000 | 0.000 |
| 18 | 413 | OMPD | 10.640 | 4.202 | 0.114 | 0.047 | 1.000 | 1.000 | 0.000 | 0.000 | 0.000 | 0.000 | 0.000 |
| 19 | 486 | LMRA | 9.000 | 3.651 | 0.176 | 0.077 | 1.000 | 1.000 | 0.000 | 0.000 | 0.000 | 0.000 | 0.000 |
| 20 | 505 | CARB | 10.040 | 5.827 | 0.124 | 0.078 | 1.000 | 1.000 | 0.000 | 0.000 | 0.000 | 0.000 | 0.000 |
| 21 | 506 | ANT3- | 10.880 | 5.126 | 0.151 | 0.078 | 1.000 | 0.920 | 0.080 | 0.000 | 0.000 | 0.000 | 0.000 |
| | | DPRIME | | | | | | | | | | | |
| 22 | 555 | QNRS | 13.200 | 6.770 | 0.228 | 0.117 | 1.000 | 0.320 | 0.680 | 0.000 | 0.000 | 0.000 | 0.000 |
| 23 | 558 | VANWI | 11.680 | 4.190 | 0.120 | 0.045 | 1.000 | 1.000 | 0.000 | 0.000 | 0.000 | 0.000 | 0.000 |
| 24 | 578 | TETX | 16.240 | 7.535 | 0.159 | 0.077 | 1.000 | 0.280 | 0.720 | 0.000 | 0.000 | 0.000 | 0.000 |
| 25 | 652 | VGA | 15.320 | 6.067 | 0.113 | 0.045 | 1.000 | 0.880 | 0.120 | 0.000 | 0.000 | 0.000 | 0.000 |
| 26 | 682 | MOX | 18.720 | 9.280 | 0.190 | 0.097 | 1.000 | 0.200 | 0.800 | 0.000 | 0.000 | 0.000 | 0.000 |
| 27 | 694 | ACT | 22.520 | 10.798 | 0.212 | 0.104 | 1.000 | 0.080 | 0.920 | 0.000 | 0.000 | 0.000 | 0.000 |
| 28 | 717 | VANRE | 10.600 | 4.311 | 0.178 | 0.074 | 1.000 | 1.000 | 0.000 | 0.000 | 0.000 | 0.000 | 0.000 |
| 29 | 749 | RMTB | 3.520 | 3.607 | 0.140 | 0.168 | 1.000 | 1.000 | 0.000 | 0.000 | 0.000 | 0.000 | 0.000 |
| 30 | 778 | DHA | 9.320 | 6.939 | 0.091 | 0.072 | 1.000 | 0.080 | 0.920 | 0.000 | 0.000 | 0.000 | 0.000 |
| 31 | 789 | CMY | 9.360 | 8.093 | 0.095 | 0.084 | 1.000 | 0.000 | 1.000 | 0.000 | 0.000 | 0.000 | 0.000 |
| 32 | 797 | FACT | 20.640 | 9.780 | 0.141 | 0.070 | 1.000 | 1.000 | 0.000 | 0.000 | 0.000 | 0.000 | 0.000 |
| 33 | 819 | OKP | 6.640 | 3.872 | 0.088 | 0.050 | 1.000 | 0.080 | 0.920 | 0.000 | 0.000 | 0.000 | 0.000 |
| 34 | 973 | IMP | 8.360 | 3.774 | 0.126 | 0.055 | 1.000 | 0.240 | 0.760 | 0.000 | 0.000 | 0.000 | 0.000 |
| 35 | 1048 | VIM | 4.160 | 1.491 | 0.058 | 0.020 | 1.000 | 0.080 | 0.920 | 0.000 | 0.000 | 0.000 | 0.000 |
| 36 | 1135 | PBP1B | 6.160 | 1.795 | 0.029 | 0.008 | 1.000 | 1.000 | 0.000 | 0.000 | 0.000 | 0.000 | 0.000 |
| 37 | 1146 | MPHE | 16.600 | 3.559 | 0.216 | 0.050 | 1.000 | 0.240 | 0.760 | 0.000 | 0.000 | 0.000 | 0.000 |
| 38 | 1182 | VATE | 9.200 | 6.007 | 0.157 | 0.104 | 1.000 | 0.680 | 0.320 | 0.000 | 0.000 | 0.000 | 0.000 |
| 39 | 1214 | OPRJ | 21.040 | 9.948 | 0.169 | 0.083 | 1.000 | 1.000 | 0.000 | 0.000 | 0.000 | 0.000 | 0.000 |
| 40 | 1254 | FOSB | 13.560 | 4.292 | 0.366 | 0.122 | 1.000 | 1.000 | 0.000 | 0.000 | 0.000 | 0.000 | 0.000 |
| 41 | 1271 | VPH | 10.760 | 4.447 | 0.138 | 0.060 | 1.000 | 1.000 | 0.000 | 0.000 | 0.000 | 0.000 | 0.000 |
| 42 | 1283 | SULI | 19.520 | 7.779 | 0.268 | 0.111 | 1.000 | 0.280 | 0.720 | 0.000 | 0.000 | 0.000 | 0.000 |
| 43 | 1297 | TET40 | 4.200 | 1.581 | 0.039 | 0.014 | 1.000 | 0.840 | 0.160 | 0.000 | 0.000 | 0.000 | 0.000 |
| 44 | 1389 | CPXAR | 10.840 | 4.469 | 0.173 | 0.077 | 1.000 | 0.320 | 0.680 | 0.000 | 0.000 | 0.000 | 0.000 |
| 45 | 1392 | AAC6- | 3.160 | 1.993 | 0.062 | 0.038 | 1.000 | 0.720 | 0.280 | 0.000 | 0.000 | 0.000 | 0.000 |
| | | PRIME | | | | | | | | | | | |
| 46 | 1422 | VGBB | 15.640 | 6.291 | 0.199 | 0.085 | 1.000 | 1.000 | 0.000 | 0.000 | 0.000 | 0.000 | 0.000 |
| 47 | 1440 | FOSC | 8.200 | 5.649 | 0.166 | 0.119 | 1.000 | 1.000 | 0.000 | 0.000 | 0.000 | 0.000 | 0.000 |
| 48 | 1535 | LNUA | 4.040 | 2.091 | 0.094 | 0.048 | 1.000 | 0.440 | 0.560 | 0.000 | 0.000 | 0.000 | 0.000 |
| 49 | 1569 | PARE | 15.240 | 8.997 | 0.095 | 0.057 | 1.000 | 0.800 | 0.200 | 0.000 | 0.000 | 0.000 | 0.000 |
| 50 | 1695 | NDM | 9.680 | 5.313 | 0.133 | 0.072 | 1.000 | 1.000 | 0.000 | 0.000 | 0.000 | 0.000 | 0.000 |
| 51 | 1702 | SPG | 9.040 | 9.889 | 0.122 | 0.137 | 1.000 | 1.000 | 0.000 | 0.000 | 0.000 | 0.000 | 0.000 |
| 52 | 1753 | VEB | 12.520 | 5.221 | 0.161 | 0.066 | 1.000 | 0.080 | 0.920 | 0.000 | 0.000 | 0.000 | 0.000 |
| 53 | 1953 | LNUB | 15.280 | 5.136 | 0.218 | 0.077 | 1.000 | 0.400 | 0.600 | 0.000 | 0.000 | 0.000 | 0.000 |
| 54 | 2026 | ERMA | 8.240 | 4.503 | 0.127 | 0.071 | 1.000 | 0.920 | 0.080 | 0.000 | 0.000 | 0.000 | 0.000 |
| 55 | 2357 | SULII | 9.920 | 7.371 | 0.135 | 0.105 | 1.000 | 0.040 | 0.960 | 0.000 | 0.000 | 0.000 | 0.000 |
| 56 | 2517 | TET37 | 7.600 | 3.488 | 0.255 | 0.127 | 1.000 | 1.000 | 0.000 | 0.000 | 0.000 | 0.000 | 0.000 |
| 57 | 2822 | EMRK | 17.600 | 5.583 | 0.189 | 0.063 | 1.000 | 1.000 | 0.000 | 0.000 | 0.000 | 0.000 | 0.000 |
| 58 | 2999 | MPHB | 11.920 | 6.006 | 0.150 | 0.077 | 1.000 | 1.000 | 0.000 | 0.000 | 0.000 | 0.000 | 0.000 |
| 59 | 3024 | VANYM | 12.840 | 3.508 | 0.212 | 0.060 | 1.000 | 1.000 | 0.000 | 0.000 | 0.000 | 0.000 | 0.000 |
| 60 | 3041 | MECC | 124.360 | 52.273 | 0.739 | 0.312 | 1.000 | 0.520 | 0.480 | 0.000 | 0.000 | 0.000 | 0.000 |
| 61 | 3128 | TUFAB | 12.080 | 8.103 | 0.118 | 0.083 | 1.000 | 0.640 | 0.360 | 0.000 | 0.000 | 0.000 | 0.000 |
| 62 | 3176 | AMRB | 25.400 | 14.751 | 0.096 | 0.056 | 1.000 | 1.000 | 0.000 | 0.000 | 0.000 | 0.000 | 0.000 |
| 63 | 3270 | IRI | 11.320 | 5.865 | 0.088 | 0.048 | 1.000 | 1.000 | 0.000 | 0.000 | 0.000 | 0.000 | 0.000 |
| 64 | 3314 | RPOB | 20.960 | 21.784 | 0.069 | 0.074 | 1.000 | 0.320 | 0.680 | 0.000 | 0.000 | 0.000 | 0.000 |
| 65 | 3332 | TET35 | 8.080 | 4.907 | 0.083 | 0.053 | 1.000 | 1.000 | 0.000 | 0.000 | 0.000 | 0.000 | 0.000 |
| 66 | 3370 | CFRA | 19.320 | 5.692 | 0.215 | 0.065 | 1.000 | 1.000 | 0.000 | 0.000 | 0.000 | 0.000 | 0.000 |
| 67 | 3513 | BRP | 9.040 | 2.865 | 0.251 | 0.086 | 1.000 | 1.000 | 0.000 | 0.000 | 0.000 | 0.000 | 0.000 |
| 68 | 3613 | APH3- | 15.760 | 5.101 | 0.227 | 0.074 | 1.000 | 1.000 | 0.000 | 0.000 | 0.000 | 0.000 | 0.000 |
| | | PRIME | | | | | | | | | | | |
| 69 | 3697 | TETM | 16.320 | 10.590 | 0.098 | 0.065 | 1.000 | 0.520 | 0.480 | 0.000 | 0.000 | 0.000 | 0.000 |
| 70 | 3778 | IND | 11.080 | 3.639 | 0.170 | 0.057 | 1.000 | 0.040 | 0.960 | 0.000 | 0.000 | 0.000 | 0.000 |

Simulation settings:
k-mers: ‘constant’, k = 12
Gene coverage: 1
Number of genes: 1
Errors: ‘off’
Penalty score: 0.1
Thresholding: multiplier = 1, all factors (1-on)
Entropy screening: ‘rand’

TABLE S6
Resistance genes simulations with variable-mer blocks centered around k = 10

| | | | Avg | StDev | Avg | StDev | | Identification level | | | | | |
|---|---|---|---|---|---|---|---|---|---|---|---|---|---|
| | Data- | Gene | block for | block for | coverage for | coverage for | | (fraction of trials) | | | | Avg | StDev |
| | base | sub- | identifi- | identifi- | identifi- | identifi- | Accu- | Specific | Sub- | | Incor- | false | false |
| No. | No. | class | cation | cation | cation | cation | racy | gene | class | Class | rect | positives | positives |
| 1 | 4 | VANZA | 19.120 | 8.268 | 0.350 | 0.158 | 1.000 | 1.000 | 0.000 | 0.000 | 0.000 | 0.000 | 0.000 |
| 2 | 52 | VANWG | 21.040 | 12.408 | 0.224 | 0.131 | 1.000 | 0.120 | 0.880 | 0.000 | 0.000 | 0.000 | 0.000 |
| 3 | 62 | CTX | 20.800 | 10.178 | 0.206 | 0.098 | 1.000 | 0.000 | 1.000 | 0.000 | 0.000 | 0.000 | 0.000 |
| 4 | 68 | MEFA | 31.960 | 15.624 | 0.231 | 0.114 | 1.000 | 0.560 | 0.440 | 0.000 | 0.000 | 0.000 | 0.000 |
| 5 | 76 | OXA | 21.440 | 10.587 | 0.228 | 0.111 | 1.000 | 0.000 | 1.000 | 0.000 | 0.000 | 0.000 | 0.000 |
| 6 | 92 | CATB | 15.320 | 6.460 | 0.207 | 0.085 | 1.000 | 0.120 | 0.880 | 0.000 | 0.000 | 0.000 | 0.000 |
| 7 | 162 | EREA | 22.240 | 11.530 | 0.157 | 0.080 | 1.000 | 0.360 | 0.640 | 0.000 | 0.000 | 0.000 | 0.000 |
| 8 | 174 | TEM | 21.280 | 7.591 | 0.221 | 0.079 | 1.000 | 0.000 | 1.000 | 0.000 | 0.000 | 0.000 | 0.000 |
| 9 | 193 | CML | 18.840 | 9.150 | 0.124 | 0.060 | 1.000 | 0.200 | 0.800 | 0.000 | 0.000 | 0.000 | 0.000 |
| 10 | 207 | NIMA | 10.560 | 4.360 | 0.195 | 0.082 | 1.000 | 0.960 | 0.040 | 0.000 | 0.000 | 0.000 | 0.000 |
| 11 | 239 | DHFR | 12.400 | 4.882 | 0.227 | 0.090 | 1.000 | 0.320 | 0.680 | 0.000 | 0.000 | 0.000 | 0.000 |
| 12 | 246 | FOLP | 31.960 | 21.458 | 0.246 | 0.165 | 1.000 | 0.920 | 0.080 | 0.000 | 0.000 | 0.000 | 0.000 |
| 13 | 274 | PARC | 49.040 | 20.733 | 0.191 | 0.080 | 1.000 | 0.200 | 0.800 | 0.000 | 0.000 | 0.000 | 0.000 |
| 14 | 276 | SHV | 22.560 | 9.933 | 0.228 | 0.105 | 1.000 | 0.080 | 0.920 | 0.000 | 0.000 | 0.000 | 0.000 |
| 15 | 284 | DFRA | 19.280 | 8.872 | 0.304 | 0.153 | 1.000 | 1.000 | 0.000 | 0.000 | 0.000 | 0.000 | 0.000 |
| 16 | 295 | SOXS | 13.200 | 4.770 | 0.353 | 0.134 | 1.000 | 1.000 | 0.000 | 0.000 | 0.000 | 0.000 | 0.000 |
| 17 | 321 | TSNR | 21.120 | 10.113 | 0.237 | 0.114 | 1.000 | 1.000 | 0.000 | 0.000 | 0.000 | 0.000 | 0.000 |
| 18 | 413 | OMPD | 23.360 | 9.827 | 0.189 | 0.078 | 1.000 | 1.000 | 0.000 | 0.000 | 0.000 | 0.000 | 0.000 |
| 19 | 486 | LMRA | 11.760 | 5.904 | 0.177 | 0.085 | 1.000 | 1.000 | 0.000 | 0.000 | 0.000 | 0.000 | 0.000 |
| 20 | 505 | CARB | 20.040 | 10.470 | 0.193 | 0.098 | 1.000 | 1.000 | 0.000 | 0.000 | 0.000 | 0.000 | 0.000 |
| 21 | 506 | ANT3- | 20.000 | 7.303 | 0.216 | 0.079 | 1.000 | 0.920 | 0.080 | 0.000 | 0.000 | 0.000 | 0.000 |
| | | DPRIME | | | | | | | | | | | |
| 22 | 555 | QNRS | 17.280 | 7.056 | 0.223 | 0.088 | 1.000 | 0.480 | 0.520 | 0.000 | 0.000 | 0.000 | 0.000 |
| 23 | 558 | VANWI | 22.360 | 10.012 | 0.170 | 0.078 | 1.000 | 1.000 | 0.000 | 0.000 | 0.000 | 0.000 | 0.000 |
| 24 | 578 | TETX | 39.200 | 17.830 | 0.296 | 0.136 | 1.000 | 0.400 | 0.600 | 0.000 | 0.000 | 0.000 | 0.000 |
| 25 | 652 | VGA | 34.680 | 12.730 | 0.198 | 0.074 | 1.000 | 0.800 | 0.200 | 0.000 | 0.000 | 0.000 | 0.000 |
| 26 | 682 | MOX | 33.680 | 15.063 | 0.254 | 0.111 | 1.000 | 0.240 | 0.760 | 0.000 | 0.000 | 0.000 | 0.000 |
| 27 | 694 | ACT | 35.720 | 17.021 | 0.247 | 0.125 | 1.000 | 0.080 | 0.920 | 0.000 | 0.000 | 0.000 | 0.000 |
| 28 | 717 | VANRE | 25.720 | 11.516 | 0.334 | 0.151 | 1.000 | 1.000 | 0.000 | 0.000 | 0.000 | 0.000 | 0.000 |
| 29 | 749 | RMTB | 7.880 | 4.324 | 0.274 | 0.153 | 1.000 | 0.920 | 0.080 | 0.000 | 0.000 | 0.000 | 0.000 |
| 30 | 778 | DHA | 21.640 | 10.950 | 0.165 | 0.081 | 1.000 | 0.040 | 0.960 | 0.000 | 0.000 | 0.000 | 0.000 |
| 31 | 789 | CMY | 23.080 | 14.130 | 0.174 | 0.112 | 1.000 | 0.000 | 1.000 | 0.000 | 0.000 | 0.000 | 0.000 |
| 32 | 797 | FACT | 36.240 | 27.470 | 0.193 | 0.145 | 1.000 | 1.000 | 0.000 | 0.000 | 0.000 | 0.000 | 0.000 |
| 33 | 819 | OKP | 27.560 | 13.482 | 0.283 | 0.142 | 1.000 | 0.080 | 0.920 | 0.000 | 0.000 | 0.000 | 0.000 |
| 34 | 973 | IMP | 16.640 | 7.262 | 0.199 | 0.082 | 1.000 | 0.160 | 0.840 | 0.000 | 0.000 | 0.000 | 0.000 |
| 35 | 1048 | VIM | 15.280 | 6.175 | 0.164 | 0.068 | 1.000 | 0.040 | 0.960 | 0.000 | 0.000 | 0.000 | 0.000 |
| 36 | 1135 | PBP1B | 31.200 | 14.491 | 0.110 | 0.050 | 1.000 | 1.000 | 0.000 | 0.000 | 0.000 | 0.000 | 0.000 |
| 37 | 1146 | MPHE | 25.120 | 14.432 | 0.251 | 0.145 | 1.000 | 0.360 | 0.640 | 0.000 | 0.000 | 0.000 | 0.000 |
| 38 | 1182 | VATE | 13.800 | 7.439 | 0.188 | 0.101 | 1.000 | 0.480 | 0.520 | 0.000 | 0.000 | 0.000 | 0.000 |
| 39 | 1214 | OPRJ | 50.280 | 25.842 | 0.310 | 0.158 | 1.000 | 1.000 | 0.000 | 0.000 | 0.000 | 0.000 | 0.000 |
| 40 | 1254 | FOSB | 18.320 | 8.265 | 0.387 | 0.173 | 1.000 | 0.960 | 0.040 | 0.000 | 0.000 | 0.000 | 0.000 |
| 41 | 1271 | VPH | 25.520 | 13.080 | 0.264 | 0.137 | 1.000 | 1.000 | 0.000 | 0.000 | 0.000 | 0.000 | 0.000 |
| 42 | 1283 | SULI | 40.680 | 19.786 | 0.439 | 0.240 | 1.000 | 0.120 | 0.880 | 0.000 | 0.000 | 0.000 | 0.000 |
| 43 | 1297 | TET40 | 17.560 | 8.466 | 0.125 | 0.060 | 1.000 | 0.360 | 0.640 | 0.000 | 0.000 | 0.000 | 0.000 |
| 44 | 1389 | CPXAR | 19.240 | 8.141 | 0.234 | 0.096 | 1.000 | 0.400 | 0.600 | 0.000 | 0.000 | 0.000 | 0.000 |
| 45 | 1392 | AAC6- | 10.680 | 4.589 | 0.167 | 0.072 | 1.000 | 0.680 | 0.320 | 0.000 | 0.000 | 0.000 | 0.000 |
| | | PRIME | | | | | | | | | | | |
| 46 | 1422 | VGBB | 26.480 | 10.813 | 0.260 | 0.105 | 1.000 | 1.000 | 0.000 | 0.000 | 0.000 | 0.000 | 0.000 |
| 47 | 1440 | FOSC | 14.520 | 7.343 | 0.223 | 0.112 | 1.000 | 1.000 | 0.000 | 0.000 | 0.000 | 0.000 | 0.000 |
| 48 | 1535 | LNUA | 10.400 | 4.444 | 0.196 | 0.080 | 1.000 | 0.440 | 0.560 | 0.000 | 0.000 | 0.000 | 0.000 |
| 49 | 1569 | PARE | 38.560 | 13.863 | 0.179 | 0.065 | 1.000 | 0.640 | 0.360 | 0.000 | 0.000 | 0.000 | 0.000 |
| 50 | 1695 | NDM | 22.400 | 7.303 | 0.238 | 0.077 | 1.000 | 1.000 | 0.000 | 0.000 | 0.000 | 0.000 | 0.000 |
| 51 | 1702 | SPG | 19.400 | 12.295 | 0.197 | 0.128 | 1.000 | 1.000 | 0.000 | 0.000 | 0.000 | 0.000 | 0.000 |
| 52 | 1753 | VEB | 28.760 | 17.408 | 0.286 | 0.175 | 1.000 | 0.040 | 0.960 | 0.000 | 0.000 | 0.000 | 0.000 |
| 53 | 1953 | LNUB | 29.760 | 15.517 | 0.334 | 0.177 | 1.000 | 0.640 | 0.360 | 0.000 | 0.000 | 0.000 | 0.000 |
| 54 | 2026 | ERMA | 22.400 | 10.704 | 0.272 | 0.128 | 1.000 | 0.720 | 0.280 | 0.000 | 0.000 | 0.000 | 0.000 |
| 55 | 2357 | SULII | 20.800 | 10.165 | 0.222 | 0.108 | 1.000 | 0.160 | 0.840 | 0.000 | 0.000 | 0.000 | 0.000 |
| 56 | 2517 | TET37 | 9.560 | 4.620 | 0.248 | 0.116 | 1.000 | 1.000 | 0.000 | 0.000 | 0.000 | 0.000 | 0.000 |
| 57 | 2822 | EMRK | 30.040 | 12.684 | 0.250 | 0.108 | 1.000 | 1.000 | 0.000 | 0.000 | 0.000 | 0.000 | 0.000 |
| 58 | 2999 | MPHB | 22.880 | 8.710 | 0.219 | 0.082 | 1.000 | 0.960 | 0.040 | 0.000 | 0.000 | 0.000 | 0.000 |
| 59 | 3024 | VANYM | 21.800 | 9.954 | 0.273 | 0.132 | 1.000 | 1.000 | 0.000 | 0.000 | 0.000 | 0.000 | 0.000 |
| 60 | 3041 | MECC | 127.480 | 77.287 | 0.624 | 0.394 | 1.000 | 0.560 | 0.440 | 0.000 | 0.000 | 0.000 | 0.000 |
| 61 | 3128 | TUFAB | 25.120 | 11.344 | 0.182 | 0.081 | 1.000 | 0.560 | 0.440 | 0.000 | 0.000 | 0.000 | 0.000 |
| 62 | 3176 | AMRB | 60.080 | 30.228 | 0.169 | 0.084 | 1.000 | 1.000 | 0.000 | 0.000 | 0.000 | 0.000 | 0.000 |
| 63 | 3270 | IRI | 44.600 | 17.550 | 0.267 | 0.107 | 1.000 | 1.000 | 0.000 | 0.000 | 0.000 | 0.000 | 0.000 |
| 64 | 3314 | RPOB | 46.760 | 17.439 | 0.114 | 0.043 | 1.000 | 0.080 | 0.920 | 0.000 | 0.000 | 0.080 | 0.277 |
| 65 | 3332 | TET35 | 19.320 | 8.697 | 0.148 | 0.066 | 1.000 | 1.000 | 0.000 | 0.000 | 0.000 | 0.000 | 0.000 |
| 66 | 3370 | CFRA | 35.440 | 13.476 | 0.301 | 0.116 | 1.000 | 1.000 | 0.000 | 0.000 | 0.000 | 0.000 | 0.000 |
| 67 | 3513 | BRP | 9.760 | 4.893 | 0.212 | 0.107 | 1.000 | 1.000 | 0.000 | 0.000 | 0.000 | 0.000 | 0.000 |
| 68 | 3613 | APH3- | 24.520 | 9.005 | 0.269 | 0.098 | 1.000 | 0.960 | 0.040 | 0.000 | 0.000 | 0.000 | 0.000 |
| | | PRIME | | | | | | | | | | | |
| 69 | 3697 | TETM | 47.120 | 20.376 | 0.219 | 0.094 | 1.000 | 0.600 | 0.400 | 0.000 | 0.000 | 0.000 | 0.000 |
| 70 | 3778 | IND | 17.160 | 6.743 | 0.208 | 0.079 | 1.000 | 0.040 | 0.960 | 0.000 | 0.000 | 0.000 | 0.000 |

Simulation settings:
k-mers: ‘variable’, k = 10
Gene coverage: 1
Number of genes: 1
Errors: ‘off’
Penalty score: 0.1
Thresholding: multiplier = 1, all factors (1-on)
Entropy screening: ‘rand’

TABLE S7
Resistance genes simulations with 0.01 (2%) error rate

| | | | Avg | StDev | Avg | StDev | | Identification level | | | | | |
|---|---|---|---|---|---|---|---|---|---|---|---|---|---|
| | Data- | Gene | block for | block for | coverage for | coverage for | | (fraction of trials) | | | | Avg | StDev |
| | base | sub- | identifi- | identifi- | identifi- | identifi- | Accu- | Specific | Sub- | | Incor- | false | false |
| No. | No. | class | cation | cation | cation | cation | racy | gene | class | Class | rect | positives | positives |
| 1 | 4 | VANZA | 23.500 | 12.786 | 0.478 | 0.262 | 0.960 | 0.960 | 0.000 | 0.000 | 0.040 | 0.000 | 0.000 |
| 2 | 52 | VANWG | 47.280 | 18.573 | 0.555 | 0.218 | 1.000 | 0.400 | 0.600 | 0.000 | 0.000 | 0.000 | 0.000 |
| 3 | 62 | CTX | 56.480 | 12.600 | 0.640 | 0.144 | 1.000 | 0.000 | 1.000 | 0.000 | 0.000 | 0.040 | 0.200 |
| 4 | 68 | MEFA | 78.560 | 17.280 | 0.640 | 0.141 | 1.000 | 0.640 | 0.360 | 0.000 | 0.000 | 0.000 | 0.000 |
| 5 | 76 | OXA | 46.320 | 16.175 | 0.556 | 0.194 | 1.000 | 0.040 | 0.960 | 0.000 | 0.000 | 0.000 | 0.000 |
| 6 | 92 | CATB | 18.880 | 12.601 | 0.291 | 0.195 | 1.000 | 0.280 | 0.720 | 0.000 | 0.000 | 0.000 | 0.000 |
| 7 | 162 | EREA | 26.440 | 15.578 | 0.214 | 0.126 | 1.000 | 0.480 | 0.520 | 0.000 | 0.000 | 0.000 | 0.000 |
| 8 | 174 | TEM | 35.960 | 7.829 | 0.424 | 0.093 | 1.000 | 0.280 | 0.720 | 0.000 | 0.000 | 0.000 | 0.000 |
| 9 | 193 | CML | 66.320 | 17.587 | 0.500 | 0.133 | 1.000 | 0.240 | 0.720 | 0.040 | 0.000 | 0.000 | 0.000 |
| 10 | 207 | NIMA | 12.720 | 6.222 | 0.272 | 0.136 | 1.000 | 1.000 | 0.000 | 0.000 | 0.000 | 0.000 | 0.000 |
| 11 | 239 | DHFR | 26.000 | 7.348 | 0.536 | 0.154 | 1.000 | 0.080 | 0.920 | 0.000 | 0.000 | 0.000 | 0.000 |
| 12 | 246 | FOLP | 90.174 | 16.420 | 0.755 | 0.140 | 0.920 | 0.920 | 0.000 | 0.000 | 0.080 | 0.000 | 0.000 |
| 13 | 274 | PARC | 189.333 | 43.389 | 0.849 | 0.196 | 0.840 | 0.040 | 0.520 | 0.280 | 0.160 | 1.560 | 2.142 |
| 14 | 276 | SHV | 78.840 | 12.229 | 0.899 | 0.140 | 1.000 | 0.040 | 0.960 | 0.000 | 0.000 | 0.560 | 0.583 |
| 15 | 284 | DFRA | 30.880 | 9.198 | 0.546 | 0.165 | 1.000 | 1.000 | 0.000 | 0.000 | 0.000 | 0.000 | 0.000 |
| 16 | 295 | SOXS | 17.360 | 6.794 | 0.520 | 0.208 | 1.000 | 1.000 | 0.000 | 0.000 | 0.000 | 0.000 | 0.000 |
| 17 | 321 | TSNR | 47.833 | 12.426 | 0.603 | 0.157 | 0.960 | 0.960 | 0.000 | 0.000 | 0.040 | 0.000 | 0.000 |
| 18 | 413 | OMPD | 57.250 | 9.755 | 0.528 | 0.090 | 0.960 | 0.960 | 0.000 | 0.000 | 0.040 | 0.000 | 0.000 |
| 19 | 486 | LMRA | 20.280 | 8.463 | 0.350 | 0.144 | 1.000 | 1.000 | 0.000 | 0.000 | 0.000 | 0.000 | 0.000 |
| 20 | 505 | CARB | 52.200 | 8.446 | 0.574 | 0.092 | 1.000 | 0.960 | 0.000 | 0.040 | 0.000 | 0.000 | 0.000 |
| 21 | 506 | ANT3- | 41.880 | 13.318 | 0.519 | 0.168 | 1.000 | 0.920 | 0.080 | 0.000 | 0.000 | 0.240 | 0.831 |
| | | DPRIME | | | | | | | | | | | |
| 22 | 555 | QNRS | 35.840 | 5.749 | 0.534 | 0.093 | 1.000 | 0.600 | 0.400 | 0.000 | 0.000 | 0.000 | 0.000 |
| 23 | 558 | VANWI | 31.520 | 10.190 | 0.277 | 0.090 | 1.000 | 1.000 | 0.000 | 0.000 | 0.000 | 0.000 | 0.000 |
| 24 | 578 | TETX | 84.280 | 14.932 | 0.717 | 0.129 | 1.000 | 0.840 | 0.160 | 0.000 | 0.000 | 0.080 | 0.277 |
| 25 | 652 | VGA | 101.040 | 36.600 | 0.639 | 0.231 | 1.000 | 0.920 | 0.080 | 0.000 | 0.000 | 0.560 | 1.960 |
| 26 | 682 | MOX | 96.800 | 16.889 | 0.836 | 0.145 | 1.000 | 0.440 | 0.560 | 0.000 | 0.000 | 0.280 | 0.458 |
| 27 | 694 | ACT | 86.240 | 14.878 | 0.686 | 0.119 | 1.000 | 0.240 | 0.760 | 0.000 | 0.000 | 0.040 | 0.200 |
| 28 | 717 | VANRE | 51.083 | 9.329 | 0.727 | 0.133 | 0.960 | 0.960 | 0.000 | 0.000 | 0.040 | 0.000 | 0.000 |
| 29 | 749 | RMTB | 10.542 | 3.538 | 0.394 | 0.138 | 0.960 | 0.960 | 0.000 | 0.000 | 0.040 | 0.000 | 0.000 |
| 30 | 778 | DHA | 48.080 | 16.457 | 0.416 | 0.144 | 1.000 | 0.040 | 0.960 | 0.000 | 0.000 | 0.000 | 0.000 |
| 31 | 789 | CMY | 33.520 | 14.477 | 0.290 | 0.126 | 1.000 | 0.000 | 1.000 | 0.000 | 0.000 | 0.000 | 0.000 |
| 32 | 797 | FACT | 139.222 | 17.795 | 0.823 | 0.106 | 0.720 | 0.680 | 0.040 | 0.000 | 0.280 | 0.080 | 0.400 |
| 33 | 819 | OKP | 65.583 | 9.169 | 0.751 | 0.106 | 0.960 | 0.120 | 0.840 | 0.000 | 0.040 | 0.080 | 0.277 |
| 34 | 973 | IMP | 28.400 | 16.946 | 0.378 | 0.225 | 1.000 | 0.240 | 0.760 | 0.000 | 0.000 | 0.000 | 0.000 |
| 35 | 1048 | VIM | 31.920 | 12.945 | 0.390 | 0.158 | 1.000 | 0.040 | 0.960 | 0.000 | 0.000 | 0.000 | 0.000 |
| 36 | 1135 | PBP1B | 79.000 | 58.407 | 0.319 | 0.235 | 0.840 | 0.760 | 0.040 | 0.040 | 0.160 | 0.040 | 0.200 |
| 37 | 1146 | MPHE | 69.000 | 6.285 | 0.774 | 0.071 | 1.000 | 0.440 | 0.560 | 0.000 | 0.000 | 0.000 | 0.000 |
| 38 | 1182 | VATE | 39.917 | 8.387 | 0.611 | 0.129 | 0.960 | 0.640 | 0.320 | 0.000 | 0.040 | 0.000 | 0.000 |
| 39 | 1214 | OPRJ | 130.773 | 14.784 | 0.903 | 0.103 | 0.880 | 0.480 | 0.400 | 0.000 | 0.120 | 0.480 | 0.586 |
| 40 | 1254 | FOSB | 31.120 | 9.816 | 0.722 | 0.229 | 1.000 | 0.800 | 0.200 | 0.000 | 0.000 | 0.000 | 0.000 |
| 41 | 1271 | VPH | 46.208 | 28.717 | 0.530 | 0.330 | 0.960 | 0.920 | 0.040 | 0.000 | 0.040 | 0.080 | 0.277 |
| 42 | 1283 | SULI | 61.080 | 9.617 | 0.717 | 0.116 | 1.000 | 0.240 | 0.760 | 0.000 | 0.000 | 0.040 | 0.200 |
| 43 | 1297 | TET40 | 51.960 | 30.194 | 0.420 | 0.245 | 1.000 | 0.640 | 0.360 | 0.000 | 0.000 | 0.000 | 0.000 |
| 44 | 1389 | CPXAR | 31.320 | 11.089 | 0.438 | 0.159 | 1.000 | 0.320 | 0.640 | 0.040 | 0.000 | 0.360 | 1.440 |
| 45 | 1392 | AAC6- | 11.240 | 7.639 | 0.198 | 0.136 | 1.000 | 0.760 | 0.240 | 0.000 | 0.000 | 0.000 | 0.000 |
| | | PRIME | | | | | | | | | | | |
| 46 | 1422 | VGBB | 64.750 | 8.828 | 0.723 | 0.100 | 0.960 | 0.920 | 0.040 | 0.000 | 0.040 | 0.120 | 0.600 |
| 47 | 1440 | FOSC | 24.920 | 7.433 | 0.438 | 0.131 | 1.000 | 1.000 | 0.000 | 0.000 | 0.000 | 0.000 | 0.000 |
| 48 | 1535 | LNUA | 13.960 | 9.312 | 0.285 | 0.190 | 1.000 | 0.720 | 0.280 | 0.000 | 0.000 | 0.000 | 0.000 |
| 49 | 1569 | PARE | 136.143 | 36.753 | 0.719 | 0.195 | 0.280 | 0.200 | 0.080 | 0.000 | 0.720 | 0.680 | 2.358 |
| 50 | 1695 | NDM | 45.875 | 9.396 | 0.557 | 0.114 | 0.960 | 0.960 | 0.000 | 0.000 | 0.040 | 0.000 | 0.000 |
| 51 | 1702 | SPG | 60.667 | 10.655 | 0.700 | 0.124 | 0.960 | 0.920 | 0.040 | 0.000 | 0.040 | 0.040 | 0.200 |
| 52 | 1753 | VEB | 76.520 | 9.430 | 0.844 | 0.103 | 1.000 | 0.000 | 1.000 | 0.000 | 0.000 | 0.040 | 0.200 |
| 53 | 1953 | LNUB | 63.760 | 6.437 | 0.786 | 0.080 | 1.000 | 0.960 | 0.040 | 0.000 | 0.000 | 0.000 | 0.000 |
| 54 | 2026 | ERMA | 47.000 | 18.815 | 0.633 | 0.254 | 1.000 | 0.880 | 0.080 | 0.040 | 0.000 | 0.200 | 0.500 |
| 55 | 2357 | SULII | 53.800 | 10.079 | 0.654 | 0.124 | 1.000 | 0.080 | 0.920 | 0.000 | 0.000 | 0.080 | 0.277 |
| 56 | 2517 | TET37 | 18.958 | 2.911 | 0.560 | 0.091 | 0.960 | 0.960 | 0.000 | 0.000 | 0.040 | 0.000 | 0.000 |
| 57 | 2822 | EMRK | 71.920 | 12.566 | 0.676 | 0.120 | 1.000 | 0.920 | 0.000 | 0.080 | 0.000 | 0.320 | 1.145 |
| 58 | 2999 | MPHB | 57.120 | 8.550 | 0.619 | 0.096 | 1.000 | 1.000 | 0.000 | 0.000 | 0.000 | 0.000 | 0.000 |
| 59 | 3024 | VANYM | 51.640 | 7.979 | 0.724 | 0.114 | 1.000 | 0.960 | 0.040 | 0.000 | 0.000 | 0.040 | 0.200 |
| 60 | 3041 | MECC | 175.360 | 40.240 | 0.870 | 0.200 | 1.000 | 0.440 | 0.560 | 0.000 | 0.000 | 0.000 | 0.000 |
| 61 | 3128 | TUFAB | 51.833 | 21.184 | 0.434 | 0.178 | 0.960 | 0.760 | 0.200 | 0.000 | 0.040 | 0.000 | 0.000 |
| 62 | 3176 | AMRB | 296.600 | 54.895 | 0.943 | 0.175 | 1.000 | 0.040 | 0.840 | 0.120 | 0.000 | 0.080 | 0.400 |
| 63 | 3270 | IRI | 125.708 | 22.542 | 0.866 | 0.156 | 0.960 | 0.520 | 0.440 | 0.000 | 0.040 | 0.480 | 0.586 |
| 64 | 3314 | RPOB | 336.286 | 52.159 | 0.944 | 0.147 | 0.280 | 0.000 | 0.280 | 0.000 | 0.720 | 1.480 | 2.756 |
| 65 | 3332 | TET35 | 43.043 | 9.943 | 0.383 | 0.088 | 0.920 | 0.920 | 0.000 | 0.000 | 0.080 | 0.000 | 0.000 |
| 66 | 3370 | CFRA | 78.773 | 13.245 | 0.745 | 0.124 | 0.880 | 0.840 | 0.040 | 0.000 | 0.120 | 0.040 | 0.200 |
| 67 | 3513 | BRP | 15.440 | 8.150 | 0.373 | 0.198 | 1.000 | 1.000 | 0.000 | 0.000 | 0.000 | 0.000 | 0.000 |
| 68 | 3613 | APH3- | 56.800 | 4.213 | 0.698 | 0.053 | 1.000 | 1.000 | 0.000 | 0.000 | 0.000 | 0.000 | 0.000 |
| | | PRIME | | | | | | | | | | | |
| 69 | 3697 | TETM | 168.400 | 19.807 | 0.872 | 0.103 | 1.000 | 0.760 | 0.240 | 0.000 | 0.000 | 0.360 | 0.700 |
| 70 | 3778 | IND | 51.680 | 10.523 | 0.695 | 0.143 | 1.000 | 0.080 | 0.920 | 0.000 | 0.000 | 0.000 | 0.000 |

Simulation settings:
k-mers: ‘constant’, k = 10
Gene coverage: 1
Number of genes: 1
Errors: ‘on’, 0.01
Penalty score: 0.1
Thresholding: multiplier = 2, all factors (1-on)
Entropy screening: ‘rand’

TABLE S8
Resistance genes simulations with 0.025 (5%) error rate

| | | | Avg | StDev | Avg | StDev | | Identification level | | | | | |
|---|---|---|---|---|---|---|---|---|---|---|---|---|---|
| | Data- | Gene | block for | block for | coverage for | coverage for | | (fraction of trials) | | | | Avg | StDev |
| | base | sub- | identifi- | identifi- | identifi- | identifi- | Accu- | Specific | Sub- | | Incor- | false | false |
| No. | No. | class | cation | cation | cation | cation | racy | gene | class | Class | rect | positives | positives |
| 1 | 4 | VANZA | 25.250 | 10.707 | 0.514 | 0.218 | 0.960 | 0.960 | 0.000 | 0.000 | 0.040 | 0.040 | 0.200 |
| 2 | 52 | VANWG | 53.960 | 14.438 | 0.633 | 0.170 | 1.000 | 0.440 | 0.560 | 0.000 | 0.000 | 0.000 | 0.000 |
| 3 | 62 | CTX | 60.440 | 21.370 | 0.686 | 0.243 | 1.000 | 0.000 | 1.000 | 0.000 | 0.000 | 0.080 | 0.277 |
| 4 | 68 | MEFA | 85.040 | 11.040 | 0.694 | 0.090 | 1.000 | 0.560 | 0.440 | 0.000 | 0.000 | 0.040 | 0.200 |
| 5 | 76 | OXA | 44.800 | 19.462 | 0.538 | 0.235 | 1.000 | 0.000 | 1.000 | 0.000 | 0.000 | 0.200 | 1.000 |
| 6 | 92 | CATB | 24.160 | 13.966 | 0.373 | 0.218 | 1.000 | 0.360 | 0.640 | 0.000 | 0.000 | 0.000 | 0.000 |
| 7 | 162 | EREA | 25.174 | 15.614 | 0.204 | 0.125 | 0.920 | 0.520 | 0.400 | 0.000 | 0.080 | 0.040 | 0.200 |
| 8 | 174 | TEM | 44.320 | 16.663 | 0.525 | 0.198 | 1.000 | 0.120 | 0.880 | 0.000 | 0.000 | 0.160 | 0.374 |
| 9 | 193 | CML | 61.840 | 26.100 | 0.467 | 0.197 | 1.000 | 0.160 | 0.800 | 0.040 | 0.000 | 0.040 | 0.200 |
| 10 | 207 | NIMA | 17.375 | 10.034 | 0.373 | 0.219 | 0.960 | 0.920 | 0.040 | 0.000 | 0.040 | 0.000 | 0.000 |
| 11 | 239 | DHFR | 24.800 | 8.860 | 0.513 | 0.183 | 1.000 | 0.320 | 0.680 | 0.000 | 0.000 | 0.000 | 0.000 |
| 12 | 246 | FOLP | 89.080 | 24.605 | 0.746 | 0.208 | 1.000 | 0.920 | 0.080 | 0.000 | 0.000 | 0.080 | 0.277 |
| 13 | 274 | PARC | 202.778 | 34.739 | 0.909 | 0.157 | 0.720 | 0.080 | 0.400 | 0.240 | 0.280 | 2.080 | 2.465 |
| 14 | 276 | SHV | 76.640 | 17.464 | 0.874 | 0.198 | 1.000 | 0.000 | 1.000 | 0.000 | 0.000 | 1.000 | 1.958 |
| 15 | 284 | DFRA | 33.480 | 11.748 | 0.594 | 0.211 | 1.000 | 0.920 | 0.080 | 0.000 | 0.000 | 0.240 | 0.597 |
| 16 | 295 | SOXS | 18.560 | 5.752 | 0.556 | 0.177 | 1.000 | 0.960 | 0.040 | 0.000 | 0.000 | 0.240 | 1.200 |
| 17 | 321 | TSNR | 57.280 | 13.107 | 0.723 | 0.167 | 1.000 | 0.880 | 0.120 | 0.000 | 0.000 | 0.280 | 0.542 |
| 18 | 413 | OMPD | 63.292 | 12.896 | 0.584 | 0.120 | 0.960 | 0.920 | 0.040 | 0.000 | 0.040 | 0.040 | 0.200 |
| 19 | 486 | LMRA | 24.708 | 7.214 | 0.422 | 0.124 | 0.960 | 0.960 | 0.000 | 0.000 | 0.040 | 0.000 | 0.000 |
| 20 | 505 | CARB | 54.200 | 18.538 | 0.598 | 0.204 | 1.000 | 0.880 | 0.040 | 0.080 | 0.000 | 0.320 | 0.900 |
| 21 | 506 | ANT3- | 44.080 | 12.486 | 0.547 | 0.157 | 1.000 | 0.920 | 0.080 | 0.000 | 0.000 | 0.240 | 1.200 |
| | | DPRIME | | | | | | | | | | | |
| 22 | 555 | QNRS | 39.080 | 6.416 | 0.588 | 0.098 | 1.000 | 0.480 | 0.520 | 0.000 | 0.000 | 0.080 | 0.277 |
| 23 | 558 | VANWI | 40.760 | 10.345 | 0.358 | 0.092 | 1.000 | 1.000 | 0.000 | 0.000 | 0.000 | 0.040 | 0.200 |
| 24 | 578 | TETX | 89.960 | 18.620 | 0.767 | 0.161 | 1.000 | 0.680 | 0.320 | 0.000 | 0.000 | 0.200 | 0.408 |
| 25 | 652 | VGA | 102.120 | 32.917 | 0.646 | 0.208 | 1.000 | 0.920 | 0.080 | 0.000 | 0.000 | 0.120 | 0.332 |
| 26 | 682 | MOX | 106.080 | 13.617 | 0.918 | 0.119 | 1.000 | 0.240 | 0.760 | 0.000 | 0.000 | 0.640 | 0.638 |
| 27 | 694 | ACT | 94.800 | 17.424 | 0.756 | 0.140 | 1.000 | 0.360 | 0.640 | 0.000 | 0.000 | 0.160 | 0.374 |
| 28 | 717 | VANRE | 56.720 | 7.992 | 0.808 | 0.116 | 1.000 | 0.880 | 0.120 | 0.000 | 0.000 | 0.160 | 0.374 |
| 29 | 749 | RMTB | 11.440 | 6.905 | 0.433 | 0.265 | 1.000 | 0.880 | 0.120 | 0.000 | 0.000 | 0.520 | 2.220 |
| 30 | 778 | DHA | 41.760 | 17.429 | 0.361 | 0.151 | 1.000 | 0.000 | 1.000 | 0.000 | 0.000 | 0.080 | 0.400 |
| 31 | 789 | CMY | 38.640 | 21.022 | 0.335 | 0.183 | 1.000 | 0.000 | 0.960 | 0.040 | 0.000 | 0.000 | 0.000 |
| 32 | 797 | FACT | 147.944 | 22.161 | 0.875 | 0.132 | 0.720 | 0.480 | 0.240 | 0.000 | 0.280 | 0.280 | 0.542 |
| 33 | 819 | OKP | 79.375 | 9.050 | 0.911 | 0.105 | 0.960 | 0.120 | 0.800 | 0.040 | 0.040 | 0.520 | 0.653 |
| 34 | 973 | IMP | 42.040 | 17.862 | 0.557 | 0.239 | 1.000 | 0.120 | 0.880 | 0.000 | 0.000 | 0.040 | 0.200 |
| 35 | 1048 | VIM | 36.840 | 12.233 | 0.450 | 0.151 | 1.000 | 0.000 | 1.000 | 0.000 | 0.000 | 0.000 | 0.000 |
| 36 | 1135 | PBP1B | 87.389 | 31.797 | 0.353 | 0.128 | 0.720 | 0.720 | 0.000 | 0.000 | 0.280 | 0.080 | 0.400 |
| 37 | 1146 | MPHE | 69.833 | 7.063 | 0.783 | 0.080 | 0.960 | 0.560 | 0.360 | 0.040 | 0.040 | 0.280 | 1.400 |
| 38 | 1182 | VATE | 44.200 | 8.067 | 0.678 | 0.124 | 1.000 | 0.520 | 0.480 | 0.000 | 0.000 | 0.040 | 0.200 |
| 39 | 1214 | OPRJ | 138.304 | 15.423 | 0.955 | 0.107 | 0.920 | 0.200 | 0.720 | 0.000 | 0.080 | 0.880 | 0.726 |
| 40 | 1254 | FOSB | 34.480 | 7.495 | 0.805 | 0.175 | 1.000 | 0.960 | 0.040 | 0.000 | 0.000 | 0.000 | 0.000 |
| 41 | 1271 | VPH | 52.957 | 25.121 | 0.607 | 0.289 | 0.920 | 0.840 | 0.080 | 0.000 | 0.080 | 0.200 | 0.577 |
| 42 | 1283 | SULI | 65.840 | 12.202 | 0.774 | 0.145 | 1.000 | 0.120 | 0.880 | 0.000 | 0.000 | 0.240 | 0.663 |
| 43 | 1297 | TET40 | 35.833 | 24.925 | 0.290 | 0.201 | 0.960 | 0.400 | 0.560 | 0.000 | 0.040 | 0.000 | 0.000 |
| 44 | 1389 | CPXAR | 35.360 | 15.047 | 0.498 | 0.212 | 1.000 | 0.400 | 0.560 | 0.040 | 0.000 | 0.120 | 0.440 |
| 45 | 1392 | AAC6- | 12.960 | 6.661 | 0.230 | 0.119 | 1.000 | 0.920 | 0.080 | 0.000 | 0.000 | 0.000 | 0.000 |
| | | PRIME | | | | | | | | | | | |
| 46 | 1422 | VGBB | 68.640 | 7.416 | 0.766 | 0.084 | 1.000 | 0.960 | 0.040 | 0.000 | 0.000 | 0.080 | 0.277 |
| 47 | 1440 | FOSC | 30.600 | 5.635 | 0.543 | 0.099 | 1.000 | 1.000 | 0.000 | 0.000 | 0.000 | 0.000 | 0.000 |
| 48 | 1535 | LNUA | 18.120 | 10.553 | 0.369 | 0.215 | 1.000 | 0.560 | 0.440 | 0.000 | 0.000 | 0.080 | 0.277 |
| 49 | 1569 | PARE | 151.000 | 41.661 | 0.798 | 0.221 | 0.240 | 0.040 | 0.120 | 0.080 | 0.760 | 0.680 | 2.096 |
| 50 | 1695 | NDM | 50.625 | 16.248 | 0.615 | 0.198 | 0.960 | 0.920 | 0.000 | 0.040 | 0.040 | 0.200 | 1.000 |
| 51 | 1702 | SPG | 70.042 | 13.013 | 0.808 | 0.152 | 0.960 | 0.760 | 0.160 | 0.040 | 0.040 | 0.600 | 1.528 |
| 52 | 1753 | VEB | 72.920 | 15.242 | 0.804 | 0.168 | 1.000 | 0.000 | 0.960 | 0.040 | 0.000 | 0.320 | 0.988 |
| 53 | 1953 | LNUB | 66.480 | 11.435 | 0.820 | 0.142 | 1.000 | 0.720 | 0.280 | 0.000 | 0.000 | 0.200 | 0.408 |
| 54 | 2026 | ERMA | 54.440 | 13.238 | 0.734 | 0.180 | 1.000 | 0.720 | 0.240 | 0.040 | 0.000 | 0.160 | 0.473 |
| 55 | 2357 | SULII | 53.792 | 11.310 | 0.654 | 0.138 | 0.960 | 0.040 | 0.920 | 0.000 | 0.040 | 0.040 | 0.200 |
| 56 | 2517 | TET37 | 19.200 | 6.344 | 0.571 | 0.195 | 1.000 | 1.000 | 0.000 | 0.000 | 0.000 | 0.000 | 0.000 |
| 57 | 2822 | EMRK | 69.833 | 13.127 | 0.656 | 0.125 | 0.960 | 0.880 | 0.040 | 0.040 | 0.040 | 0.160 | 0.624 |
| 58 | 2999 | MPHB | 57.583 | 15.234 | 0.628 | 0.169 | 0.960 | 0.920 | 0.040 | 0.000 | 0.040 | 0.040 | 0.200 |
| 59 | 3024 | VANYM | 52.320 | 8.669 | 0.734 | 0.123 | 1.000 | 0.960 | 0.040 | 0.000 | 0.000 | 0.160 | 0.624 |
| 60 | 3041 | MECC | 172.200 | 24.767 | 0.855 | 0.124 | 1.000 | 0.600 | 0.360 | 0.040 | 0.000 | 0.200 | 1.000 |
| 61 | 3128 | TUFAB | 67.160 | 25.151 | 0.563 | 0.212 | 1.000 | 0.720 | 0.280 | 0.000 | 0.000 | 0.280 | 1.400 |
| 62 | 3176 | AMRB | 308.640 | 16.830 | 0.981 | 0.054 | 1.000 | 0.120 | 0.840 | 0.040 | 0.000 | 0.080 | 0.400 |
| 63 | 3270 | IRI | 133.200 | 16.427 | 0.918 | 0.114 | 1.000 | 0.440 | 0.560 | 0.000 | 0.000 | 0.680 | 0.748 |
| 64 | 3314 | RPOB | 331.667 | 46.523 | 0.931 | 0.131 | 0.480 | 0.000 | 0.480 | 0.000 | 0.520 | 2.440 | 3.429 |
| 65 | 3332 | TET35 | 48.917 | 11.938 | 0.435 | 0.108 | 0.960 | 0.960 | 0.000 | 0.000 | 0.040 | 0.000 | 0.000 |
| 66 | 3370 | CFRA | 87.857 | 8.248 | 0.832 | 0.079 | 0.840 | 0.840 | 0.000 | 0.000 | 0.160 | 0.000 | 0.000 |
| 67 | 3513 | BRP | 17.000 | 8.886 | 0.414 | 0.217 | 0.960 | 0.960 | 0.000 | 0.000 | 0.040 | 0.000 | 0.000 |
| 68 | 3613 | APH3- | 60.240 | 7.833 | 0.741 | 0.097 | 1.000 | 0.920 | 0.080 | 0.000 | 0.000 | 0.080 | 0.277 |
| | | PRIME | | | | | | | | | | | |
| 69 | 3697 | TETM | 175.880 | 19.951 | 0.912 | 0.104 | 1.000 | 0.400 | 0.600 | 0.000 | 0.000 | 1.400 | 1.979 |
| 70 | 3778 | IND | 56.840 | 10.862 | 0.766 | 0.148 | 1.000 | 0.080 | 0.880 | 0.040 | 0.000 | 0.360 | 1.036 |

Simulation settings:
k-mers: ‘constant’, k = 10
Gene coverage: 1
Number of genes: 1
Errors: ‘on’, 0.025
Penalty score: 0.1
Thresholding: multiplier = 2, all factors (1-on)
Entropy screening: ‘rand’

TABLE S9
Resistance genes simulations with 0.05 (10%) error rate

| | | | Avg | StDev | Avg | StDev | | Identification level | | | | | |
|---|---|---|---|---|---|---|---|---|---|---|---|---|---|
| | Data- | Gene | block for | block for | coverage for | coverage for | | (fraction of trials) | | | | Avg | StDev |
| | base | sub- | identifi- | identifi- | identifi- | identifi- | Accu- | Specific | Sub- | | Incor- | false | false |
| No. | No. | class | cation | cation | cation | cation | racy | gene | class | Class | rect | positives | positives |
| 1 | 4 | VANZA | 30.783 | 14.248 | 0.628 | 0.290 | 0.920 | 0.760 | 0.160 | 0.000 | 0.080 | 0.440 | 1.261 |
| 2 | 52 | VANWG | 60.500 | 19.636 | 0.711 | 0.232 | 0.960 | 0.400 | 0.560 | 0.000 | 0.040 | 0.120 | 0.332 |
| 3 | 62 | CTX | 62.240 | 22.946 | 0.706 | 0.261 | 1.000 | 0.000 | 1.000 | 0.000 | 0.000 | 0.480 | 0.823 |
| 4 | 68 | MEFA | 88.600 | 25.492 | 0.723 | 0.209 | 1.000 | 0.360 | 0.640 | 0.000 | 0.000 | 0.280 | 0.542 |
| 5 | 76 | OXA | 50.880 | 20.767 | 0.612 | 0.250 | 1.000 | 0.000 | 1.000 | 0.000 | 0.000 | 0.360 | 0.700 |
| 6 | 92 | CATB | 30.960 | 18.571 | 0.481 | 0.290 | 1.000 | 0.280 | 0.680 | 0.040 | 0.000 | 0.240 | 0.831 |
| 7 | 162 | EREA | 56.364 | 38.857 | 0.457 | 0.315 | 0.880 | 0.320 | 0.560 | 0.000 | 0.120 | 0.160 | 0.624 |
| 8 | 174 | TEM | 55.958 | 15.267 | 0.663 | 0.183 | 0.960 | 0.040 | 0.920 | 0.000 | 0.040 | 0.200 | 0.645 |
| 9 | 193 | CML | 83.800 | 23.836 | 0.633 | 0.181 | 1.000 | 0.280 | 0.680 | 0.040 | 0.000 | 0.080 | 0.400 |
| 10 | 207 | NIMA | 23.917 | 12.014 | 0.513 | 0.263 | 0.960 | 0.760 | 0.160 | 0.040 | 0.040 | 0.480 | 1.686 |
| 11 | 239 | DHFR | 28.360 | 11.018 | 0.587 | 0.230 | 1.000 | 0.400 | 0.600 | 0.000 | 0.000 | 0.000 | 0.000 |
| 12 | 246 | FOLP | 89.720 | 24.630 | 0.749 | 0.206 | 1.000 | 0.960 | 0.040 | 0.000 | 0.000 | 0.080 | 0.400 |
| 13 | 274 | PARC | 207.278 | 32.207 | 0.930 | 0.146 | 0.720 | 0.000 | 0.560 | 0.160 | 0.280 | 2.240 | 2.368 |
| 14 | 276 | SHV | 85.167 | 7.597 | 0.972 | 0.085 | 0.960 | 0.000 | 0.960 | 0.000 | 0.040 | 2.080 | 2.900 |
| 15 | 284 | DFRA | 41.667 | 12.940 | 0.741 | 0.234 | 0.960 | 0.680 | 0.280 | 0.000 | 0.040 | 0.640 | 1.381 |
| 16 | 295 | SOXS | 26.560 | 7.119 | 0.802 | 0.219 | 1.000 | 0.640 | 0.360 | 0.000 | 0.000 | 1.000 | 2.141 |
| 17 | 321 | TSNR | 62.000 | 16.321 | 0.783 | 0.208 | 0.880 | 0.560 | 0.320 | 0.000 | 0.120 | 0.600 | 0.866 |
| 18 | 413 | OMPD | 63.520 | 16.259 | 0.586 | 0.151 | 1.000 | 1.000 | 0.000 | 0.000 | 0.000 | 0.000 | 0.000 |
| 19 | 486 | LMRA | 27.000 | 12.312 | 0.466 | 0.212 | 1.000 | 0.960 | 0.040 | 0.000 | 0.000 | 0.040 | 0.200 |
| 20 | 505 | CARB | 68.583 | 19.440 | 0.758 | 0.214 | 0.960 | 0.680 | 0.280 | 0.000 | 0.040 | 0.520 | 1.046 |
| 21 | 506 | ANT3- | 51.240 | 13.252 | 0.637 | 0.167 | 1.000 | 0.920 | 0.080 | 0.000 | 0.000 | 0.120 | 0.332 |
| | | DPRIME | | | | | | | | | | | |
| 22 | 555 | QNRS | 45.960 | 9.334 | 0.693 | 0.143 | 1.000 | 0.560 | 0.440 | 0.000 | 0.000 | 0.040 | 0.200 |
| 23 | 558 | VANWI | 55.318 | 20.051 | 0.487 | 0.177 | 0.880 | 0.840 | 0.040 | 0.000 | 0.120 | 0.080 | 0.400 |
| 24 | 578 | TETX | 100.960 | 17.714 | 0.862 | 0.153 | 1.000 | 0.360 | 0.640 | 0.000 | 0.000 | 2.080 | 4.271 |
| 25 | 652 | VGA | 111.520 | 32.218 | 0.705 | 0.204 | 1.000 | 0.880 | 0.120 | 0.000 | 0.000 | 0.920 | 2.344 |
| 26 | 682 | MOX | 111.160 | 10.850 | 0.960 | 0.093 | 1.000 | 0.160 | 0.800 | 0.040 | 0.000 | 1.680 | 1.952 |
| 27 | 694 | ACT | 106.720 | 16.794 | 0.851 | 0.134 | 1.000 | 0.120 | 0.880 | 0.000 | 0.000 | 0.440 | 1.003 |
| 28 | 717 | VANRE | 63.542 | 6.547 | 0.907 | 0.095 | 0.960 | 0.640 | 0.320 | 0.000 | 0.040 | 0.640 | 1.150 |
| 29 | 749 | RMTB | 16.810 | 7.420 | 0.640 | 0.287 | 0.840 | 0.640 | 0.200 | 0.000 | 0.160 | 0.800 | 2.021 |
| 30 | 778 | DHA | 62.400 | 29.537 | 0.542 | 0.259 | 1.000 | 0.000 | 1.000 | 0.000 | 0.000 | 0.320 | 0.748 |
| 31 | 789 | CMY | 63.080 | 27.296 | 0.548 | 0.237 | 1.000 | 0.000 | 1.000 | 0.000 | 0.000 | 0.160 | 0.624 |
| 32 | 797 | FACT | 150.000 | 20.613 | 0.887 | 0.123 | 0.760 | 0.480 | 0.280 | 0.000 | 0.240 | 0.400 | 0.764 |
| 33 | 819 | OKP | 84.833 | 4.833 | 0.975 | 0.056 | 0.960 | 0.000 | 0.960 | 0.000 | 0.040 | 2.440 | 2.293 |
| 34 | 973 | IMP | 48.520 | 21.804 | 0.645 | 0.291 | 1.000 | 0.000 | 1.000 | 0.000 | 0.000 | 0.400 | 1.080 |
| 35 | 1048 | VIM | 45.080 | 15.756 | 0.553 | 0.195 | 1.000 | 0.040 | 0.960 | 0.000 | 0.000 | 0.080 | 0.277 |
| 36 | 1135 | PBP1B | 96.444 | 35.196 | 0.389 | 0.142 | 0.720 | 0.720 | 0.000 | 0.000 | 0.280 | 0.080 | 0.400 |
| 37 | 1146 | MPHE | 71.840 | 9.419 | 0.806 | 0.106 | 1.000 | 0.520 | 0.480 | 0.000 | 0.000 | 0.280 | 0.843 |
| 38 | 1182 | VATE | 45.409 | 10.671 | 0.696 | 0.165 | 0.880 | 0.520 | 0.360 | 0.000 | 0.120 | 0.280 | 0.678 |
| 39 | 1214 | OPRJ | 136.227 | 13.596 | 0.940 | 0.094 | 0.880 | 0.360 | 0.520 | 0.000 | 0.120 | 0.600 | 0.645 |
| 40 | 1254 | FOSB | 38.560 | 5.229 | 0.897 | 0.125 | 1.000 | 0.560 | 0.440 | 0.000 | 0.000 | 1.960 | 3.434 |
| 41 | 1271 | VPH | 56.727 | 25.317 | 0.651 | 0.291 | 0.880 | 0.760 | 0.120 | 0.000 | 0.120 | 0.600 | 1.041 |
| 42 | 1283 | SULI | 71.083 | 12.991 | 0.836 | 0.154 | 0.960 | 0.160 | 0.760 | 0.040 | 0.040 | 0.560 | 1.044 |
| 43 | 1297 | TET40 | 41.043 | 21.582 | 0.332 | 0.174 | 0.920 | 0.480 | 0.440 | 0.000 | 0.080 | 0.040 | 0.200 |
| 44 | 1389 | CPXAR | 45.542 | 13.825 | 0.643 | 0.199 | 0.960 | 0.240 | 0.720 | 0.000 | 0.040 | 0.440 | 0.712 |
| 45 | 1392 | AAC6- | 14.760 | 7.423 | 0.261 | 0.132 | 1.000 | 0.800 | 0.200 | 0.000 | 0.000 | 0.000 | 0.000 |
| | | PRIME | | | | | | | | | | | |
| 46 | 1422 | VGBB | 78.600 | 11.281 | 0.875 | 0.130 | 1.000 | 0.680 | 0.320 | 0.000 | 0.000 | 1.400 | 3.109 |
| 47 | 1440 | FOSC | 28.960 | 12.431 | 0.514 | 0.223 | 1.000 | 0.920 | 0.080 | 0.000 | 0.000 | 0.120 | 0.440 |
| 48 | 1535 | LNUA | 25.760 | 12.367 | 0.525 | 0.252 | 1.000 | 0.400 | 0.600 | 0.000 | 0.000 | 0.160 | 0.473 |
| 49 | 1569 | PARE | 164.167 | 32.093 | 0.868 | 0.171 | 0.480 | 0.200 | 0.240 | 0.040 | 0.520 | 1.200 | 2.517 |
| 50 | 1695 | NDM | 59.292 | 13.687 | 0.721 | 0.168 | 0.960 | 0.840 | 0.080 | 0.040 | 0.040 | 0.240 | 0.663 |
| 51 | 1702 | SPG | 76.458 | 14.741 | 0.885 | 0.172 | 0.960 | 0.440 | 0.480 | 0.040 | 0.040 | 0.920 | 0.997 |
| 52 | 1753 | VEB | 77.680 | 19.491 | 0.855 | 0.214 | 1.000 | 0.040 | 0.960 | 0.000 | 0.000 | 0.360 | 0.757 |
| 53 | 1953 | LNUB | 68.042 | 12.267 | 0.839 | 0.152 | 0.960 | 0.720 | 0.240 | 0.000 | 0.040 | 0.160 | 0.374 |
| 54 | 2026 | ERMA | 54.292 | 18.155 | 0.732 | 0.246 | 0.960 | 0.720 | 0.240 | 0.000 | 0.040 | 0.160 | 0.374 |
| 55 | 2357 | SULII | 65.760 | 13.245 | 0.801 | 0.162 | 1.000 | 0.040 | 0.960 | 0.000 | 0.000 | 0.440 | 0.870 |
| 56 | 2517 | TET37 | 22.542 | 6.554 | 0.673 | 0.201 | 0.960 | 0.880 | 0.080 | 0.000 | 0.040 | 0.560 | 2.123 |
| 57 | 2822 | EMRK | 85.500 | 19.269 | 0.804 | 0.184 | 0.880 | 0.640 | 0.160 | 0.080 | 0.120 | 0.320 | 0.627 |
| 58 | 2999 | MPHB | 68.958 | 15.058 | 0.752 | 0.168 | 0.960 | 0.800 | 0.160 | 0.000 | 0.040 | 0.320 | 1.069 |
| 59 | 3024 | VANYM | 62.000 | 9.239 | 0.872 | 0.132 | 0.920 | 0.600 | 0.320 | 0.000 | 0.080 | 0.400 | 0.645 |
| 60 | 3041 | MECC | 189.160 | 19.796 | 0.939 | 0.098 | 1.000 | 0.280 | 0.680 | 0.040 | 0.000 | 0.040 | 0.200 |
| 61 | 3128 | TUFAB | 71.040 | 19.711 | 0.595 | 0.166 | 1.000 | 0.760 | 0.240 | 0.000 | 0.000 | 0.280 | 0.542 |
| 62 | 3176 | AMRB | 305.440 | 22.387 | 0.971 | 0.072 | 1.000 | 0.160 | 0.800 | 0.040 | 0.000 | 0.000 | 0.000 |
| 63 | 3270 | IRI | 140.957 | 8.450 | 0.972 | 0.059 | 0.920 | 0.240 | 0.680 | 0.000 | 0.080 | 1.440 | 1.660 |
| 64 | 3314 | RPOB | 356.000 | 0.000 | 1.000 | 0.000 | 0.200 | 0.000 | 0.200 | 0.000 | 0.800 | 1.320 | 2.750 |
| 65 | 3332 | TET35 | 62.208 | 17.093 | 0.553 | 0.153 | 0.960 | 0.960 | 0.000 | 0.000 | 0.040 | 0.000 | 0.000 |
| 66 | 3370 | CFRA | 86.542 | 16.519 | 0.817 | 0.157 | 0.960 | 0.840 | 0.120 | 0.000 | 0.040 | 0.200 | 0.645 |
| 67 | 3513 | BRP | 20.364 | 9.796 | 0.494 | 0.238 | 0.880 | 0.880 | 0.000 | 0.000 | 0.120 | 0.080 | 0.400 |
| 68 | 3613 | APH3- | 65.583 | 8.075 | 0.808 | 0.101 | 0.960 | 0.880 | 0.080 | 0.000 | 0.040 | 0.440 | 1.446 |
| | | PRIME | | | | | | | | | | | |
| 69 | 3697 | TETM | 184.880 | 15.584 | 0.959 | 0.082 | 1.000 | 0.120 | 0.880 | 0.000 | 0.000 | 1.760 | 2.087 |
| 70 | 3778 | IND | 60.880 | 11.791 | 0.821 | 0.161 | 1.000 | 0.040 | 0.960 | 0.000 | 0.000 | 0.480 | 0.963 |

Simulation settings:
k-mers: ‘constant’, k = 10
Gene coverage: 1
Number of genes: 1
Errors: ‘on’, 0.05
Penalty score: 0.1
Thresholding: multiplier = 2, all factors (1-on)
Entropy screening: ‘rand’

TABLE S10
Resistance genes simulations with 0.10 (20%) error rate

| | | | Avg | StDev | Avg | StDev | | Identification level | | | | | |
|---|---|---|---|---|---|---|---|---|---|---|---|---|---|
| | Data- | Gene | block for | block for | coverage for | coverage for | | (fraction of trials) | | | | Avg | StDev |
| | base | sub- | identifi- | identifi- | identifi- | identifi- | Accu- | Specific | Sub- | | Incor- | false | false |
| No. | No. | class | cation | cation | cation | cation | racy | gene | class | Class | rect | positives | positives |
| 1 | 4 | VANZA | 33.958 | 16.992 | 0.692 | 0.347 | 0.960 | 0.640 | 0.320 | 0.000 | 0.040 | 3.600 | 7.539 |
| 2 | 52 | VANWG | 76.136 | 11.503 | 0.895 | 0.136 | 0.880 | 0.160 | 0.720 | 0.000 | 0.120 | 4.000 | 7.767 |
| 3 | 62 | CTX | 78.160 | 13.530 | 0.888 | 0.154 | 1.000 | 0.000 | 1.000 | 0.000 | 0.000 | 0.760 | 0.970 |
| 4 | 68 | MEFA | 103.600 | 21.747 | 0.845 | 0.178 | 1.000 | 0.200 | 0.800 | 0.000 | 0.000 | 1.960 | 2.894 |
| 5 | 76 | OXA | 65.208 | 23.052 | 0.785 | 0.279 | 0.960 | 0.000 | 0.960 | 0.000 | 0.040 | 2.360 | 3.988 |
| 6 | 92 | CATB | 41.600 | 20.516 | 0.647 | 0.322 | 1.000 | 0.040 | 0.960 | 0.000 | 0.000 | 0.840 | 1.650 |
| 7 | 162 | EREA | 82.438 | 39.744 | 0.670 | 0.324 | 0.640 | 0.280 | 0.360 | 0.000 | 0.360 | 0.920 | 1.778 |
| 8 | 174 | TEM | 67.080 | 20.866 | 0.797 | 0.250 | 1.000 | 0.040 | 0.960 | 0.000 | 0.000 | 1.840 | 2.285 |
| 9 | 193 | CML | 100.520 | 30.206 | 0.761 | 0.229 | 1.000 | 0.040 | 0.880 | 0.080 | 0.000 | 0.200 | 0.408 |
| 10 | 207 | NIMA | 28.400 | 14.483 | 0.614 | 0.316 | 1.000 | 0.640 | 0.280 | 0.080 | 0.000 | 2.120 | 5.876 |
| 11 | 239 | DHFR | 34.320 | 10.135 | 0.712 | 0.213 | 1.000 | 0.120 | 0.880 | 0.000 | 0.000 | 0.920 | 1.730 |
| 12 | 246 | FOLP | 103.750 | 16.308 | 0.868 | 0.137 | 0.960 | 0.600 | 0.360 | 0.000 | 0.040 | 1.320 | 2.376 |
| 13 | 274 | PARC | 209.100 | 28.026 | 0.938 | 0.126 | 0.800 | 0.000 | 0.400 | 0.400 | 0.200 | 2.040 | 2.169 |
| 14 | 276 | SHV | 82.042 | 19.356 | 0.937 | 0.221 | 0.960 | 0.000 | 0.800 | 0.160 | 0.040 | 4.440 | 3.630 |
| 15 | 284 | DFRA | 53.478 | 6.755 | 0.954 | 0.122 | 0.920 | 0.160 | 0.720 | 0.040 | 0.080 | 4.920 | 4.573 |
| 16 | 295 | SOXS | 29.652 | 6.860 | 0.897 | 0.211 | 0.920 | 0.320 | 0.600 | 0.000 | 0.080 | 4.800 | 6.331 |
| 17 | 321 | TSNR | 73.870 | 9.172 | 0.935 | 0.117 | 0.920 | 0.320 | 0.600 | 0.000 | 0.080 | 2.080 | 2.914 |
| 18 | 413 | OMPD | 77.227 | 18.662 | 0.713 | 0.174 | 0.880 | 0.680 | 0.200 | 0.000 | 0.120 | 1.120 | 2.369 |
| 19 | 486 | LMRA | 37.696 | 10.877 | 0.652 | 0.194 | 0.920 | 0.800 | 0.120 | 0.000 | 0.080 | 0.440 | 1.294 |
| 20 | 505 | CARB | 78.875 | 16.894 | 0.870 | 0.189 | 0.960 | 0.360 | 0.520 | 0.080 | 0.040 | 1.680 | 2.495 |
| 21 | 506 | ANT3- | 65.333 | 13.321 | 0.815 | 0.168 | 0.960 | 0.520 | 0.440 | 0.000 | 0.040 | 0.840 | 1.248 |
| | | DPRIME | | | | | | | | | | | |
| 22 | 555 | QNRS | 56.833 | 10.945 | 0.857 | 0.166 | 0.960 | 0.240 | 0.680 | 0.040 | 0.040 | 1.360 | 2.343 |
| 23 | 558 | VANWI | 73.833 | 24.815 | 0.651 | 0.221 | 0.960 | 0.760 | 0.200 | 0.000 | 0.040 | 0.640 | 1.150 |
| 24 | 578 | TETX | 108.130 | 19.398 | 0.922 | 0.166 | 0.920 | 0.120 | 0.800 | 0.000 | 0.080 | 4.280 | 4.686 |
| 25 | 652 | VGA | 124.520 | 36.860 | 0.788 | 0.234 | 1.000 | 0.640 | 0.360 | 0.000 | 0.000 | 2.240 | 2.697 |
| 26 | 682 | MOX | 112.320 | 7.941 | 0.971 | 0.070 | 1.000 | 0.040 | 0.880 | 0.080 | 0.000 | 2.040 | 2.189 |
| 27 | 694 | ACT | 114.600 | 16.304 | 0.914 | 0.131 | 1.000 | 0.080 | 0.920 | 0.000 | 0.000 | 1.400 | 1.500 |
| 28 | 717 | VANRE | 66.280 | 6.374 | 0.946 | 0.092 | 1.000 | 0.400 | 0.600 | 0.000 | 0.000 | 2.400 | 3.379 |
| 29 | 749 | RMTB | 20.320 | 7.915 | 0.778 | 0.308 | 1.000 | 0.520 | 0.480 | 0.000 | 0.000 | 3.000 | 4.397 |
| 30 | 778 | DHA | 88.160 | 34.827 | 0.767 | 0.304 | 1.000 | 0.000 | 0.920 | 0.080 | 0.000 | 1.760 | 3.072 |
| 31 | 789 | CMY | 73.920 | 30.791 | 0.642 | 0.268 | 1.000 | 0.000 | 0.840 | 0.160 | 0.000 | 0.840 | 1.344 |
| 32 | 797 | FACT | 141.263 | 36.689 | 0.835 | 0.217 | 0.760 | 0.440 | 0.320 | 0.000 | 0.240 | 0.680 | 0.988 |
| 33 | 819 | OKP | 84.458 | 7.052 | 0.970 | 0.082 | 0.960 | 0.000 | 0.840 | 0.120 | 0.040 | 4.360 | 4.009 |
| 34 | 973 | IMP | 43.958 | 23.704 | 0.584 | 0.317 | 0.960 | 0.120 | 0.840 | 0.000 | 0.040 | 0.600 | 1.323 |
| 35 | 1048 | VIM | 60.240 | 15.613 | 0.741 | 0.195 | 1.000 | 0.000 | 1.000 | 0.000 | 0.000 | 0.600 | 1.443 |
| 36 | 1135 | PBP1B | 157.091 | 60.552 | 0.634 | 0.245 | 0.440 | 0.360 | 0.080 | 0.000 | 0.560 | 0.240 | 1.012 |
| 37 | 1146 | MPHE | 82.917 | 9.036 | 0.931 | 0.102 | 0.960 | 0.120 | 0.840 | 0.000 | 0.040 | 4.960 | 8.152 |
| 38 | 1182 | VATE | 52.500 | 15.291 | 0.807 | 0.236 | 0.960 | 0.400 | 0.560 | 0.000 | 0.040 | 1.080 | 1.801 |
| 39 | 1214 | OPRJ | 144.773 | 0.429 | 1.000 | 0.000 | 0.880 | 0.000 | 0.880 | 0.000 | 0.120 | 4.160 | 4.160 |
| 40 | 1254 | FOSB | 40.680 | 5.289 | 0.946 | 0.126 | 1.000 | 0.200 | 0.800 | 0.000 | 0.000 | 3.720 | 5.512 |
| 41 | 1271 | VPH | 63.542 | 28.278 | 0.730 | 0.325 | 0.960 | 0.560 | 0.400 | 0.000 | 0.040 | 1.720 | 2.424 |
| 42 | 1283 | SULI | 80.000 | 7.751 | 0.943 | 0.093 | 1.000 | 0.120 | 0.880 | 0.000 | 0.000 | 1.200 | 1.258 |
| 43 | 1297 | TET40 | 53.875 | 29.858 | 0.436 | 0.243 | 0.960 | 0.440 | 0.520 | 0.000 | 0.040 | 0.440 | 1.044 |
| 44 | 1389 | CPXAR | 58.190 | 13.144 | 0.821 | 0.189 | 0.840 | 0.240 | 0.520 | 0.080 | 0.160 | 0.960 | 1.695 |
| 45 | 1392 | AAC6- | 25.600 | 17.448 | 0.455 | 0.313 | 1.000 | 0.560 | 0.440 | 0.000 | 0.000 | 0.240 | 0.723 |
| | | PRIME | | | | | | | | | | | |
| 46 | 1422 | VGBB | 83.870 | 9.739 | 0.935 | 0.110 | 0.920 | 0.320 | 0.600 | 0.000 | 0.080 | 6.880 | 8.192 |
| 47 | 1440 | FOSC | 39.792 | 13.325 | 0.709 | 0.239 | 0.960 | 0.720 | 0.240 | 0.000 | 0.040 | 1.000 | 1.756 |
| 48 | 1535 | LNUA | 32.160 | 16.790 | 0.655 | 0.343 | 1.000 | 0.240 | 0.760 | 0.000 | 0.000 | 2.800 | 4.573 |
| 49 | 1569 | PARE | 183.750 | 14.849 | 0.972 | 0.079 | 0.320 | 0.040 | 0.240 | 0.040 | 0.680 | 1.400 | 2.432 |
| 50 | 1695 | NDM | 68.261 | 20.100 | 0.832 | 0.246 | 0.920 | 0.400 | 0.440 | 0.080 | 0.080 | 2.280 | 3.565 |
| 51 | 1702 | SPG | 80.720 | 11.059 | 0.935 | 0.129 | 1.000 | 0.320 | 0.680 | 0.000 | 0.000 | 2.600 | 2.858 |
| 52 | 1753 | VEB | 79.720 | 14.458 | 0.877 | 0.161 | 1.000 | 0.000 | 1.000 | 0.000 | 0.000 | 1.520 | 3.466 |
| 53 | 1953 | LNUB | 78.400 | 5.909 | 0.968 | 0.073 | 1.000 | 0.200 | 0.760 | 0.040 | 0.000 | 3.440 | 5.394 |
| 54 | 2026 | ERMA | 63.200 | 16.345 | 0.853 | 0.222 | 1.000 | 0.440 | 0.520 | 0.040 | 0.000 | 0.960 | 1.513 |
| 55 | 2357 | SULII | 70.958 | 12.757 | 0.865 | 0.156 | 0.960 | 0.000 | 0.920 | 0.040 | 0.040 | 1.720 | 2.558 |
| 56 | 2517 | TET37 | 27.609 | 6.073 | 0.824 | 0.190 | 0.920 | 0.520 | 0.400 | 0.000 | 0.080 | 4.880 | 10.902 |
| 57 | 2822 | EMRK | 86.348 | 18.458 | 0.813 | 0.174 | 0.920 | 0.560 | 0.280 | 0.080 | 0.080 | 0.800 | 1.581 |
| 58 | 2999 | MPHB | 74.609 | 13.550 | 0.813 | 0.152 | 0.920 | 0.640 | 0.280 | 0.000 | 0.080 | 0.760 | 1.234 |
| 59 | 3024 | VANYM | 64.391 | 9.380 | 0.906 | 0.134 | 0.920 | 0.360 | 0.560 | 0.000 | 0.080 | 6.480 | 8.510 |
| 60 | 3041 | MECC | 193.640 | 16.520 | 0.961 | 0.082 | 1.000 | 0.240 | 0.760 | 0.000 | 0.000 | 0.240 | 0.523 |
| 61 | 3128 | TUFAB | 90.292 | 23.447 | 0.758 | 0.198 | 0.960 | 0.320 | 0.640 | 0.000 | 0.040 | 0.800 | 1.258 |
| 62 | 3176 | AMRB | 294.640 | 70.201 | 0.937 | 0.223 | 1.000 | 0.000 | 0.920 | 0.080 | 0.000 | 0.000 | 0.000 |
| 63 | 3270 | IRI | 142.682 | 7.852 | 0.984 | 0.054 | 0.880 | 0.080 | 0.800 | 0.000 | 0.120 | 4.200 | 3.640 |
| 64 | 3314 | RPOB | 293.000 | 55.648 | 0.823 | 0.157 | 0.160 | 0.000 | 0.160 | 0.000 | 0.840 | 0.320 | 1.600 |
| 65 | 3332 | TET35 | 74.600 | 29.305 | 0.665 | 0.262 | 0.800 | 0.560 | 0.240 | 0.000 | 0.200 | 0.480 | 0.963 |
| 66 | 3370 | CFRA | 95.227 | 16.115 | 0.900 | 0.154 | 0.880 | 0.360 | 0.520 | 0.000 | 0.120 | 4.640 | 6.800 |
| 67 | 3513 | BRP | 29.286 | 9.023 | 0.718 | 0.222 | 0.840 | 0.640 | 0.200 | 0.000 | 0.160 | 2.080 | 7.405 |
| 68 | 3613 | APH3- | 72.792 | 10.266 | 0.898 | 0.128 | 0.960 | 0.520 | 0.440 | 0.000 | 0.040 | 2.720 | 4.440 |
| | | PRIME | | | | | | | | | | | |
| 69 | 3697 | TETM | 183.920 | 18.907 | 0.954 | 0.099 | 1.000 | 0.120 | 0.840 | 0.040 | 0.000 | 3.400 | 2.533 |
| 70 | 3778 | IND | 64.160 | 16.178 | 0.866 | 0.219 | 1.000 | 0.000 | 1.000 | 0.000 | 0.000 | 2.760 | 3.407 |

Simulation settings:
k-mers: ‘constant’, k = 10
Gene coverage: 1
Number of genes: 1
Errors: ‘on’, 0.10
Penalty score: 0.1
Thresholding: multiplier = 2, all factors (1-on)
Entropy screening: ‘rand’

TABLE S11
Simulations with 2 resistance genes (gene combinations and results)

| Combo | Database No. | Database No. | Gene sub-class | Gene sub-class |
|---|---|---|---|---|
| No. | gene 1 | gene 2 | gene 1 | gene 2 |
| 1 | 76 | 505 | OXA | CARB |
| 2 | 92 | 3024 | CATB | VANYM |
| 3 | 1048 | 506 | VIM | ANT3-DPRIME |
| 4 | 1182 | 778 | VATE | DHA |
| 5 | 1702 | 3270 | SPG | IRI |
| 6 | 2357 | 694 | SULII | ACT |
| 7 | 2999 | 68 | MPHB | MEFA |
| 8 | 3041 | 1048 | MECC | VIM |
| 9 | 3128 | 284 | TUFAB | DFRA |
| 10 | 3370 | 3024 | CFRA | VANYM |

| | Avg | StDev | Avg | StDev | | Identification level | | | | | |
|---|---|---|---|---|---|---|---|---|---|---|---|
| | block for | block for | coverage for | coverage for | | (fraction of trials) | | | | Avg | StDev |
| Combo | identifi- | identifi- | identifi- | identifi- | | Specific | Sub- | | | false | false |
| No. | cation | cation | cation | cation | Accuracy | gene | class | Class | Incorrect | positives | positives |
| 1 | 91.042 | 47.288 | 0.534 | 0.265 | 0.960 | 0.100 | 0.760 | 0.100 | 0.040 | 1.000 | 2.000 |
| 2 | 64.188 | 31.773 | 0.481 | 0.255 | 0.960 | 0.300 | 0.640 | 0.020 | 0.040 | 0.280 | 1.021 |
| 3 | 81.959 | 39.022 | 0.511 | 0.232 | 0.980 | 0.060 | 0.900 | 0.020 | 0.020 | 0.880 | 1.900 |
| 4 | 96.060 | 38.859 | 0.536 | 0.212 | 1.000 | 0.140 | 0.840 | 0.020 | 0.000 | 1.240 | 1.715 |
| 5 | 191.283 | 32.959 | 0.821 | 0.147 | 0.920 | 0.640 | 0.260 | 0.020 | 0.080 | 1.080 | 0.812 |
| 6 | 120.040 | 41.134 | 0.597 | 0.177 | 1.000 | 0.060 | 0.940 | 0.000 | 0.000 | 0.000 | 0.000 |
| 7 | 128.771 | 43.605 | 0.605 | 0.205 | 0.960 | 0.280 | 0.680 | 0.000 | 0.040 | 0.040 | 0.200 |
| 8 | 228.604 | 59.409 | 0.831 | 0.199 | 0.960 | 0.440 | 0.060 | 0.460 | 0.040 | 0.600 | 0.957 |
| 9 | 85.634 | 26.347 | 0.489 | 0.162 | 0.820 | 0.700 | 0.120 | 0.000 | 0.180 | 0.120 | 0.440 |
| 10 | 131.500 | 46.095 | 0.754 | 0.256 | 0.880 | 0.580 | 0.300 | 0.000 | 0.120 | 0.440 | 1.193 |

Simulation settings:
k-mers: ‘constant’, k = 10
Gene coverage: 1
Number of genes: 2
Errors: ‘off’
Penalty score: 0.1
Thresholding: multiplier = 4, all factors (1-on)
Entropy screening: ‘rand’

TABLE S12
Simulations with 5 resistance genes (gene combinations and results)

| | Database | Database | Database | Database | Database | Gene | Gene | Gene | Gene | Gene |
|---|---|---|---|---|---|---|---|---|---|---|
| Combo | No. gene | No. gene | No. gene | No. gene | No. gene | sub-class | sub-class | sub-class | sub-class | sub-class |
| No. | 1 | 2 | 3 | 4 | 5 | gene 1 | gene 2 | gene 3 | gene 4 | gene 5 |
| 1 | 295 | 1182 | 819 | 555 | 239 | SOXS | VATE | OKP | QNRS | DHFR |
| 2 | 973 | 2026 | 3041 | 1753 | 694 | IMP | ERMA | MECC | VEB | ACT |
| 3 | 1048 | 3778 | 3270 | 789 | 2517 | VIM | IND | IRI | CMY | TET37 |
| 4 | 3370 | 276 | 1422 | 3778 | 1702 | CFRA | SHV | VGBB | IND | SPG |
| 5 | 3778 | 506 | 274 | 694 | 778 | IND | ANT3- | PARC | ACT | DHA |
| | | | | | | | DPRIME | | | |

| | Avg | StDev | Avg | StDev | | Identification level | | | | | |
|---|---|---|---|---|---|---|---|---|---|---|---|
| | block for | block for | coverage for | coverage for | | (fraction of trials) | | | | Avg | StDev |
| Combo | identifi- | identifi- | identifi- | identifi- | Accu- | Specific | Sub- | | Incor- | false | false |
| No. | cation | cation | cation | cation | racy | gene | class | Class | rect | positives | positives |
| 1 | 158.889 | 91.030 | 0.524 | 0.298 | 0.792 | 0.048 | 0.656 | 0.088 | 0.208 | 4.520 | 4.224 |
| 2 | 441.777 | 186.481 | 0.793 | 0.324 | 0.824 | 0.032 | 0.720 | 0.072 | 0.176 | 9.560 | 5.738 |
| 3 | 245.739 | 130.588 | 0.550 | 0.286 | 0.736 | 0.008 | 0.640 | 0.088 | 0.264 | 3.800 | 3.862 |
| 4 | 281.263 | 138.899 | 0.644 | 0.312 | 0.912 | 0.104 | 0.640 | 0.168 | 0.088 | 11.640 | 7.059 |
| 5 | 330.667 | 168.319 | 0.549 | 0.266 | 0.672 | 0.000 | 0.640 | 0.032 | 0.328 | 4.760 | 5.372 |

Simulation settings:
k-mers: ‘constant’, k = 10
Gene coverage: 1
Number of genes: 5
Errors: ‘off’
Penalty score: 0.1
Thresholding: multiplier = 25, all factors (1-on)
Entropy screening: ‘rand’

TABLE S13
10 randomly-selected cancer genes

| | Gene | | |
|---|---|---|---|
| | database | Sub- | |
| No. | No. | class | Full gene name (from COSMIC database) |
| 1 | 1049 | CMPK1 | CMPK1 ENST00C000371873 1:47333946-47376745(+) |
| 2 | 2851 | C1orf115 | C1orf115 ENST00000294889 1:220690403-220696731(+) |
| 3 | 5025 | MTMR14 | MTMR14 ENST00000296003 3:9649584-9701973(+) |
| 4 | 7924 | CARTPT | CARTPT ENST00000296777 5:71719294-71720615(+) |
| 5 | 9305 | C6orf25 | C6orf25_ENST00000375806 ENST00000375806 6:31723384-31725074(+) |
| 6 | 15404 | FRG2B | FRG23B ENST00000425520 10:133625099-133626742(−) |
| 7 | 19240 | R8M23 | RBM23 ENST00000359890 14:22901730-22911393(−) |
| 3 | 21814 | PDXDC2 | PDXDC2 ENST00000331116 16:69996455-70065776(−) |
| 9 | 24929 | SLC7A10 | SLC7A10 ENST0000025318819:33208891-33225703(−) |
| 10 | 27882 | CSF2RA | CSF2RA ENST00000381S529 23:1282704-1309479(+) |

TABLE S14
Cancer genes simulations

| | | | Avg | StDev | Avg | StDev | | Identification level | | | | | |
|---|---|---|---|---|---|---|---|---|---|---|---|---|---|
| | Data- | Gene | block for | block for | coverage for | coverage for | | (fraction of trials) | | | | Avg | StDev |
| | base | sub- | identifi- | identifi- | identifi- | identifi- | Accu- | Specific | Sub- | | Incor- | false | false |
| No. | No. | class | cation | cation | cation | cation | racy | gene | class | Class | rect | positives | positives |
| 1 | 1049 | CMPK1 | 11.700 | 3.889 | 0.169 | 0.057 | 1.000 | 1.000 | 0.000 | 0.000 | 0.000 | 0.000 | 0.000 |
| 2 | 2851 | C1orf115 | 12.100 | 6.297 | 0.268 | 0.146 | 1.000 | 1.000 | 0.000 | 0.000 | 0.000 | 0.000 | 0.000 |
| 3 | 5025 | MTMR14 | 58.200 | 21.872 | 0.295 | 0.112 | 1.000 | 1.000 | 0.000 | 0.000 | 0.000 | 0.000 | 0.000 |
| 4 | 7924 | CARTPT | 19.600 | 5.758 | 0.536 | 0.160 | 1.000 | 1.000 | 0.000 | 0.000 | 0.000 | 0.000 | 0.000 |
| 5 | 9305 | C6orf25 | 34.200 | 10.326 | 0.467 | 0.142 | 1.000 | 0.900 | 0.100 | 0.000 | 0.000 | 0.000 | 0.000 |
| 6 | 15404 | FRG2B | 25.800 | 8.217 | 0.306 | 0.097 | 1.000 | 1.000 | 0.000 | 0.000 | 0.000 | 0.000 | 0.000 |
| 7 | 19240 | RBM23 | 50.900 | 13.110 | 0.379 | 0.097 | 1.000 | 1.000 | 0.000 | 0.000 | 0.000 | 0.000 | 0.000 |
| 8 | 21814 | PDXDC2 | 39.000 | 10.770 | 0.274 | 0.075 | 1.000 | 1.000 | 0.000 | 0.000 | 0.000 | 0.000 | 0.000 |
| 9 | 24929 | SLC7A10 | 51.300 | 22.081 | 0.324 | 0.139 | 1.000 | 1.000 | 0.000 | 0.000 | 0.000 | 0.000 | 0.000 |
| 10 | 27882 | CSF2RA | 45.900 | 17.451 | 0.377 | 0.143 | 1.000 | 0.400 | 0.600 | 0.000 | 0.000 | 0.000 | 0.000 |

Simulation settings:
k-mers: ‘constant’, k = 10
Gene coverage: 1
Number of genes: 1
Errors: ‘off’
Penalty score: 0.1
Thresholding: multiplier = 1, all factors (1-on)
Entropy screening: ‘rand’

TABLE S15
10 randomly-selected genetic disease genes

| | Gene | | |
|---|---|---|---|
| | databse | Sub- | |
| No. | No. | class | Full gene name (from custom compiled database) |
| 1 | 28 | TBR1 | NG_046904.1 Homo sapiens T-box, brain 1 (TBR1), RefSeqGene on chromosome 2 |
| 2 | 109 | SHH | NG_007504.2 Homo sapiens sonic hedgehog (SHH), RefSeqGene on chromosome 7 |
| 3 | 110 | SIX3 | NG_016222.1 Homo sapiens SIX homeobox 3 (SIX3), RefSeqGene on chromosome 2 |
| 4 | 112 | ZIC2 | NG_007085.3 Homo sapiens Zic family member 2 (ZIC2), RefSeqGene on chromosome 13 |
| 5 | 121 | KRAS | NG_007524.1 Homo sapiens KRAS proto-oncogene, GTPase (KRAS), RefSeqGene on chromosome 12 |
| 6 | 143 | ALAD | NG_008716.1 Homo sapiens aminolevulinate dehydratase (ALAD), RefSeqGene on chromosome 9 |
| 7 | 163 | IGF2 | NG_008849.1 Homo sapiens insulin like growth factor 2 (IGF2), RefSeqGene on chromosome 11 |
| 8 | 202 | PDE6G | NG_009834.1 Homo sapiens phosphodiesterase 6G (PDE6G), RefSeqGene on chromosome 17 |
| 9 | 214 | ROM1 | NG_009845.1 Homo sapiens retinal outer segment membrane protein 1 (ROM1), RefSeqGene on chromosome 11 |
| 10 | 242 | UBA1 | NG_009161.1 Homo sapiens ubiquitin like modifier activating enzyme 1 (UBA1), RefSeqGene on chromosome X |

TABLE S16
Genetic disease genes simulations

| | | | Avg | StDev | Avg | StDev | | Identification level | | | | | |
|---|---|---|---|---|---|---|---|---|---|---|---|---|---|
| | Data- | Gene | block for | block for | coverage for | coverage for | | (fraction of trials) | | | | Avg | StDev |
| | base | sub- | identifi- | identifi- | identifi- | identifi- | | Specific | Sub- | | | false | false |
| No. | No. | class | cation | cation | cation | cation | Accuracy | gene | class | Class | Incorrect | positives | positives |
| 1 | 28 | TBR1 | 70.900 | 43.322 | 0.044 | 0.027 | 1.000 | 1.000 | 0.000 | 0.000 | 0.000 | 0.000 | 0.000 |
| 2 | 109 | SHH | 216.200 | 151.258 | 0.112 | 0.078 | 1.000 | 1.000 | 0.000 | 0.000 | 0.000 | 0.000 | 0.000 |
| 3 | 110 | SIX3 | 119.700 | 38.251 | 0.107 | 0.034 | 1.000 | 1.000 | 0.000 | 0.000 | 0.000 | 0.000 | 0.000 |
| 4 | 112 | ZIC2 | 28.900 | 25.562 | 0.024 | 0.021 | 1.000 | 1.000 | 0.000 | 0.000 | 0.000 | 0.000 | 0.000 |
| 5 | 121 | KRAS | 126.200 | 49.497 | 0.024 | 0.009 | 1.000 | 1.000 | 0.000 | 0.000 | 0.000 | 0.000 | 0.000 |
| 6 | 143 | ALAD | 327.200 | 159.264 | 0.148 | 0.072 | 1.000 | 1.000 | 0.000 | 0.000 | 0.000 | 0.000 | 0.000 |
| 7 | 163 | IGF2 | 186.900 | 161.765 | 0.068 | 0.059 | 1.000 | 1.000 | 0.000 | 0.000 | 0.000 | 0.000 | 0.000 |
| 8 | 202 | PDE6G | 140.500 | 104.140 | 0.107 | 0.079 | 1.000 | 1.000 | 0.000 | 0.000 | 0.000 | 0.000 | 0.000 |
| 9 | 214 | ROM1 | 185.200 | 156.556 | 0.197 | 0.167 | 1.000 | 1.000 | 0.000 | 0.000 | 0.000 | 0.000 | 0.000 |
| 10 | 242 | UBA1 | 1522.300 | 768.633 | 0.486 | 0.245 | 1.000 | 1.000 | 0.000 | 0.000 | 0.000 | 0.000 | 0.000 |

Simulation settings:
k-mers: ‘constant’, k = 10
Gene coverage: 1
Number of genes: 1
Errors: ‘off’
Penalty score: 0.1
Thresholding: multiplier = 3-5, all factors (1-on)
Entropy screening: ‘rand’

### REFERENCES

Each of the below references is hereby incorporated by reference:
1. D. Pushkarev, N. F. Neff, S. R. Quake, Nat. Biotechnol. 2009, 27, 847.
2. H. C. Fan, S. R. Quake, PLoS One 2010, 5, DOI 10.1371/journal.pone.0010439.
3. S. Sharma, T. K. Kelly, P. A. Jones, Carcinogenesis 2009, 31, 27.
4. M. Esteller, Hum. Mol. Genet. 2007, 16, DOI 10.1093/hmg/ddm018.
5. P. W. Laird, Nat. Rev. Genet. 2010, 11, 191.
6. Y. Cheng, N. Xie, P. Jin, T. Wang, Cell Biochem. Funct. 2015, 33, 161.
7. L. Tarayrah, X. Chen, Cell {&} Biosci. 2013, 3, 2.
8. G. P. Pfeifer, W. Xiong, M. A. Hahn, S. G. Jin, Cell Tissue Res. 2014, 356, 631.
9. K. D. Rasmussen, K. Helin, Genes Dev. 2016, 30, 733.
10. G. Ficz, J. G. Gribben, Genomics 2014, 104, 352.
11. X. Deng, R. Su, H. Weng, H. Huang, Z. Li, J. Chen, Cell Res. 2018, 1.
12. K. M. Boycott, M. R. Vanstone, D. E. Bulman, A. E. MacKenzie, Nat. Rev. Genet. 2013, 14, 681.
13. K. M. Boycott, et al., Am. J. Hum. Genet. 2017, 100, 695.
14. F. Sanger, S. Nicklen, A. R. Coulson, Proc. Natl. Acad. Sci. 1977, 74, 5463.
15. M. L. Metzker, Nat. Rev. Genet. 2010, 11, 31.
16. C. W. Fuller, L. R. Middendorf, S. A. Benner, G. M. Church, T. Harris, X. Huang, S. B. Jovanovich, J. R. Nelson, J. A. Schloss, D. C. Schwartz, D. V Vezenov, Nat. Biotechnol. 2009, 27, 1013.
17. D. Branton, D. W. Deamer, A. Marziali, H. Bayley, S. A. Benner, T. Butler, M. Di Ventra, S. Garaj, A. Hibbs, X. Huang, Nat. Biotechnol. 2008, 26, 1146.
18. D. M. Sagar, L. E. Korshoj, K. B. Hanson, P. P. Chowdhury, P. B. Otoupal, A. Chatterjee, P. Nagpal, Small n.d., 1703165.
19. L.-J. Xu, Z.-C. Lei, J. Li, C. Zong, C. J. Yang, B. Ren, J. Am. Chem. Soc. 2015, 137, 5149.
20. E. A. Pozzi, M. D. Sonntag, N. Jiang, J. M. Klingsporn, M. C. Hersam, R. P. Van Duyne, ACS Nano 2013, 7, 885.
21. A. Barhoumi, D. Zhang, F. Tam, N. J. Halas, J. Am. { . . . } 2008, 130, 5523.
22. L. Guerrini, 2. Krpetić, D. Van Lierop, R. A. Alvarez-Puebla, D. Graham, Angew. Chemie—Int. Ed. 2015, 54, 1144.
23. J. Morla-Folch, H. N. Xie, P. Gisbert-Quilis, S. G. De Pedro, N. Pazos-Perez, R. A. Alvarez-Puebla, L. Guerrini, Angew. Chemie—Int. Ed. 2015, 54, 13650.
24. E. J. Blackie, E. C. Le Ru, P. G. Etchegoin, J. Am. Chem. Soc. 2009, 131, 14466.
25. S. Najjar, D. Talaga, L. Schué, Y. Coffinier, S. Szunerits, R. Boukherroub, L. Servant, V. Rodriguez, S. Bonhommeau, J. Phys. Chem. C 2014, 118, 1174.
26. R. Treffer, R. Bohme, T. Deckert-Gaudig, K. Lau, S. Tiede, X. Lin, V. Deckert, Biochem. Soc. Trans. 2012, 40, 609.
27. K. Kneipp, H. Kneipp, V. B. Kartha, R. Manoharan, G. Deinum, I. Itzkan, R. R. Dasari, M. S. Feld, Phys. Rev. E—Stat. Physics, Plasmas, Fluids, Relat. Interdiscip. Top. 1998, 57, DOI 10.1103/PhysRevE.57.R6281.
28. M. S. Schmidt, J. Hübner, A. Boisen, Adv. Mater. 2012, 24, DOI 10.1002/adma.201103496.
29. Q.-C. Sun, Y. C. Ding, D. M. Sagar, P. Nagpal, Prog. Surf. Sci. 2017, DOI https://doi.org/10.1016/j.progsurf.2017.09.003.
30. G. Naja, P. Bouvrette, S. Hrapovic, J. H. Luong, Analyst 2007, 132, 679.
31. G. Kanellis, J. F. Morhange, M. Balkanski, Phys. Rev. B 1980, 21, 1543.
32. E. Galopin, J. Barbillat, Y. Coffinier, S. Szunerits, G. Patriarche, R. Boukherroub, ACS

Appl. Mater. Interfaces 2009, 1, 1396.
33. H. Xu, E. J. Bjerneld, M. Käll, L. Börjesson, Phys. Rev. Lett. 1999, 83, 4357.
34. L. E. Korshoj, S. Afsari, S. Khan, A. Chatterjee, P. Nagpal, Small 2017, 13, 1603033.
35. Hamburg, M. A. & Collins, F. S. The Path to Personalized Medicine. N. Engl. J. Med. 363, 301-304 (2010).
36. Ahmed, M. U., Saaem, I., Wu, P. C. & Brown, A. S. Personalized diagnostics and biosensors: A review of the biology and technology needed for personalized medicine.
37. Crit. Rev. Biotechnol. 34, 180-196 (2014). Ventola, L. The Antibiotic Resistance Crisis. Pharm. Ther. 40, 277-283 (2015).
38. Berendonk, T. U. et al. Tackling antibiotic resistance: The environmental framework. Nat. Rev. Microbiol. 13, 310-317 (2015).
39. Diekema, D. J. & Pfaller, M. A. Rapid detection of antibiotic-resistant organism carriage for infection prevention. Clin. Infect. Dis. 56, 1614-1620 (2013).
40. Strauss, C., Endimiani, A. & Perreten, V. A novel universal DNA labeling and amplification system for rapid microarray-based detection of 117 antibiotic resistance genes in Gram-positive bacteria. J. Microbiol. Methods 108, 25-30 (2015).
41. Perreten, V. et al. Microarray-Based Detection of 90 Antibiotic Resistance Genes of Gram-Positive Bacteria. J Clin Microbiol 43, 2291-2302 (2005).
42. Harrison, L. B. & Hanson, N. D. High-resolution melting analysis for rapid detection of sequence type 131 Escherichia coli. Antimicrob. Agents Chemother. 61, 1-8 (2017).
43. Doumith, M. et al. Rapid identification of major Escherichia coli sequence types causing urinary tract and bloodstream infections. J. Clin. Microbiol. 53, 160-166 (2015).
44. Kalsi, S. et al. Rapid and sensitive detection of antibiotic resistance on a programmable digital microfluidic platform. Lab Chip 15, 3065-3075 (2015).
45. Strommenger, B., Kettlitz, C., Werner, G. & Witte, W. Multiplex PCR Assay for Simultaneous Detection of Nine Clinically Relevant Antibiotic Resistance Genes in Staphylococcus aureus. J. Clin. Microbiol. 41, 4089-4094 (2003).
46. Bogaerts, P. et al. Multicentre evaluation of the BYG Carba v2.0 test, a simplified electrochemical assay for the rapid laboratory detection of carbapenemase-producing Enterobacteriaceae. Sci. Rep. 7, 9937 (2017).
47. Kabir, M. H., Meunier, D., Hopkins, K. L., Giske, C. G. & Woodford, N. A two-centre evaluation of RAPIDEC® CARBA NP for carbapenemase detection in Enterobacteriaceae, Pseudomonas aeruginosa and Acinetobacter spp. J. Antimicrob. Chemother. 71, 1213-1216 (2016).
48. Nair, S. et al. WGS for surveillance of antimicrobial resistance: A pilot study to detect the prevalence and mechanism of resistance to azithromycin in a UK population of nontyphoidal Salmonella. J. Antimicrob. Chemother. 71, 3400-3408 (2016).
49. Walker, T. M. et al. Whole-genome sequencing for prediction of Mycobacterium tuberculosis drug susceptibility and resistance: A retrospective cohort study. Lancet Infect. Dis. 15, 1193-1202 (2015).
50. Goodwin, S., McPherson, J. D. & McCombie, W. R. Coming of age: ten years of nextgeneration sequencing technologies. Nat. Rev. Genet. 17, 333-351 (2016).
51. Branton, D. et al. The potential and challenges of nanopore sequencing. Nat. Biotechnol. 26, 1146-1453 (2008).
52. Tothill, I. E. Biosensors for cancer markers diagnosis. Semin. Cell Dev. Biol. 20, 55-62 (2009).
53. Gahl, W. A. et al. The national institutes of health undiagnosed diseases program: Insights into rare diseases. Genet. Med. 14, 51-59 (2012).
54. Ramoni, R. B. et al. The Undiagnosed Diseases Network: Accelerating Discovery about Health and Disease. Am. J. Hum. Genet. 100, 185-192 (2017).
55. Aéimovié, S. S. et al. LSPR chip for parallel, rapid, and sensitive detection of cancer markers in serum. Nano Lett. 14, 2636-2641 (2014).
56. Zheng, G., Patolsky, F., Cui, Y., Wang, W. U. & Lieber, C. M. Multiplexed electrical detection of cancer markers with nanowire sensor arrays. Nat. Biotechnol. 23, 1294-1301 (2005).
57. Stoeva, S. I., Lee, J. S., Smith, J. E., Rosen, S. T. & Mirkin, C. A. Multiplexed detection of protein cancer markers with biobarcoded nanoparticle probes. J. Am. Chem. Soc. 128, 8378-8379 (2006).
58. Sagar, D. M. et al. High-Throughput Block Optical DNA Sequence Identification. Small 14, 1703165 (2018).
59. Perkins, D. N., Pappin, D. J. C., Creasy, D. M. & Cottrell, J. S. Probability-based protein identification by searching sequence databases using mass spectrometry data. Electrophoresis 20, 3551-3567 (1999).
60. Käll, L., Storey, J. D., MacCoss, M. J. & Noble, W. S. Assigning significance to peptides identified by tandem mass spectrometry using decoy databases. J. Proteome Res. 7, 29-34 (2008).
61. Nesvizhskii, A. I., Vitek, O. & Aebersold, R. Analysis and validation of proteomic data generated by tandem mass spectrometry. Nat. Methods 4, 787-797 (2007).
62. Reinert, K., Langmead, B., Weese, D. & Evers, D. J. Alignment of Next-Generation Sequencing Reads. Annu. Rev. Genomics Hum. Genet. 16, 133-151 (2015).
63. Li, H. & Homer, N. A survey of sequence alignment algorithms for next-generation sequencing. Brief. Bioinform. 11, 473-483 (2010).
64. Lakin, S. M. et al. MEGARes: An antimicrobial resistance database for high throughput sequencing. Nucleic Acids Res. 45, D574-D580 (2017).
65. Lee, A. S. et al. Methicillin-resistant Staphylococcus aureus appendicitis. Nat. Rev. Dis.

Prim. 4, 18033 (2018).
66. Duin, D. van & Paterson, D. Multidrug Resistant Bacteria in the Community: Trends and Lessons Learned. Infect Dis Clin North Am 30, 377-390 (2016).
67. Forbes, S. A. et al. COSMIC: Exploring the world's knowledge of somatic mutations in human cancer. Nucleic Acids Res. 43, D805-D811 (2015).
68. M. Mathlouthi, A.-M. Seuvre, J. L. Koenig, Carbohydr. Res. 1984, 131, 1.
69. M. Mathlouthi, A.-M. Seuvre, J. L. Koenig, Carbohydr. Res. 1984, 134, 23.
70. M. Mathlouthi, A.-M. Seuvre, J. L. Koenig, Carbohydr. Res. 1986, 146, 15.
71. M. Mathlouthi, A.-M. Seuvre, J. L. Koenig, Carbohydr. Res. 1986, 146, 1.
72. C. Otto, T. van den Tweel, F. de Mul, J. Greve, J. Raman Spectrosc. 1986, 17, 289.
73. B. Giese, D. McNaughton, J. Phys. Chem. B 2002, 106, 1461.
74. J. De Gelder, K. De Gussem, P. Vandenabeele, L. Moens, J. Raman Spectrosc. 2007, 38, 1133.
75. S. Martuseviěius, G. Niaura, Z. Talaikyte, V. Razumas, Vib. Spectrosc. 1996, 10, 271.
76. M. Tsuboi, Y. Ezaki, M. Aida, M. Suzuki, A. Yimit, K. Ushizawa, T. Ueda, Biospectroscopy 1998, 4, 61.
77. S. Jarmelo, P. R. Carey, R. Fausto, Vib. Spectrosc. 2007, 43, 104.
78. G. Zhu, X. Zhu, Q. Fan, X. Wan, Spectrochim. Acta—Part A Mol. Biomol. Spectrosc. 2011, 78, 1187.
79. T. Pazderka, V. Kopecký, Spectrochim. Acta—Part A Mol. Biomol. Spectrosc. 2017, 185, 51.
80. Diagnostic Optical Sequencing. ACS Appl. Mater. Interfaces 2019, 11 (39), 35587-35596.

SEQUENCE LISTING

SEQ ID NO. 1
DNA
Cal_1
Artificial
AAAAAAAAAA
SEQ ID NO. 2
DNA
Cal_2
Artificial
GGGGGGGGGG
SEQ ID NO. 3
DNA
Cal_3
Artificial
CCCCCCCCCC
SEQ ID NO. 4
DNA
Cal_4
Artificial
TTTTTTTTTT
SEQ ID NO. 5
DNA
Cal_5
Artificial
AAAGAAAACA
SEQ ID NO. 6
DNA
Cal_6
Artificial
GGGTGGGAGG
SEQ ID NO. 7
DNA
Cal_7
Artificial
CCTCCCACCC
SEQ ID NO. 8
DNA
Cal_8
Artificial
TGTTTTCTTT
SEQ ID NO. 9
DNA
Cal_9
Artificial
AGAATAGAAT
SEQ ID NO. 10
DNA
Cal_10
Artificial
CGGAGGAGCG
SEQ ID NO. 11
DNA
Cal_11
Artificial
CGCTCCGCCT
SEQ ID NO. 12
Cal_12
Artificial
CTTCTATTAT
SEQ ID NO. 13
DNA
Cal_13
Artificial
AACGCATCCA
SEQ ID NO. 14
DNA
Cal_14
Artificial
GTGCGATTGT
SEQ ID NO. 15
DNA
Gen_1
Artificial
CCCACTTTCT
SEQ ID NO. 16
DNA
Gen_2
Artificial
ACGAGGTTCT
SEQ ID NO. 17
DNA
Gen_3
Artificial
GCGCAGGGAG
SEQ ID NO. 18
DNA
Gen_4
Artificial
GATCAGCGCG
SEQ ID NO. 19
DNA
Gen_5
Artificial
CCCCTCCTCT
SEQ ID NO. 20
DNA
Gen_6
Artificial
GGTGGCGAAC
SEQ ID NO. 21
DNA
Gen_7
Artificial
AAGCGCAACG
SEQ ID NO. 22
DNA
Gen_8
Artificial
CTTCGTCCTC
SEQ ID NO. 23
DNA
Gen_9
Artificial
AGCGGCTCTA
SEQ ID NO. 24
DNA
Artificial
GGTGGGTGGG
SEQ ID NO. 25
DNA
Gen_11
Artificial
GACCGGGAGC
SEQ ID NO. 26
DNA
Gen_12
Artificial
GCCAGGTTGT
SEQ ID NO. 27
DNA
Gen_13
Artificial
GCCAATGTCT
SEQ ID NO. 28
DNA
Gen_14
Artificial
AAGCCCCAGC

1. A method of analyzing k-mer content for broad-spectrum sequence recognition comprising the steps of:
applying a Surface-Enhanced Raman Spectroscopy (SERS) substrate to a surface;
directing a light source with a wavelength toward a portion of the SERS substrate, wherein the portion comprises at least 2 or more components;
allowing the light to interact with the portion of the SERS substrate;
detecting the light reflected by the portion of the SERS substrate;
determining the intensity of the Raman shift of the reflected light;
determining the amount of absorbance;
measuring the intensity of Raman shift at one or more wavenumbers and calculating an area under the curve for each measured wavenumber;
determining the relative content of components in the SERS substrate portion based on the relative intensity of the one or more wavenumbers, thereby identifying the k-mer block content in the portion of the SERS substrate; and
inputting the k-mer block content output to a digital computer system which further includes coded instructions executed by said digital computer system including at least one Block Optical Content Scoring (BOCS) algorithm for determining block optical content scoring of said SERS substrate.

2. The method of claim 1 wherein said BOCS algorithm includes one or more of the following functions executed by said digital computer system:
a log block content function configured to generate log of all k-mer blocks and their content;
a sequence mapping function configured to access and scan one or more sequence databases located on a server or network and generate probabilistic determination of target sequences at low coverages;
a scoring function configured to determined the raw probability that a k-mer block content matches the content of the k-length of a sequence in said one or more sequence databases compared to the calculated number of matches that are statistically expected to occur randomly, or alternatively a penalty score function configured to apply a penalty score in place or a raw probability to a k-mer block content that has no identified matches; and
a probability factor function configured to generate a content score for each target sequence in said one or more sequence databases.

3. The method of claim 2 wherein said probability factor function of said BOCS algorithm executed by said digital computer system is further configured to include one or more of the following probability factor functions executed by said digital computer system an configured to generate a content score for each target sequence in said one or more sequence databases:
a first probability factor function (PF₁) configured to generate the cumulative percent difference from average of a normalized raw probability (PDiff) multiplied by a normalized cumulative raw probability;
a second probability factor function (PF₂) configured to generate the total number of blocks, up to the current block, having at least one match from the content alignment;
a third probability factor function (PF₃) configured to generate the product of all normalized raw probabilities taken as the log base 2 sum, which may further generate a negative values, which may be flipped by subtracting from the most negative value;
a fourth probability factor function (PF₄) configured to generate the exponential of the sequence coverage (g_{cov}), indicating the fractional number of individual bases within the target sequence that have been matched during content alignment;
a fifth probability factor function (PF₅) configured to generate the cumulative slope (SPF5) calculated from the percent difference from the average of the PDiff; and
a sixth probability factor function (PF₆) configured to generate the cumulative difference from the average of the PDiff.

4. The method of claim 3 wherein said probability factor function of said BOCS algorithm executed by said digital computer system is further configured to include one or more of the following probability factor functions executed by said digital computer system:
an entropy screening function; and
a thresholding function configured to remove target sequences with lowest probability ranks after each round of block analyses entropy screening.

5. The method of claim 3 wherein the Raman shift measurements are combined with the absorbance measurements to determine the content of the portion of the SERS substrate.

6. The method of claim 5 wherein the Raman shift measurements are combined with the absorbance measurements to determine the content of the portion of the polypeptide that contains modified SERS substrate.

7. The method of claim 6 wherein said SERS substrate is selected from the group consisting of: a polynucleotide, a polypeptide, a modified polynucleotide, a modified polypeptide.

8. The method of claim 7 wherein said modified polynucleotide comprises a modified polynucleotide selected from the group consisting of: a polynucleotide having methylated residues.

9. The method of claim 3 wherein said modified polypeptide comprises a phosphorylated polypeptide.

10. The method of claim 3 wherein said surface comprises is selected form the group consisting of: a plurality of probe tips, and a plurality of charged nanoparticles.

11. The method of claim 10 wherein said wherein said plurality of charged nanoparticles comprises a plurality of positively charged silver (Ag) nanoparticles.

12. The method of claim 3 wherein the Raman shift measurements are combined with the absorbance measurements to determine the content of the portion of the polypeptide.

13. The method of claim 3 wherein said k-mer block content comprises variable length k-mer blocks, or alternatively constant length k-mer blocks.

14. The method of claim 3 wherein the one or more wavenumbers for measuring Raman shift are selected from the wavenumbers in Table 1-3.

15. The method of claim 3 wherein said one or more sequence databases comprises one or more sequence databases selected from the group consisting of:
a gene sequence database;
a protein sequence database;
a biomarker database;
an antibiotic resistance gene database;
the COSMIC cancer database;
NIH Undiagnosed Diseases Network; and
MEGARes database of antimicrobial resistance genes.

16. The method of claim 3 wherein said target sequence comprises a gene or protein sequence.

17. The method of claim 16 wherein said comprises a gene or proteins sequence associate with a disease condition, or antimicrobial resistance.

18. The method of claim 3 wherein said target sequence comprises a biomarker sequence.

19. The method of claim 18 wherein said biomarker comprises a cancer biomarker sequence.

20-30. (canceled)

31. A system for block optical sequence identification comprising:
a surface, comprising a plurality of probes or a plurality of charged nanoparticles configured to be coupled with a Surface-Enhanced Raman Spectroscopy (SERS) substrate;
a laser source;
a light collection device;
at least one spectrophotometer for analyzing the collected light; and
an input and/or output terminal;
a digital computer system;
a storage device;
a communication bus in communication with the laser, collection device, terminal, microprocessor, and storage device.

32. The system of claim 31, wherein the collection device includes at least one notch Raman filter.

33. The system of claim 31, wherein said SERS substrate comprises a substrate selected from the group consisting of: a polynucleotide, a polypeptide, a polynucleotide having modified nucleobases, a polypeptide having modified amino acid bases,

34. The system of claim 33, wherein the a digital computer system further includes coded instructions executed by said digital computer system including at least one BOCS algorithm for determining block optical content storing of said SERS substrate.
